SEQUENCE LISTING (49 sequences)

SEQ ID NO: 1 - protein, 119 aa, Artificial Sequence
  Adeno-Associated Virus 1 - MAAP - AAV1
LEPRSPKPTSKSRTTAGVWCFLATSTSDPSTDSTRGSPSTRRTQRPSSTTRPTTSSSKRVTIRTCGITTPTPSFRSVCKKIRLLGATSGEQSSRPRSGFSNLSVWLRKALRRLLERNVR

SEQ ID NO: 2 - protein, 119 aa, Artificial Sequence
  Adeno-Associated Virus 2 - MAAP - AAV2
LAHHHQSPQSGIRTTAGVLCFLGTSTSDPSTDSTRESRSTRQTPRPSSTTKPTTGSSTAETTRTSSTTTPTRSFRSALKKIRLLGATSDEQSSRRKRGFLNLWAWLRNLLRRLREKRGR

SEQ ID NO: 3 - protein, 119 aa, Artificial Sequence
  Adeno-Associated Virus 3 - MAAP - AAV3
LESLNPKRTNNTRTTVGVLCFRVTNTSDPVTDSTKESRSTRRTRQPSNTTKLTTSSSRPVTTRTSSTTTPTPSFRSVFKKIRLLGATLAEQSSRPKRGSLSLLVWLRKQLKRLLERRGL

SEQ ID NO: 4 - protein, 119 aa, Artificial Sequence
  Adeno-Associated Virus 4 - MAAP - AAV4
LEPLNPRQINNIRTTLGVLCFRVTNTSDPATDSTRGNPSTQRTRQPSSTTRPTTSSSRPVTTPTSSTTTPTRSSSSGFRATHRLGATSAEQSSRPKRGFLNLLVWLSKRVRRLLERRDR

SEQ ID NO: 5 - protein, 118 aa, Artificial Sequence
  Adeno-Associated Virus 5 - MAAP - AAV5
RAHRNQNPISSIKIKPVVLCCLVITISDPETVSIEESLSTGQTRSRESTTSRTTSSLRRETTPTSSTTTRTPSFRRSSPTTHPSGETSERQSFRPRKGFSNLLAWLKRVLRRPLPESG

SEQ ID NO: 6 - protein, 119 aa, Artificial Sequence
  Adeno-Associated Virus 6 - MAAP - AAV6
LEPRNPKPTSKSRTTAGVWCFLATSTSDPSTDSTRGSPSTRRMQRPSSTTRPTTSSSKRVTIRTCGITTPTPSFRSVCKKIRLLGATSGEQSSRPRRGFSNLLVWLRKVLRRLLERNVR

SEQ ID NO: 7 - protein, 119 aa, Artificial Sequence
  Adeno-Associated Virus 7 - MAAP - AAV7
LEPRNPKPTSKSRTTAGVWCFLATSTSDPSTDSTRGSPSTRRTQRPSSTTRPTTSSSKRVTIRTCGITTPTPSFRSVCKKIRHLGATSGEQSSRPRSGFSNLSVWLRKALRRLLQRRDR

SEQ ID NO: 8 - protein, 119 aa, Artificial Sequence
  Adeno-Associated Virus 8 - MAAP - AAV8
LEPRSPKPTSKSRTTAGVWCFLATSTSDPSTDSTRGSPSTRRTQRPSSTTRPTTSSCRRVTIRTCGITTPTPSFRSVCKKIRLLGATSGEQSSRPRSGFSNLSVWLRKALRRLLERRDR

SEQ ID NO: 9 - protein, 119 aa, Artificial Sequence
  Adeno-Associated Virus 9 - MAAP - AAV9
LEPLNPRQINNIKTTLEVLCFRVTNTLDPATDSTRGSRSTQQTRRPSSTTRPTTSSSRPETTRTSSTTTPTPSSRSGSKKIRLLGATSGEQSSRPKRGFLNLLVWLRKRLRRLLERRGL

SEQ ID NO: 10 - protein, 119 aa, Artificial Sequence
  Adeno-Associated Virus 10 - MAAP - AAV10
LEPPSPRPTSRSRTTAGVWCFLATSTSDPSTDSTRGSPSTRRTQRPSSTTRPTTSSSKRVTIRTCGITTPTPSFRSVCKKIRLLGATSGEQSSRPRSGFSNLSVWLRKLLRRLLERRDR

SEQ ID NO: 11 - protein, 119 aa, Artificial Sequence
  Adeno-Associated Virus 11 - MAAP - AAV11
LEPRSPRPTSRSRTTAGVWCFLATSTSDPSTDSTRGSPSTRRTQRPSSTTRPTTSSSKRVTIRTCGITTPTPSFRSVCKKIRLLGATSGEQSSRPRRGYSNLWAWLKKVLKRLLERRDR

SEQ ID NO: 12 - protein, 119 aa, Artificial Sequence
  Adeno-Associated Virus 12 - MAAP - AAV12
LELHNPRPTNSIRTTAGVLCFLGTSTSDPSTDSTRESRSTRQTPRPSSTTRPTTSSSSRGTTRISSTTTPTPSSSSAWRPTPLLGATSGEQSSRPKRGFSSLWVWLKRALKRLLERNAH

SEQ ID NO: 13 - protein, 119 aa, Artificial Sequence
  Adeno-Associated Virus 13 - MAAP - AAV13
LEPLNPRQINNIRTTLGVLCFRVTNTSDPATDLTRGNPSTQRTRQPSNTTRPTTSSSRPVTTPTSSTTTPTPSFRSVFKKIRLLGATSDEQSSRPKRGSLSLWVWLRKRLRRLLEKRDL

SEQ ID NO: 14 - protein, 119 aa, Artificial Sequence
  Adeno-Associated Virus rh.8 - MAAP - AAVrh.8
LEPRNPKPTSKSRTTAGVWCFLATSTSDPSTDSTRGSPSTRRTQRPSSTTKPTTSSSKRVTIRTCGIITPTPSFRSVCKKIRLLGATSGEQSSRPRSGFSNLSVWLRKALRRLLERRDR

SEQ ID NO: 15 - protein, 119 aa, Artificial Sequence
  Adeno-Associated Virus rh.10 - MAAP - AAVrh.10
LEPRNPKPTSKSRTTAGVWCFLATSTSDPSTDSTRGSPSTRRTQRPSSTTRPTTSSSKRVTIRTCGITTPTPSFRSVCKKIRLLGATSGEQSSRPRSGFSNLSVWLRKALRRLLERRDR

SEQ ID NO: 16 - DNA, 2211 bp, Artificial Sequence
  Adeno-Associated Virus 1 - Capsid - AAV1
  misc_feature (80)..(82): n is a, c, g, or t
atggctgccgatggttatcttccagattggctcgaggacaacctctctgagggcattcgcgagtggtgggacttgaaacnnngagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctgtaa

SEQ ID NO: 17 - DNA, 2208 bp, Artificial Sequence
  Adeno-Associated Virus 2 - Capsid - AAV2
  misc_feature (80)..(82): n is a, c, g, or t
atggctgccgatggttatcttccagattggctcgaggacactctctctgaaggaataagacagtggtggaagctcaaacnnngcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 18 - DNA, 2211 bp, Artificial Sequence
  Adeno-Associated Virus 3 - Capsid - AAV3
  misc_feature (80)..(82): n is a, c, g, or t
atggctgctgacggttatcttccagattggctcgaggacaacctttctgaaggcattcgtgagtggtgggctctgaaacnnngagtccctcaacccaaagcgaaccaacaacaccaggacaaccgtcggggtcttgtgcttccgggttacaaatacctcggacccggtaacggactcgacaaaggagagccggtcaacgaggcggacgcggcagccctcgaacacgacaaagcttacgaccagcagctcaaggccggtgacaacccgtacctcaagtacaaccacgccgacgccgagtttcaggagcgtcttcaagaagatacgtcttttgggggcaaccttggcagagcagtcttccaggccaaaaagaggatccttgagcctcttggtctggttgaggaagcagctaaaacggctcctggaaagaagggggctgtagatcagtctcctcaggaaccggactcatcatctggtgttggcaaatcgggcaaacagcctgccagaaaaagactaaatttcggtcagactggagactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttagaggggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactggaactgtcaatcatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtga

SEQ ID NO: 19 - DNA, 2205 bp, Artificial Sequence
  Adeno-Associated Virus 4 - Capsid - AAV4
  misc_feature (77)..(79): n is a, c, g, or t
atgactgacggttaccttccagattggctagaggacaacctctctgaaggcgttcgagagtggtgggcgctgcaacnnngagcccctaaacccaaggcaaatcaacaacatcaggacaacgctcggggtcttgtgcttccgggttacaaatacctcggacccggcaacggactcgacaagggggaacccgtcaacgcagcggacgcggcagccctcgagcacgacaaggcctacgaccagcagctcaaggccggtgacaacccctacctcaagtacaaccacgccgacgcggagttccagcagcggcttcagggcgacacatcgtttgggggcaacctcggcagagcagtcttccaggccaaaaagagggttcttgaacctcttggtctggttgagcaagcgggtgagacggctcctggaaagaagagaccgttgattgaatccccccagcagcccgactcctccacgggtatcggcaaaaaaggcaagcagccggctaaaaagaagctcgttttcgaagacgaaactggagcaggcgacggaccccctgagggatcaacttccggagccatgtctgatgacagtgagatgcgtgcagcagctggcggagctgcagtcgagggcggacaaggtgccgatggagtgggtaatgcctcgggtgattggcattgcgattccacctggtctgagggccacgtcacgaccaccagcaccagaacctgggtcttgcccacctacaacaaccacctctacaagcgactcggagagagcctgcagtccaacacctacaacggattctccaccccctggggatactttgacttcaaccgcttccactgccacttctcaccacgtgactggcagcgactcatcaacaacaactggggcatgcgacccaaagccatgcgggtcaaaatcttcaacatccaggtcaaggaggtcacgacgtcgaacggcgagacaacggtggctaataaccttaccagcacggttcagatctttgcggactcgtcgtacgaactgccgtacgtgatggatgcgggtcaagagggcagcctgcctccttttcccaacgacgtctttatggtgccccagtacggctactgtggactggtgaccggcaacacttcgcagcaacagactgacagaaatgccttctactgcctggagtactttccttcgcagatgctgcggactggcaacaactttgaaattacgtacagttttgagaaggtgcctttccactcgatgtacgcgcacagccagagcctggaccggctgatgaaccctctcatcgaccagtacctgtggggactgcaatcgaccaccaccggaaccaccctgaatgccgggactgccaccaccaactttaccaagctgcggcctaccaacttttccaactttaaaaagaactggctgcccgggccttcaatcaagcagcagggcttctcaaagactgccaatcaaaactacaagatccctgccaccgggtcagacagtctcatcaaatacgagacgcacagcactctggacggaagatggagtgccctgacccccggacctccaatggccacggctggacctgcggacagcaagttcagcaacagccagctcatctttgcggggcctaaacagaacggcaacacggccaccgtacccgggactctgatcttcacctctgaggaggagctggcagccaccaacgccaccgatacggacatgtggggcaacctacctggcggtgaccagagcaacagcaacctgccgaccgtggacagactgacagccttgggagccgtgcctggaatggtctggcaaaacagagacatttactaccagggtcccatttgggccaagattcctcataccgatggacactttcacccctcaccgctgattggtgggtttgggctgaaacacccgcctcctcaaatttttatcaagaacaccccggtacctgcgaatcctgcaacgaccttcagctctactccggtaaactccttcattactcagtacagcactggccaggtgtcggtgcagattgactgggagatccagaaggagcggtccaaacgctggaaccccgaggtccagtttacctccaactacggacagcaaaactctctgttgtgggctcccgatgcggctgggaaatacactgagcctagggctatcggtacccgctacctcacccaccacctgtaa

SEQ ID NO: 20 - DNA, 2175 bp, Artificial Sequence
  Adeno-Associated Virus 5 - Capsid - AAV5
  misc_feature (77)..(79): n is a, c, g, or t
atgtcttttgttgatcaccctccagattggttggaagaagttggtgaaggtcttcgcgagtttttgggccttgaagnnngcccaccgaaaccaaaacccaatcagcagcatcaagatcaagcccgtggtcttgtgctgcctggttataactatctcggacccggaaacggtctcgatcgaggagagcctgtcaacagggcagacgaggtcgcgcgagagcacgacatctcgtacaacgagcagcttgaggcgggagacaacccctacctcaagtacaaccacgcggacgccgagtttcaggagaagctcgccgacgacacatccttcgggggaaacctcggaaaggcagtctttcaggccaagaaaagggttctcgaaccttttggcctggttgaagagggtgctaagacggcccctaccggaaagcggatagacgaccactttccaaaaagaaagaaggctcggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctgttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctttaa

SEQ ID NO: 21 - DNA, 2211 bp, Artificial Sequence
  Adeno-Associated Virus 6 - Capsid - AAV6
  misc_feature (80)..(82): n is a, c, g, or t
atggctgccgatggttatcttccagattggctcgaggacaacctctctgagggcattcgcgagtggtgggacttgaaacnnngagccccgaaacccaaagccaaccagcaaaagcaggacgacggccggggtctggtgcttcctggctacaagtacctcggacccttcaacggactcgacaagggggagcccgtcaacgcggcggatgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggttctcgaaccttttggtctggttgaggaaggtgctaagacggctcctggaaagaaacgtccggtagagcagtcgccacaagagccagactcctcctcgggcattggcaagacaggccagcagcccgctaaaaagagactcaattttggtcagactggcgactcagagtcagtccccgacccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgaacatgggccttgcccacctataacaaccacctctacaagcaaatctccagtgcttcaacgggggccagcaacgacaaccactacttcggctacagcaccccctgggggtattttgatttcaacagattccactgccatttctcaccacgtgactggcagcgactcatcaacaacaattggggattccggcccaagagactcaacttcaagctcttcaacatccaagtcaaggaggtcacgacgaatgatggcgtcacgaccatcgctaataaccttaccagcacggttcaagtcttctcggactcggagtaccagttgccgtacgtcctcggctctgcgcaccagggctgcctccctccgttcccggcggacgtgttcatgattccgcagtacggctacctaacgctcaacaatggcagccaggcagtgggacggtcatccttttactgcctggaatatttcccatcgcagatgctgagaacgggcaataactttaccttcagctacaccttcgaggacgtgcctttccacagcagctacgcgcacagccagagcctggaccggctgatgaatcctctcatcgaccagtacctgtattacctgaacagaactcagaatcagtccggaagtgcccaaaacaaggacttgctgtttagccgggggtctccagctggcatgtctgttcagcccaaaaactggctacctggaccctgttaccggcagcagcgcgtttctaaaacaaaaacagacaacaacaacagcaactttacctggactggtgcttcaaaatataaccttaatgggcgtgaatctataatcaaccctggcactgctatggcctcacacaaagacgacaaagacaagttctttcccatgagcggtgtcatgatttttggaaaggagagcgccggagcttcaaacactgcattggacaatgtcatgatcacagacgaagaggaaatcaaagccactaaccccgtggccaccgaaagatttgggactgtggcagtcaatctccagagcagcagcacagaccctgcgaccggagatgtgcatgttatgggagccttacctggaatggtgtggcaagacagagacgtatacctgcagggtcctatttgggccaaaattcctcacacggatggacactttcacccgtctcctctcatgggcggctttggacttaagcacccgcctcctcagatcctcatcaaaaacacgcctgttcctgcgaatcctccggcagagttttcggctacaaagtttgcttcattcatcacccagtattccacaggacaagtgagcgtggagattgaatgggagctgcagaaagaaaacagcaaacgctggaatcccgaagtgcagtatacatctaactatgcaaaatctgccaacgttgatttcactgtggacaacaatggactttatactgagcctcgccccattggcacccgttacctcacccgtcccctgtaa

SEQ ID NO: 22 - DNA, 2214 bp, Artificial Sequence
  Adeno-Associated Virus 7 - Capsid - AAV7
  misc_feature (80)..(82): n is a, c, g, or t
atggctgccgatggttatcttccagattggctcgaggacaacctctctgagggcattcgcgagtggtgggacctgaaacnnngagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 23 - DNA, 2217 bp, Artificial Sequence
  Adeno-Associated Virus 8 - Capsid - AAV8
  misc_feature (80)..(82): n is a, c, g, or t
atggctgccgatggttatcttccagattggctcgaggacaacctctctgagggcattcgcgagtggtgggcgctgaaacnnngagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 24 - DNA, 2211 bp, Artificial Sequence
  Adeno-Associated Virus 9 - Capsid - AAV9
  misc_feature (80)..(82): n is a, c, g, or t
atggctgccgatggttatcttccagattggctcgaggacaaccttagtgaaggaattcgcgagtggtgggctttgaaacnnngagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 25 - DNA, 2217 bp, Artificial Sequence
  Adeno-Associated Virus 10 - Capsid - AAV10
  misc_feature (80)..(82): n is a, c, g, or t
atggctgctgacggttatcttccagattggctcgaggacaacctctctgagggcattcgcgagtggtgggacctgaaacnnngagcccccaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgttcccccgactcctccacgggcatcggcaagaaaggccagcagcccgctaaaaagagactgaactttgggcagactggcgagtcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaacgggacatcgggaggaagcaccaacgacaacacctacttcggctacagcaccccctgggggtattttgacttcaacagattccactgccacttctcaccacgtgactggcagcgactcatcaacaacaactggggattccggccaaaaagactcagcttcaagctcttcaacatccaggtcaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaatggaagtcaagccgtaggccgttcctccttctactgcctggaatattttccatctcaaatgctgcgaactggaaacaattttgaattcagctacaccttcgaggacgtgcctttccacagcagctacgcacacagccagagcttggaccgactgatgaatcctctcattgaccagtacctgtactacttatccagaactcagtccacaggaggaactcaaggtacccagcaattgttattttctcaagctgggcctgcaaacatgtcggctcaggccaagaactggctgcctggaccttgctaccggcagcagcgagtctccacgacactgtcgcaaaacaacaacagcaactttgcttggactggtgccaccaaatatcacctgaacggaagagactctctggtgaatcccggtgtcgccatggcaacccacaaggacgacgaggaacgcttcttcccgtcgagcggagtcctgatgtttggaaaacagggtgctggaagagacaatgtggactacagcagcgttatgctaacaagcgaagaagaaattaaaaccactaaccctgtagccacagaacaatacggcgtggtggctgacaacttgcagcaagccaatacagggcctattgtgggaaatgtcaacagccaaggagccttacctggcatggtctggcagaaccgagacgtgtacctgcagggtcccatctgggccaagattcctcacacggacggcaactttcacccgtctcctctgatgggcggctttggacttaaacacccgcctccacagatcctgatcaagaacacgccggtacctgcggatcctccaacaacgttcagccaggcgaaattggcttccttcatcacgcagtacagcaccggacaggtcagcgtggaaatcgagtgggagctgcagaaggagaacagcaaacgctggaacccagagattcagtacacttcaaactactacaaatctacaaatgtggactttgctgtcaatacagagggaacttattctgagcctcgccccattggtactcgttatctgacacgtaatctgtaa

SEQ ID NO: 26 - DNA, 2202 bp, Artificial Sequence
  Adeno-Associated Virus 11 - Capsid - AAV11
  misc_feature (80)..(82): n is a, c, g, or t
atggctgctgacggttatcttccagattggctcgaggacaacctctctgagggcattcgcgagtggtgggacctgaaacnnngagccccgaagcccaaggccaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaaaacggctcctggaaagaagagaccgttagagtcaccacaagagcccgactcctcctcgggcatcggcaaaaaaggcaaacaaccagccagaaagaggctcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcgtctcggaacaacatcaagcagcaacacctacaacggattctccaccccctggggatattttgacttcaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagaggggagcctgcctcctttccccaatgacgtgttcatggtgcctcaatatggctactgtggcatcgtgactggcgagaatcagaaccaaacggacagaaacgctttctactgcctggagtattttccttcgcaaatgttgagaactggcaacaactttgaaatggcttacaactttgagaaggtgccgttccactcaatgtatgctcacagccagagcctggacagactgatgaatcccctcctggaccagtacctgtggcacttacagtcgactacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagcttatattccctggaccatctgttaccggaaatacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcatcacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 27 - DNA, 2229 bp, Artificial Sequence
  Adeno-Associated Virus 12 - Capsid - AAV12
  misc_feature (80)..(82): n is a, c, g, or t
atggctgctgacggttatcttccagattggctcgaggacaacctctctgaaggcattcgcgagtggtgggcgctgaaacnnngagctccacaacccaaggccaaccaacagcatcaggacaacggcaggggtcttgtgcttcctgggtacaagtacctcggacccttcaacggactcgacaagggagagccggtcaacgaggcagacgccgcggccctcgagcacgacaaggcctacgacaagcagctcgagcagggggacaacccgtatctcaagtacaaccacgccgacgccgagttccagcagcgcttggcgaccgacacctcttttgggggcaacctcgggcgagcagtcttccaggccaaaaagaggattctcgagcctctgggtctggttgaagagggcgttaaaacggctcctggaaagaaacgcccattagaaaagactccaaatcggccgaccaacccggactctgggaaggccccggccaagaaaaagcaaaaagacggcgaaccagccgactctgctagaaggacactcgactttgaagactctggagcaggagacggaccccctgagggatcatcttccggagaaatgtctcatgatgctgagatgcgtgcggcgccaggcggaaatgctgtcgaggcgggacaaggtgccgatggagtgggtaatgcctccggtgattggcattgcgattccacctggtcagagggccgagtcaccaccaccagcacccgaacctgggtcctacccacgtacaacaaccacctgtacctgcgaatcggaacaacggccaacagcaacacctacaacggattctccaccccctggggatactttgactttaaccgcttccactgccacttttccccacgcgactggcagcgactcatcaacaacaactggggactcaggccgaaatcgatgcgtgttaaaatcttcaacatacaggtcaaggaggtcacgacgtcaaacggcgagactacggtcgctaataaccttaccagcacggttcagatctttgcggattcgacgtatgaactcccatacgtgatggacgccggtcaggaggggagctttcctccgtttcccaacgacgtctttatggttccccaatacggatactgcggagttgtcactggaaaaaaccagaaccagacagacagaaatgccttttactgcctggaatactttccatcccaaatgctaagaactggcaacaattttgaagtcagttaccaatttgaaaaagttcctttccattcaatgtacgcgcacagccagagcctggacagaatgatgaatcctttactggatcagtacctgtggcatctgcaatcgaccactaccggaaattcccttaatcaaggaacagctaccaccacgtacgggaaaattaccactggagactttgcctactacaggaaaaactggttgcctggagcctgcattaaacaacaaaaattttcaaagaatgccaatcaaaactacaagattcccgccagcgggggagacgcccttttaaagtatgacacgcataccactctaaatgggcgatggagtaacatggctcctggacctccaatggcaaccgcaggtgccggggactcggattttagcaacagccagctgatctttgccggacccaatccgagcggtaacacgaccacatcttcaaacaatttgttgtttacctcagaagaggagattgccacaacaaacccacgagacacggacatgtttggacagattgcagataataatcaaaatgccaccaccgcccctcacatcgctaacctggacgctatgggaattgttcccggaatggtctggcaaaacagagacatctactaccagggccctatttgggccaaggtccctcacacggacggacactttcacccttcgccgctgatgggaggatttggactgaaacacccgcctccacagattttcatcaaaaacacccccgtacccgccaatcccaatactacctttagcgctgcaaggattaattcttttctgacgcagtacagcaccggacaagttgccgttcagatcgactgggaaattcagaaggagcattccaaacgctggaatcccgaagttcaatttacttcaaactacggcactcaaaattctatgctgtgggctcccgacaatgctggcaactaccacgaactccgggctattgggtcccgtttcctcacccaccacttgtaa

SEQ ID NO: 28 - DNA, 2202 bp, Artificial Sequence
  Adeno-Associated Virus 13 - Capsid - AAV13
  misc_feature (77)..(79): n is a, c, g, or t
atgactgacggttaccttccagattggctagaggacaacctctctgaaggcgttcgagagtggtgggcgctgcaacnnngagcccctaaacccaaggcaaatcaacaacatcaggacaacgctcggggtcttgtgcttccgggttacaaatacctcggacccggcaacggacttgacaagggggaacccgtcaacgcagcggacgcggcagccctcgaacacgacaaggcctacgaccagcagctcaaggccggtgacaacccctacctcaagtacaaccacgccgacgccgagtttcaggagcgtcttcaagaagatacgtcttttgggggcaacctcggacgagcagtcttccaggccaaaaagaggatccttgagcctctgggtctggttgaggaagcggctaagacggctcctggaaaaaagagacctgtagagcaatctccagcagaaccggactcctcttcgggcatcggcaaatcaggccagcagcccgctagaaaaagactgaattttggtcagactggcgacacagagtcagtcccagaccctcaaccactcggacaacctcccgcagccccctctggtgtgggatctactacaatggcttcaggcggtggcgcaccaatggcagacaataacgagggtgccgatggagtgggtaattcctcaggaaattggcattgcgattcccaatggctgggcgacagagtcatcaccaccagcacccgcacctgggccctgcccacctacaacaatcacctctacaagcaaatctccagccaatcaggagccaccaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactccgagtaccagctcccgtacgtcctcggctcggcgcatcagggatgcctcccgccgttcccagcagacgtcttcatggtcccacagtatggatacctcaccctgaacaacgggagtcaggcggtaggacgctcttccttttactgcctggagtactttccttctcagatgctgcgtactggaaacaactttcagtttagctacacttttgaagacgtgcctttccacagcagctacgctcacagccaaagtctggaccgtctcatgaatcctctgatcgaccagtacctgtactatctgaacaggacacaaacagccagtggaactcagcagtctcggctactgtttagccaagctggacccaccagtatgtctcttcaagctaaaaactggctgcctggaccttgctacagacagcagcgtctgtcaaagcaggcaaacgacaacaacaacagcaactttccctggactggtgccaccaaatatcatctgaatggccgggactcattggtgaacccgggccctgctatggccagtcacaaggatgacaaagaaaagtttttccccatgcatggaaccctgatatttggtaaagaaggaacaaatgccaacaacgcggatttggaaaatgtcatgattacagatgaagaagaaatccgcaccaccaatcccgtggctacggagcagtacgggactgtgtcaaataatttgcaaaactcaaacgctggtccaactactggaactgtcaatcaccaaggagcgttacctggtatggtgtggcaggatcgagacgtgtacctgcagggacccatttgggccaagattcctcacaccgatggacactttcatccttctccactgatgggaggttttgggctcaaacacccgcctcctcagatcatgatcaaaaacactcccgttccagccaatcctcccacaaactttagtgcggcaaagtttgcttccttcatcacacagtactccacggggcaggtcagcgtggagatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 29 - DNA, 2211 bp, Artificial Sequence
  Adeno-Associated Virus rh.8 - Capsid - AAVrh.8
  misc_feature (80)..(82): n is a, c, g, or t
atggctgccgatggttatcttccagattggctcgaggacaacctctctgagggcattcgcgagtggtgggacttgaaacnnngagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaa

SEQ ID NO: 30 - DNA, 2217 bp, Artificial Sequence
  Adeno-Associated Virus rh.10 - Capsid - AAVrh.10
  misc_feature (80)..(82): n is a, c, g, or t
atggctgccgatggttatcttccagattggctcgaggacaacctctctgagggcattcgcgagtggtgggacttgaaacnnngagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccagcagcccgcgaaaaagagactcaactttgggcagactggcgactcagagtcagtgcccgaccctcaaccaatcggagaaccccccgcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctccccacctacaacaaccacctctacaagcaaatctccaacgggacttcgggaggaagcaccaacgacaacacctacttcggctacagcaccccctgggggtattttgactttaacagattccactgccacttctcaccacgtgactggcagcgactcatcaacaacaactggggattccggcccaagagactcaacttcaagctcttcaacatccaggtcaaggaggtcacgcagaatgaaggcaccaagaccatcgccaataaccttaccagcacgattcaggtctttacggactcggaataccagctcccgtacgtcctcggctctgcgcaccagggctgcctgcctccgttcccggcggacgtcttcatgattcctcagtacgggtacctgactctgaacaatggcagtcaggccgtgggccgttcctccttctactgcctggagtactttccttctcaaatgctgagaacgggcaacaactttgagttcagctaccagtttgaggacgtgccttttcacagcagctacgcgcacagccaaagcctggaccggctgatgaaccccctcatcgaccagtacctgtactacctgtctcggactcagtccacgggaggtaccgcaggaactcagcagttgctattttctcaggccgggcctaataacatgtcggctcaggccaaaaactggctacccgggccctgctaccggcagcaacgcgtctccacgacactgtcgcaaaataacaacagcaactttgcctggaccggtgccaccaagtatcatctgaatggcagagactctctggtaaatcccggtgtcgctatggcaacccacaaggacgacgaagagcgattttttccgtccagcggagtcttaatgtttgggaaacagggagctggaaaagacaacgtggactatagcagcgttatgctaaccagtgaggaagaaattaaaaccaccaacccagtggccacagaacagtacggcgtggtggccgataacctgcaacagcaaaacgccgctcctattgtaggggccgtcaacagtcaaggagccttacctggcatggtctggcagaaccgggacgtgtacctgcagggtcctatctgggccaagattcctcacacggacggaaactttcatccctcgccgctgatgggaggctttggactgaaacacccgcctcctcagatcctgattaagaatacacctgttcccgcggatcctccaactaccttcagtcaagctaagctggcgtcgttcatcacgcagtacagcaccggacaggtcagcgtggaaattgaatgggagctgcagaaagaaaacagcaaacgctggaacccagagattcaatacacttccaactactacaaatctacaaatgtggactttgctgttaacacagatggcacttattctgagcctcgccccatcggcacccgttacctcacccgtaatctgtaa

SEQ ID NO: 31 - DNA, 25 bp, Artificial Sequence
  AAV2-ITR (fwd)
aacatgctacgcagagagggagtgg

SEQ ID NO: 32 - DNA, 29 bp, Artificial Sequence
  AAV2-ITR2 (rev)
catgagacaaggaacccctagtgatggag

SEQ ID NO: 33 - protein, 1053 aa, Artificial Sequence
  Staphylococcus Aureus - Cas9
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 34 - DNA, 3162 bp, Artificial Sequence
  Staphylococcus Aureus - Cas9/Csn1
atgaaaaggaattatatcttaggattagatatcggaattacatcagtgggttatggaattattgattatgaaactagagatgtcatagatgcgggcgtacgtttatttaaagaggctaatgttgaaaataatgaaggacgacgatcaaaaagaggtgccagaaggcttaagaggcgtcgtagacatagaatacaaagagtaaagaaacttttatttgattacaatttgttgacagatcatagtgagctaagtggaatcaatccttacgaggcgcgcgtaaagggattaagtcaaaaattaagtgaagaggaattttctgcggcattgctacatttagcaaagcgtagaggtgtacataatgttaatgaagtggaagaagatacaggtaatgaattatccactaaagaacaaatttcaagaaatagtaaagcgttagaagagaagtatgttgcagaattacagttggaacgtttgaaaaaagacggtgaagtgagaggttcgattaaccgtttcaaaacatctgactatgtaaaagaagcaaagcagttattaaaagtacaaaaagcatatcatcaacttgatcaatcatttatagacacttatattgatttattggaaacaagaagaacatattatgagggaccaggtgaaggtagcccatttggatggaaagatattaaagaatggtatgaaatgttaatgggacattgtacgtatttcccagaagaattacgtagtgtgaaatatgcctataatgctgatttatataatgcgctgaatgatttgaacaacttggttattacacgagatgagaatgagaagctagagtattatgaaaaattccaaattatcgagaatgtctttaaacaaaagaaaaagccgacgcttaaacaaattgcgaaggaaatcttggtgaatgaagaagacatcaaaggctatcgtgtcacaagtacaggtaaaccagaatttacaaacttgaaagtttatcacgatatcaaagatattacagcaagaaaagaaattatcgagaatgcagagctactcgatcaaatagctaaaatattaactatttaccagtcatcagaagatatacaagaagaattaacaaacctaaattcagaattgacacaagaagagattgaacaaatttcaaacttgaaaggttatacaggaactcataacctttcactaaaggcaataaatttaatattagacgaattgtggcatacgaacgataatcaaatagctattttcaatcgtttgaaacttgtacctaaaaaggtagatttaagccaacaaaaagaaattcctactactttagttgatgattttatactgtctccagtagtgaaacgttcatttatacaatctattaaagttattaacgctattattaaaaaatacggtttgccaaatgatattattattgaacttgcgagagaaaagaattctaaagatgcacaaaaaatgattaatgaaatgcagaagagaaatcgtcaaacgaatgaacgtattgaggaaattataagaacgacaggtaaagaaaatgctaaatatttaattgaaaaaattaagctgcacgatatgcaagaagggaaatgtttatactcgttagaagcaatccctctagaagatttacttaataatccattcaattacgaagtagaccatatcattccacgttctgtttctttcgataactctttcaataataaagtgttggtgaaacaagaagaaaatagtaaaaaaggtaatagaacgccatttcaatatttaagttcttcagattctaaaataagttatgagacattcaaaaagcatattttaaatcttgctaaaggcaaaggtagaatctctaagacgaaaaaagaatatttgttagaagaacgagatatcaatcgcttcagtgtccaaaaagattttattaaccgtaacttagtagatacacgctatgcgacaagagggttaatgaacttattaagatcttattttagagtgaataacttagatgtcaaagtgaaatcgattaatggcggattcacaagtttcttaagaaggaaatggaagttcaaaaaagaaagaaataagggctacaaacaccatgctgaagatgcactgattattgcgaacgctgattttattttcaaagaatggaaaaaactagataaagctaaaaaagtgatggaaaatcaaatgtttgaagaaaagcaagctgaaagtatgcctgaaattgagactgagcaagagtataaagaaatttttataacgcctcatcaaattaaacatattaaggattttaaagattataaatattcacatagagttgataaaaagccgaatagagagttaataaatgatacattatattctacgagaaaagatgacaagggtaatacattaatcgttaataacttaaatggtttatacgataaagataatgataaattgaaaaaattaattaataaatcacctgaaaaattattgatgtatcatcatgatccacaaacatatcaaaaattaaaattgatcatggaacaatatggcgatgagaaaaatccgctttataaatattatgaagaaacaggcaattacttaacaaaatatagtaaaaaagataacggaccagtcatcaaaaaaattaaatattatggtaacaagctaaatgcgcatttagatattacggatgattatccaaatagcagaaataaagtagtaaaactttcattaaaaccatatcgctttgatgtttatttagataatggggtatataaatttgtgacagttaaaaatttagatgttatcaaaaaagaaaactactatgaagttaattcaaagtgttatgaagaagcaaaaaaactgaagaaaattagtaatcaagcagaatttatcgcaagtttttacaataatgacttgattaagattaacggagaattatatagagtcataggtgtaaataatgatctacttaacagaattgaagtaaatatgatagacatcacatatagagaatatttagagaacatgaatgataaaagaccacctagaataattaaaacaatagcaagcaaaacacaatctattaaaaagtattctacagatattctaggcaatctttatgaagtgaagagtaaaaagcatcctcaaatcataaagaaaggatga

SEQ ID NO: 35 - protein, 1368 aa, Artificial Sequence
  Streptococcus Pyogenese Serotype M2 - Cas9 - SP
MDKKYSIGLDIGTNSVGWAVITDDYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRLNSEITKAPLSASMIKRYDEHHQDLTLLKALVRQQLPEKYKEIFFDQSKNGYAGYIDGGASQEEFYKFIKPILEKMDGTEELLAKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDHIVPQSFIKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGLTIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 36 - protein, 97 aa, Artificial Sequence
  AAV8 - MAAP8 - Delta N
LTSTSDPSTDSTRGSPSTRRTQRPSSTTRPTTSSCRRVTIRTCGITTPTPSFRSVCKKIRLLGATSGEQSSRPRSGFSNLSVWLRKALRRLLERRDR

SEQ ID NO: 37 - protein, 69 aa, Artificial Sequence
  AAV8 - MAAP8 - Delta L
LEPRSPKPTSKSRTTAGVWCFLAFRSVCKKIRLLGATSGEQSSRPRSGFSNLSVWLRKALRRLLERRDR

SEQ ID NO: 38 - protein, 47 aa, Artificial Sequence
  AAV8 - MAAP8 - Delta NL
LFRSVCKKIRLLGATSGEQSSRPRSGFSNLSVWLRKALRRLLERRDR

SEQ ID NO: 39 - protein, 73 aa, Artificial Sequence
  AAV8 - MAAP8 - Delta C
LEPRSPKPTSKSRTTAGVWCFLATSTSDPSTDSTRGSPSTRRTQRPSSTTRPTTSSCRRVTIRTCGITTPTPS

SEQ ID NO: 40 - protein, 112 aa, Artificial Sequence
  AAV8 - MAAP8 - Delta 1
LEPRSPKPTSKSRTTATSTSDPSTDSTRGSPSTRRTQRPSSTTRPTTSSCRRVTIRTCGITTPTPSFRSVCKKIRLLGATSGEQSSRPRSGFSNLSVWLRKALRRLLERRDR

SEQ ID NO: 41 - protein, 102 aa, Artificial Sequence
  AAV8 - MAAP8 - Delta 2
LEPRSPKPTSKSRTTAGVWCFLARRTQRPSSTTRPTTSSCRRVTIRTCGITTPTPSFRSVCKKIRLLGATSGEQSSRPRSGFSNLSVWLRKALRRLLERRDR

SEQ ID NO: 42 - protein, 108 aa, Artificial Sequence
  AAV8 - MAAP8 - Delta 3
LEPRSPKPTSKSRTTAGVWCFLATSTSDPSTDSTRGSPSTRRTQRPRRVTIRTCGITTPTPSFRSVCKKIRLLGATSGEQSSRPRSGFSNLSVWLRKALRRLLERRDR

SEQ ID NO: 43 - protein, 103 aa, Artificial Sequence
  AAV8 - MAAP8 - Delta 4
LEPRSPKPTSKSRTTAGVWCFLATSTSDPSTDSTRGSPSTRRTQRPSSTTRPTTSSCRRVVCKKIRLLGATSGEQSSRPRSGFSNLSVWLRKALRRLLERRDR

SEQ ID NO: 44 - protein, 112 aa, Artificial Sequence
  AAV8 - MAAP8 - Delta 5
LEPRSPKPTSKSRTTAGVWCFLATSTSDPSTDSTRGSPSTRRTQRPSSTTRPTTSSCRRVTIRTCGITTPTPSFRSVCKKIRLLGARPRSGFSNLSVWLRKALRRLLERRDR

SEQ ID NO: 45 - protein, 111 aa, Artificial Sequence
  AAV8 - MAAP8 - Delta 6
LEPRSPKPTSKSRTTAGVWCFLATSTSDPSTDSTRGSPSTRRTQRPSSTTRPTTSSCRRVTIRTCGITTPTPSFRSVCKKIRLLGATSGEQSSRPRSGRKALRRLLERRDR

SEQ ID NO: 46 - protein, 106 aa, Artificial Sequence
  AAV8 - MAAP8 - Delta 7
LEPRSPKPTSKSRTTAGVWCFLATSTSDPSTDSTRGSPSTRRTQRPSSTTRPTTSSCRRVTIRTCGITTPTPSFRSVCKKIRLLGATSGEQSSRPRSGFSNLSVWL

SEQ ID NO: 47 - protein, 94 aa, Artificial Sequence
  AAV8 - MAAP8 - Delta 1-2
LEPRSPKPTSKSRTTRRTQRPSSTTRPTTSSCRRVTIRTCGITTPTPSFRSVCKKIRLLGATSGEQSSRPRSGFSNLSVWLRKALRRLLERRDR

SEQ ID NO: 48 - protein, 77 aa, Artificial Sequence
  AAV8 - MAAP8 - Delta 1-3
LEPRSPKPTSKSRTTRRVTIRTCGITTPTPSFRSVCKKIRLLGATSGEQSSRPRSGFSNLSVWLRKALRRLLERRDR

SEQ ID NO: 49 - protein, 58 aa, Artificial Sequence
  AAV8 - MAAP8 - Delta 1-4
LEPRSPKPTSKSRTTVCKKIRLLGATSGEQSSRPRSGFSNLSVWLRKALRRLLERRDR

(19) United States
(12) Patent Application Publication
(10) Pub. No.: US 2024/0294576 A1
(43) Pub. Date: Sep. 5, 2024

(54) COMPOSITIONS AND METHODS FOR FORMATION AND SECRETION OF EXTRACELLULAR VESICLES AND AAV PARTICLES

(71) Applicant: DUKE UNIVERSITY, Durham, NC (US)

(72) Inventors: Aravind Asokan, Durham, NC (US); Zachary Elmore, Durham, NC (US); Lawrence Patrick Havlik, Durham, NC (US); Daniel K. Oh, Durham, NC (US)

(73) Assignee: DUKE UNIVERSITY, Durham, NC (US)

(21) Appl. No.: 17/919,997

(22) PCT Filed: May 5, 2021
(86) PCT No.: PCT/US21/30915

§ 371 Date: Oct. 19, 2022

Related U.S. Application Data
(60) Provisional application No. 63/123,668, filed on Dec. 10, 2020.
(60) Provisional application No. 63/020,051, filed on May 5, 2020.

(51) Int. Cl. C07K 14/005; A61K 38/46; A61K 47/64; C12N 15/86

(52) U.S. Cl. CPC C07K 14/005; A61K 38/465; A61K 47/64; C12N 15/86; C12N 2750/14122; C12N 2750/14143; C12N 2750/14152

(57) ABSTRACT

Disclosed herein are compositions and methods for enhancing secretion of extracellular vesicles and/or AAV particles from cells. Disclosed herein are compositions and methods for altering and/or modifying the formation and/or secretion of extracellular vesicles and/or AAV particles from cells. Disclosed herein are compositions and methods for loading extracellular vesicles and or AAV particles with a cargo. Disclosed herein are isolated nucleic acid molecules encoding a polypeptide for promoting the formation of extracellular vesicles and AAV particles in cell or a polypeptide associated with extracellular vesicles and/or AAV particles secreted from a cell.

### CROSS-REFERENCE TO RELATED APPLICATIONS

This application claims the benefit of U.S. Provisional Application No. 63/020,051 filed 5 May 2020 and the benefit of U.S. Provisional Application No. 63/123,668 filed 10 Dec. 2020, both of which are incorporated by reference herein in their entirety.

### STATEMENT REGARDING FEDERALLY SPONSORED RESEARCH

Parts of this invention were made with government support under Grant Numbers R01HL089221, UG3AR07336, R01GM127709, and R01NS099371 awarded by the National Institutes of Health.

### REFERENCE TO THE SEQUENCE LISTING

The Sequence Listing submitted 5 May 2021 as a text file named “21_2005_WO Sequence Listing”, created on 5 May 2021 and having a size of 100 kilobytes is hereby incorporated by reference pursuant to 37 C.F.R. § 1.52(e)(5).

### BACKGROUND OF THE INVENTION

Recombinant adeno-associated virus (rAAV) vectors are a leading gene delivery platform, and several rAAV-mediated therapies have recently been approved. Despite these advances in the clinic, rAAV vector manufacturing remains a challenge. Adeno-associated viruses (AAV) are non-enveloped, parvoviruses that rely on a helper virus for transitioning from a latent to lytic cycle. (Uloha A I, et al. 2003 Endokrynologia, Diabetologia i Choroby Przemiany Materii Wieku Rozwojowego. 9(2):73-76). Upon co-infection with a helper such as adenovirus, herpesvirus, or papillomavirus, the dependoparvovirus AAV undergoes a transition from latent to lytic life cycle, exploiting the hijacked host cell machinery. (Uloha A I, et al. 2003). A significant nucleolar buildup of AAV particles has been demonstrated following synthesis and replication of the single-stranded DNA (ssDNA) AAV genome, capsid assembly and packaging, all of which occur within nuclear loci. (Wistuba A, et al. 1997 J Virol. 71(2):1341-1352). Notably, unlike other autonomous parvoviruses that undergo a lytic cycle (Uloha A I, et al. 2003), AAV does not induce marked cytopathic effects (CPE). Nevertheless, some recombinant AAV serotypes appear to be secreted into cell culture media prior to lysis, albeit with variable efficiency. (Vandenberghe L H, et al. 2010 Hum Gene Ther. 21(10): 1251-1257; Piras B A, et al. 2016 Mol Ther Methods Clin Dev. 3:16015; Lock M. et al. 2010 Hum Gene Ther. 21(10):1259-1271; Okada T, et al. 2009 Hum Gene Ther. 20(9):1013-1021; Benskey M J, et al. 2016 Hum Gene Ther Methods. 27(1):32-45). To this end, some recombinant AAV serotypes are secreted in a pre-lytic manner as free particles or particles associated with “extracellular vesicles” (EVs), which are released into the supernatant fraction of the cell culture media. (Maguire C A, et al. 2012 Mol Ther. 20(5):960-971; György B, et al. 2018 Wiley Interdiscip Rev Nanomedicine Nanobiotechnology. 10(3):e1488).

Although the cellular egress of virions is thought to be primarily driven by overexpression of Adenoviral or Herpesvirus proteins (Buller R M L, et al. 1981 J Virol. 40(1):241-247: Janik J E, et al. 1981 Proc Natl Acad Sci USA. 78(3):1925-1929; Meier A F, et al. 2020 Viruses. 12(6):662; Smith G A, et al. (2002) Annu Rev Cell Dev Biol. 18:135-161), exactly how AAV exits the cell upon transitioning into this phase of replication was previously unclear. Because AAVs are commonly used for gene therapy, understanding the mechanism driving cellular secretion of AAVs and extracellular vesicles generally is needed.

Accordingly, there is a need to control the ability of a cell to form and secrete extracellular vesicles and/or AAV particles following an AAV infection or following the expression of nucleic acid molecule encoding a MAAP or a fragment thereof.

The data provided herein confirm that the membrane-associated accessory protein (MAAP), which is expressed from a (+1) frameshifted open reading frame (ORF) in the N-terminal region of the AAV capsid (Cap) gene, is an AAV cellular egress factor.

### BRIEF SUMMARY OF THE INVENTION

Disclosed herein is a membrane-associated protein (MAAP) derived from an alternate reading frame in the genome sequence of an Adeno-Associated Virus (AAV), wherein MAAP promotes the formation of extracellular vesicles and/or AAV particles in a mammalian cell, and wherein MAAP comprises the sequence set forth in any one of SEQ ID NO. 01-SEQ ID NO:15.

Disclosed herein is a membrane-associated accessory protein (MAAP) derived from an alternate reading frame in the genome sequence of an Adeno-Associated Virus (AAV), wherein MAAP associates with extracellular vesicles and/or AAV particles secreted from a mammalian cell, and wherein MAAP comprises the sequence set forth in any one of SEQ ID NO:01-SEQ ID NO:15.

Disclosed herein is a membrane-associated accessory protein (MAAP) comprising the sequence set forth in any one of SEQ ID NO:01-SEQ ID NO:15, wherein MAAP comprises an N-terminal domain connected to a C-terminal cationic, amphipathic membrane anchoring domain through a linker domain.

Disclosed herein is a membrane-associated accessory protein (MAAP) derived from an alternate reading frame in the genome sequence of an Adeno-Associated Virus (AAV), wherein MAAP promotes the formation of extracellular vesicles and/or AAV particles in a mammalian cell; and wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15.

Disclosed herein is a membrane-associated accessory protein (MAAP) derived from an alternate reading frame in the genome sequence of an Adeno-Associated Virus (AAV), wherein MAAP associates with extracellular vesicles and/or AAV particles secreted from a mammalian cell; and wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15.

Disclosed herein is a membrane-associated accessory protein (MAAP) comprising the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

Disclosed herein is an AAV capsid gene sequence comprising the sequence set forth in any one of SEQ ID NO:16-SEQ ID NO:30, wherein the sequence encodes a membrane-associated accessory protein (MAAP) when read in an alternate reading frame. Table 2 shows the serotype for each of SEQ ID NO:16-SEQ ID NO:30. Table 4 provides the nucleotide sequence for each of SEQ ID NO:16-SEQ ID NO:30.

Disclosed herein is an isolated nucleic acid molecule comprising a nucleic acid sequence encoding a polypeptide for promoting the formation of extracellular vesicles and/or AAV particles in cell.

Disclosed herein is an isolated nucleic acid molecule comprising a nucleic acid sequence encoding a polypeptide associated with extracellular vesicles and/or AAV particles secreted from a cell.

Disclosed herein is an isolated nucleic acid molecule comprising a nucleic acid sequence encoding a polypeptide for promoting the formation of extracellular vesicles and/or AAV particles in a cell and at least one therapeutic agent.

Disclosed herein is an isolated nucleic acid molecule comprising a nucleic acid sequence encoding a polypeptide associated with extracellular vesicles and/or AAV particles secreted from a cell and at least one therapeutic agent.

Disclosed herein is an isolated nucleic acid molecule comprising a nucleic acid sequence encoding a polypeptide for promoting the formation of extracellular vesicles and/or AAV particles in a cell and an endonuclease.

Disclosed herein is an isolated nucleic acid molecule comprising a nucleic acid sequence encoding a polypeptide associated with extracellular vesicles and/or AAV particles secreted from a cell and an endonuclease.

Disclosed herein is an isolated nucleic acid molecule comprising a nucleic acid sequence encoding a fusion product, wherein the fusion product comprises a polypeptide for promoting the formation of extracellular vesicles and/or AAV particles secreted from a cell and at least one therapeutic agent.

Disclosed herein is an isolated nucleic acid molecule comprising a nucleic acid sequence encoding a fusion product, wherein the fusion product comprises a polypeptide associated with extracellular vesicles and/or AAV particles secreted from a cell and at least one therapeutic agent.

Disclosed herein is an isolated nucleic acid molecule comprising a nucleic acid sequence encoding a fusion product, wherein the fusion product comprises a polypeptide for promoting the formation of extracellular vesicles and/or AAV particles secreted from a cell and an endonuclease.

Disclosed herein is an isolated nucleic acid molecule comprising a nucleic acid sequence encoding a fusion product, wherein the fusion product comprises a polypeptide associated with extracellular vesicles and/or AAV particles secreted from a cell and an endonuclease.

Disclosed herein is a fusion product comprising a polypeptide for promoting the formation of extracellular vesicles and/or AAV particles in a cell and at least one therapeutic agent.

Disclosed herein is a fusion product comprising a polypeptide associated with extracellular vesicles and/or AAV particles secreted from a cell and at least one therapeutic agent.

Disclosed herein is a fusion product comprising a polypeptide for promoting the formation of extracellular vesicles and/or AAV particles in a cell and an endonuclease.

Disclosed herein is a fusion product comprising a polypeptide associated with extracellular vesicles and/or AAV particles secreted from a cell and an endonuclease.

Disclosed herein is a vector comprising an isolated nucleic acid molecule comprising a nucleic acid sequence encoding a polypeptide for promoting the formation of extracellular vesicles and/or AAV particles in cell.

Disclosed herein is a vector comprising an isolated nucleic acid molecule comprising a nucleic acid sequence encoding a polypeptide associated with extracellular vesicles and/or AAV particles secreted from a cell.

Disclosed herein is a vector comprising an isolated nucleic acid molecule comprising a nucleic acid sequence encoding a polypeptide for promoting the formation of extracellular vesicles and/or AAV particles in a cell and at least one therapeutic agent.

Disclosed herein is a vector comprising an isolated nucleic acid molecule comprising a nucleic acid sequence encoding a polypeptide associated with extracellular vesicles and/or AAV particles secreted from a cell and at least one therapeutic agent.

Disclosed herein is a vector comprising an isolated nucleic acid molecule comprising a nucleic acid sequence encoding a polypeptide for promoting the formation of extracellular vesicles and/or AAV particles in a cell and an endonuclease.

Disclosed herein is a vector comprising an isolated nucleic acid molecule comprising a nucleic acid sequence encoding a polypeptide associated with extracellular vesicles and/or AAV particles secreted from a cell and an endonuclease.

Disclosed herein is a vector comprising an isolated nucleic acid molecule comprising a nucleic acid sequence encoding a fusion product, wherein the fusion product comprises a polypeptide for promoting the formation of extracellular vesicles and/or AAV particles secreted from a cell and at least one therapeutic agent.

Disclosed herein is a vector comprising an isolated nucleic acid molecule comprising a nucleic acid sequence encoding a fusion product, wherein the fusion product comprises a polypeptide associated with extracellular vesicles and/or AAV particles secreted from a cell and at least one therapeutic agent.

Disclosed herein is a vector comprising an isolated nucleic acid molecule comprising: a nucleic acid sequence encoding a fusion product, wherein the fusion product comprises a polypeptide for promoting the formation of extracellular vesicles and/or AAV particles secreted from a cell and an endonuclease.

Disclosed herein is a vector comprising an isolated nucleic acid molecule comprising a nucleic acid sequence encoding a fusion product, wherein the fusion product comprises a polypeptide associated with extracellular vesicles and/or AAV particles secreted from a cell and an endonuclease.

Disclosed herein is a pharmaceutical formulation comprising a disclosed vector in a pharmaceutically acceptable carrier.

Disclosed herein is a pharmaceutical formulation comprising a disclosed isolated nucleic acid molecule in a pharmaceutically acceptable carrier.

Disclosed herein is a pharmaceutical formulation comprising a disclosed fusion product in a pharmaceutically acceptable carrier.

Disclosed herein is a pharmaceutical formulation comprising secreted extracellular vesicles and/or AAV particles in a pharmaceutically acceptable carrier.

Disclosed herein is a method of enhancing secretion of extracellular vesicles and/or AAV particles from a cell comprising delivering to a cell an isolated nucleic acid molecule comprising a nucleic acid sequence encoding a polypeptide for promoting the formation of extracellular vesicles and/or AAV particles in a cell; expressing the encoded polypeptide; and secreting extracellular vesicles and/or AAV particles from the cell.

Disclosed herein is a method of enhancing secretion of extracellular vesicles and/or AAV particles from a cell comprising delivering to a cell an isolated nucleic acid molecule comprising a nucleic acid sequence encoding a polypeptide associated with extracellular vesicles and/or AAV particles secreted from a cell; expressing the encoded polypeptide; and secreting extracellular vesicles and/or AAV particles from the cell.

Disclosed herein is a method of delivering a therapeutic agent comprising delivering to a cell an isolated nucleic acid molecule comprising a nucleic acid sequence encoding a fusion product; expressing the encoded fusion product; encapsulating the encoded fusion product in one or more extracellular vesicles and/or AAV particles; and secreting extracellular vesicles and/or AAV particles from the cell.

Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide for promoting the formation of extracellular vesicles and/or AAV particles in a cell and at least one therapeutic agent, and expressing the encoded fusion product.

Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide associated with extracellular vesicles and/or AAV particles secreted from a cell and at least one therapeutic agent, and expressing the encoded fusion product.

Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide for promoting the formation of extracellular vesicles and/or AAV particles in a cell and an endonuclease, and expressing the encoded fusion product.

Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide associated with extracellular vesicles and/or AAV particles secreted from a cell and an endonuclease, and expressing the encoded fusion product.

Disclosed herein is a method of improving viral particle egress from a cell comprising delivering to a cell an isolated nucleic acid molecule comprising a nucleic acid sequence encoding (i) a polypeptide for promoting the formation of extracellular vesicles and/or AAV particles in cell or (ii) a polypeptide associated with extracellular vesicles and/or AAV particles secreted from a cell; expressing the encoded polypeptide; and encapsulating viral particles in one or more extracellular vesicles and/or AAV particles.

Disclosed herein is a method of altering or modifying the dynamics of extracellular vesicle and/or AAV particle formation and/or secretion from a cell, comprising delivering to a cell an isolated nucleic acid molecule comprising a nucleic acid sequence encoding (i) a polypeptide for promoting the formation of extracellular vesicles and/or AAV particles in cell or (ii) a polypeptide associated with extracellular vesicles and/or AAV particles secreted from a cell; and expressing the encoded polypeptide.

Disclosed herein is a method of altering or modifying the dynamics of extracellular vesicle and/or AAV particle formation and/or secretion from a cell, comprising delivering to a cell an isolated nucleic acid molecule comprising a nucleic acid sequence encoding a fusion product, wherein the fusion product encodes at least a polypeptide for promoting the formation of extracellular vesicles and/or AAV particles in cell or (ii) a polypeptide associated with extracellular vesicles and/or AAV particles secreted from a cell; and expressing the encoded polypeptide.

Disclosed herein is a method of loading extracellular vesicles and/or AAV particles with a cargo comprising delivering to a cell an isolated nucleic acid molecule comprising a nucleic acid sequence encoding a polypeptide for promoting the formation of extracellular vesicles and/or AAV particles, and expressing an encoded polypeptide, wherein the encoded polypeptide is directed to extracellular vesicles and/or AAV particles.

Disclosed herein is a method of loading extracellular vesicles and/or AAV particles with a cargo comprising delivering to a cell an isolated nucleic acid molecule comprising a nucleic acid sequence encoding a polypeptide associated with extracellular vesicles and/or AAV particles, and expressing an encoded polypeptide, wherein the encoded polypeptide is directed to an extracellular vesicle and/or AAV particle.

Disclosed herein is a method of loading extracellular vesicles with a cargo comprising delivering to a cell an isolated nucleic acid molecule comprising a nucleic acid sequence encoding a fusion product, expressing an encoded fusion product comprising (i) a polypeptide promoting the formation of extracellular vesicles and/or AAV particles in cell and (ii) cargo; wherein the fusion product is directed to an extracellular vesicle and/or AAV particles.

Disclosed herein is a method of loading extracellular vesicles with a cargo comprising delivering to a cell an isolated nucleic acid molecule comprising a nucleic acid sequence encoding a fusion product, expressing an encoded fusion product comprising (i) a polypeptide associated with extracellular vesicles and/or AAV particles secreted from a cell and (ii) cargo; wherein the fusion product is directed to an extracellular vesicle and/or an AAV particle.

### BRIEF DESCRIPTION OF THE FIGURES

FIG. 1A is a schematic of the WT AAV genome showing Rep and Cap genes with MAAP encoded in a +1 open reading frame in the VP1 region.

FIG. 1B shows a sequence alignment of the MAAPs from AAV serotypes 1 to 13 along with AAVrh.8 and AAVrh.10. An annotated multiple-sequence alignment of the AAP sequences of 15 AAV serotypes is shown. Coloring reflects the physicochemical properties of the residues (Yellow=hydrophobic, Green=polar, Blue=basic, Red=acidic). Regions of interest are annotated above the alignment. Predicted secondary structural (SS) elements (strand, helix) for the sequence and amino acid numbering are displayed below the alignments.

FIG. 1C shows structural models of MAAP1, MAAP2, MAAP5, MAAP8, and MAAP9 generated using Phyre² protein modeling software. Residues highlighted in blue indicate N-terminus and residues in red indicate C-terminus.

FIG. 1D shows neighbor-joining phylogeny of MAAP amino acid sequences from AAV serotypes 1 to 13, MAAPrh.8, and MAAPrh.10. MAAP amino acid sequences were aligned with ClustalW, the phylogeny was generated using a neighbor-joining algorithm, and a Poisson correction was used to calculate amino acid distances, represented as units of the number of amino acid substitutions per site. The tree is drawn to scale, with branch lengths in the same units as those of the evolutionary distances used to infer the tree. Bootstrap values were calculated with 1,000 replicates, and the percentage of replicate trees in which the associated taxa clustered together are shown next to the branches.

FIG. 1E shows an anti-GFP immunoblot of whole-cell extracts prepared from HEK293 cells expressing indicated GFP tagged constructs. An anti-actin immunoblot served as loading control.

FIG. 1F shows confocal images of HEK293 cells overexpressing eGFP tagged MAAP constructs. Scale bar=10 μM.

FIG. 1G shows the analysis of recombinant AAV8 and AAV8 MAAPΔ viral capsids by SDS-PAGE under reducing conditions and stained with coomassie following purification from the media of HEK293 producing cells.

FIG. 1H shows the analysis of recombinant AAV8 and AAV8 MAAPΔ viral capsids by SDS-PAGE under reducing conditions and probed with a capsid (B1) specific antibody following purification from the media of HEK293 producing cells.

FIG. 1I shows TEM images of rAAV8 viral capsids.

FIG. 1J shows TEM images of rAAV8 MAAPΔ viral capsids.

FIG. 2A shows a schematic of WT AAV8 MAAPΔ mutant.

FIG. 2B shows the total vector genomes collected from the cells and media of cells producing AAV8 ssCBA-Luc vectors with WT cap or MAAPΔ cap.

FIG. 2C shows the proportion of virus found in each media harvest or associated with the cells producing AAV8 ssCBA-Luc vectors with WT cap or MAAPΔ cap.

FIG. 2D shows a schematic of rAAV8 MAAPΔ mutant.

FIG. 2E shows the total vector genomes collected from the cells and media of cells producing AAV8 ssCBA-Luc vectors with recombinant cap or MAAPΔ cap.

FIG. 2F shows the proportion of virus found in each media harvest or associated with the cells producing AAV8 ssCBA-Luc vectors with recombinant cap or MAAPΔ cap.

FIG. 2G shows the analysis of recombinant AAV8 and AAV8 MAAPΔ viruses from the media and pellet of HEK293 producing cells at day 3 post-infection post-transfection. Capsid proteins were analyzed by SDS-PAGE under reducing conditions and probed with a capsid (B1) specific antibody.

FIG. 2H shows the analysis of recombinant AAV8 and AAV8 MAAPΔ viruses from the media and pellet of HEK293 producing cells at day 5 post-infection. Capsid proteins were analyzed by SDS-PAGE under reducing conditions and probed with a capsid (B1) specific antibody.

FIG. 2I shows a luciferase assay analyzing transduction of HEK293 cells by AAV8 and AAV8 MAAPΔ mutant virus at MOIs of 10,000 and 50,000 vg/cell. Each bar is a representation of three experiments that are biological replicates. Error bars indicate standard deviation from the mean. Significance was determined by two-way ANOVA, with Sidak's post-test. *p<0.05. **p<0.01, ***p<0.001, ****p<0.0001.

FIG. 2J shows the total vector genomes collected from the cells and media of cells producing AAV9 ssCBA-Luc vectors with WT cap or MAAPΔ cap. Each bar is a representation of three experiments that are biological replicates. Error bars indicate standard deviation from the mean. Significance was determined by two-way ANOVA, with Tukey's post-test. ns=not significant.

FIG. 2K shows the proportion of virus found in each media harvest or associated with the cells producing AAV9 ssCBA-Luc vectors with WT cap or MAAPΔ cap.

FIG. 2L shows that recombinant AAV9 and AAV9 MAAPΔ viruses were analyzed from the media and pellet of HEK293 producing cells at days 3 and 5 post-transfection. Capsid proteins were analyzed by SDS-PAGE under reducing conditions and probed with a capsid (B1) specific antibody.

FIG. 3A shows the sequence alignment of MAAP8 (SEQ ID NO:08) with different MAAP mutants (SEQ ID NO:36 SEQ ID NO:49), with all MAAP mutants having a 3×-FLAG tag at the C terminus.

FIG. 3B shows anti-FLAG immunoblot of whole-cell extracts prepared from HEK293 cells expressing indicated MAAP8-3×-FLAG tagged constructs with anti-actin immunoblot served as loading control.

FIG. 3C shows anti-FLAG immunoblot of whole-cell extracts prepared from HEK293 cells expressing additional indicated MAAP8-3×-FLAG tagged constructs with anti-actin immunoblot served as loading control.

FIG. 3D shows recombinant MAAP8Δ vectors complemented in trans with various truncated MAAP8-3×-FLAG plasmids analyzed from the media and pellet of HEK293 producing cells at day 3 post-transfection. Capsid proteins were analyzed by SDS-PAGE under reducing conditions and probed with a capsid (B1) specific antibody.

FIG. 3E shows recombinant MAAP8Δ vectors complemented in trans with additional various truncated MAAP8-3×-FLAG plasmids analyzed from the media and pellet of HEK293 producing cells at day 3 post-transfection. Capsid proteins were analyzed by SDS-PAGE under reducing conditions and probed with a capsid (B1) specific antibody.

FIG. 3F shows the total vector genomes found in the media and cells 3 days post-transfection. Each bar is a representation of three experiments that are biological replicates. Error bars indicate standard deviation from the mean.

FIG. 3G shows the proportion of vector found in the media and cells 3 days post-transfection. Each bar is a representation of three experiments that are biological replicates. Error bars indicate standard deviation from the mean.

FIG. 3H shows a schematic of the recombinant AAV8 VP/AAP-null MAAP8-3×-FLAG (MAAP8-3×-FLAG) plasmid used to replicate endogenous levels of MAAP expression.

FIG. 3I shows immunoblots of the whole cell lysate of HEK293 cells transfected with MAAP8-3×-FLAG along with pXX680 (Adenoviral helper) plasmids and harvested 72 hours post transfection, which lysates were analyzed by SDS-PAGE under reducing conditions and probed with FLAG (α-FLAG) and actin (α-actin) specific antibodies.

FIG. 3J shows immunoblots of recombinant AAV8 and AAV8 MAAPΔ viruses complemented with MAAP8-3×-FLAG analyzed from the media of HEK293 producing cells at day 3 post transfection. Capsid proteins were analyzed by SDS-PAGE under reducing conditions and probed with a capsid (B1) specific antibody.

FIG. 4A shows total vector genomes of scCBh-GFP vectors produced with WT Cap or MAAPΔ Cap for AAV1 3 days post-transfection and shows rAAV1 complemented in trans with a VP/AAP-null AAV8 plasmid replicated endogenous levels of MAAP expression.

FIG. 4B shows the proportion of scCBh-GFP vectors produced with WT Cap or MAAPΔ Cap for AAV1 found in the media and in the cells 3 days post-transfection and shows rAAV1 complemented in trans with a VP/AAP-null AAV8 plasmid replicated endogenous levels of MAAP expression.

FIG. 4C shows total vector genomes of scCBh-GFP vectors produced with WT Cap or MAAPΔ Cap for AAV2 3 days post-transfection and shows rAAV2 complemented in trans with a VP/AAP-null AAV8 plasmid to replicate endogenous levels of MAAP expression.

FIG. 4D shows the proportion of scCBh-GFP vectors produced with WT Cap or MAAPΔ Cap for AAV2 found in the media and in the cells 3 days post-transfection and shows rAAV2 complemented in trans with a VP/AAP-null AAV8 plasmid replicated endogenous levels of MAAP expression.

FIG. 4E shows total vector genomes of scCBh-GFP vectors produced with WT Cap or MAAPΔ Cap for rAAV8 3 days post-transfection and shows rAAV8 complemented in trans with a VP/AAP-null AAV8 plasmid replicated endogenous levels of MAAP expression.

FIG. 4F shows the proportion of scCBh-GFP vectors produced with WT Cap or MAAPΔ Cap for rAAV8 found in the media and in the cells 3 days post-transfection and shows rAAV8 complemented in trans with a VP/AAP-null AAV8 plasmid replicated endogenous levels of MAAP expression.

FIG. 4G shows total vector genomes of scCBh-GFP vectors produced with WT Cap or MAAPΔ Cap for rAAV9 3 days post-infection and shows rAAV9 complemented in trans with a VP/AAP-null AAV8 plasmid replicated endogenous levels of MAAP expression.

FIG. 4H shows the proportion of scCBh-GFP vectors produced with WT Cap or MAAPΔ Cap for rAAV9 found in the media and in the cells 3 days post-infection and shows rAAV9 complemented in trans with a VP/AAP-null AAV8 plasmid replicated endogenous levels of MAAP expression. For FIGS. 4A-4H, each bar is a representation of three experiments that are biological replicates. Error bars indicate standard deviation from the mean. Significance was determined by two-way ANOVA, with Tukey's post-test. *p<0.05, **p<0.01, ***p<0.001, ****p<0.0001.

FIG. 4I shows the anti-HA immunoblot of whole-cell extracts prepared from HEK293 cells expressing the indicated HA tagged constructs with anti-actin immunoblot served as loading control.

FIG. 5A shows HEK293 cells transfected with expression vectors encoding Rab7-GFP (top row, second panel from the left), Rab11-GFP (bottom row, second panel from the left), and MAAP8-HA (top and bottom rows, second panel from the right) as well as a merged image (right most panel). MAAP-HA was detected by immunofluorescence with an AlexaFlour647 secondary antibody (MAAP8-HA-A647). A Z-stack of confocal optical sections at 1-μm steps was acquired. A 3-μm-thick medial stack is shown. Images are representative of three experiments. Scale bars, 10 μm.

FIG. 5B shows the co-localization between MAAP8-HA and Rab7-GFP or Rab11-GFP in the whole cell as assessed by Pearson's correlation coefficient (R) as described above. Each dot represents one cell. Horizontal bars represent the mean±SEM, Mann-Whitney rank test. (****p<0.0001, ^{μs}p≥0.05).

FIG. 5C shows the analysis of exosomes isolated from media of AAV8 and AAV8 MAAPΔ producing HEK293 cells and analyzed by SDS-PAGE under reducing conditions and probed with an anti-capsid monoclonal antibody (B1).

FIG. 5D shows the analysis of exosomes isolated from media of AAV8 and AAV8 MAAPΔ producing HEK293 cells and analyzed by SDS-PAGE under reducing conditions and probed with exosome (α-CD81) specific antibody.

FIG. 5E shows HEK293 cells transfected with expression vectors encoding HA and MAAP8-HA, exosomes were then isolated from media 72 hours post-transfection, analyzed by SDS-PAGE under reducing conditions, and probed with an exosome (α-CD81) specific antibody.

FIG. 5F shows HEK293 cells transfected with expression vectors encoding GFP and MAAP8-GFP, exosomes were then isolated from the media 72 hours post-transfection, analyzed by SDS-PAGE under reducing conditions, and probed with a GFP (α-GFP) specific antibody.

FIG. 5G show TEM images of exosomes isolated from media of HEK293 cells producing recombinant AAV8 MAAPΔ that were transfected with an expression vector encoding HA.

FIG. 5H shows TEM images of exosomes isolated from media of HEK293 cells producing recombinant AAV8 MAAPΔ that were transfected with an expression vector encoding MAAP8-HA. In FIG. 5G-5H, highlighted top inset region magnified in bottom image, left-scale bars represent 200 nm, middle-scale bars represent 100 nm, and right-scale bars represent 50 nm.

FIG. 5I shows HEK293 cells transfected with expression vectors encoding Rab7-GFP (top row, second panel from the left). Rab11-GFP (bottom row, second panel from the left), and MAAP9-HA (top and bottom rows, second panel from the right) as well as a merged image (right most panels). MAAP-HA was detected by immunofluorescence with an AlexaFlour647 secondary antibody (MAAP9-HA-A647). A Z-stack of confocal optical sections at 1 μm steps was acquired. A 3-μm-thick medial stack is shown. Images are representative of three experiments. Scale bars, 10 μm.

FIG. 5J shows the co-localization between MAAP9-HA and Rab7-GFP or Rab11-GFP in the whole cell as assessed by Pearson's correlation coefficient (R) as described above. Each dot represents one cell. Horizontal bars represent the mean±SEM, Mann-Whitney rank test. ^{ns}p≥0.05.

FIG. 6A shows the immunoprecipitation (IP) of MAAP8/9-HA with rAAV8 and rAAV9 capsids and immunoblotting of input whole cell lysate (WCL) and pull down (PD) material for actin, capsid (B1), and MAAP8/9-HA.

FIG. 6B shows the immunoprecipitation of AAP1-C9 and MAAP1-HA and immunoblotting of input whole cell lysate (WCL) and pull down (PD) material for actin, capsid (B1), MAAP8/9 (HA), and AAP (ID4).

FIG. 7A shows a schematic of MAAP8-13×-BioID2-HA fusions.

FIG. 7B shows whole cell lysate (WCL) analyzed by SDS-PAGE under reducing conditions and probed with HA (α-HA), biotin (α-biotin), and actin (α-actin) specific antibodies of harvested HEK293 transfected with expression vectors encoding 13×-BiolD2 and MAAP8-13×-BioID2. Here, media was supplemented with 50 μM biotin 24 hours post-transfection and cells were harvested 24 hours post-biotin supplementation.

FIG. 7C shows biotinylated proteins pulled down on streptavidin resin, which were separated by SDS-PAGE and visualized by silver stain, from harvested HEK293 cells transfected with plasmids encoding either 13×-BioID2 or MAAP8-13×-BiolD2 along with pXX680, pTR-CBA-Luciferase, and AAV8-MAAPΔ. Media was supplemented with 50 μM biotin 48 hours post-transfection and cells were harvested 20 hours post-biotin supplementation.

FIG. 7D shows biotinylated proteins pulled down on streptavidin resin, which were separated by SDS-PAGE and probed with biotin (α-biotin), (α-HA), and capsid (B1) specific antibodies, from harvested HEK293 cells transfected with plasmids encoding either 13×-BiolD2 or MAAP8-13×-BioID2 along with pXX680, pTR-CBA-Luciferase, and AAV8-MAAPΔ. Media was supplemented with 50 μM biotin 48 hours post-transfection and cells were harvested 20 hours post-biotin supplementation.

FIG. 8A shows a schematic for a Cas9-HA fusion product and a schematic for MAAP8-Cas9-HA fusion product.

FIG. 8B shows an anti-Cas9-HA immunoblot of whole-cell lysates prepared from HEK293 cells expressing the Cas9-HA fusion construct and the MAAP8-Cas9-HA fusion construct.

FIG. 9A shows a schematic highlighting the methodology utilized for exosome isolation and characterization.

FIG. 9B shows anti-CD81, anti-CD63, anti-CD9, and anti-Cas9-HA immunoblots of individual iodixanol fractions from the conditioned media of HEK239 cells transfected with SaCas9.

FIG. 9C shows anti-CD81, anti-CD63, anti-CD9, and anti-Cas9-HA immunoblots of individual iodixanol fractions from the conditioned media of HEK239 cells transfected with MAAP8-SaCas9.

FIG. 9D shows the quantitative analysis of exosomal and Cas9 markers in individual iodixanol fractions of conditioned media of SaCas9-HA. Signal intensity normalized to maximum intensity of each individual marker.

FIG. 9E shows the quantitative analysis of exosomal and Cas9 markers in individual iodixanol fractions of conditioned media of MAAP8-SaCas9-HA. Signal intensity normalized to maximum intensity of each individual marker.

FIG. 10A shows a schematic highlighting the downstream processing of exosome containing iodixanol fractions.

FIG. 10B shows anti-CD81, anti-CD63, and anti-Cas9-HA immunoblots of individual processed iodixanol fractions from the conditioned media of HEK239 cells transfected with either SaCas9 or MAAP8-SaCas9.

FIG. 10C shows the quantitative analysis of exosomal and Cas9 markers in individual processed iodixanol fractions for SaCas9-HA, which demonstrated a strong association between exosomal and SaCas9-HA markers in fraction 2, thereby indicating loading of MAAP8-Cas9 into exosomes. Signal intensity normalized to maximum intensity of each individual marker.

FIG. 10C shows the quantitative analysis of exosomal and Cas9 markers in individual processed iodixanol fractions for MAAP8-SaCas9-HA, which demonstrated a strong association between exosomal and Cas9-HA markers in fraction 2, thereby indicating loading of MAAP8-Cas9 into exosomes. Signal intensity normalized to maximum intensity of each individual marker.

FIG. 11 provides a schematic showing how MAAP-AA V particles as provided herein are incorporated and secreted by a cell.

### DETAILED DESCRIPTION OF THE INVENTION

The present disclosure describes compositions, isolated nucleic acids, fusion products, pharmaceutical formulations, and methods of using the disclosed compositions, isolated nucleic acids, fusion products, pharmaceutical formulations thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, vectors, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then I1, 12, 13, and 14 are also disclosed.

As used herein, the term “approximately” or “about,” as applied to one or more values of interest, refers to a value that is similar to a stated reference value. In an aspect, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction of the stated reference value unless otherwise stated or otherwise evident from the context.

As used herein, the term “in vitro” refers to events or experiments that occur in an artificial environment, e.g., in a petri dish, test tube, cell culture, etc., rather than within a multicellular organism. As used herein, the term “in vivo” refers to events or experiments that occur within a multicellular organism.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, the term “subject” refers to the target of administration. In an aspect, a subject can be a human being. The term “subject” includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease, a disorder, an infection, a symptom, and/or a complication, be suspected of having a disease, a disease, a disorder, an infection, a symptom, and/or a complication, or be at risk of developing a disease, a disorder, an infection, a symptom, and/or a complication. For example, a subject can have risk factors for developing a disease, a disorder, an infection, a symptom, and/or a complication. Risk factors can include, but are not limited to the following: cancer, chronic kidney disease, chronic obstructive pulmonary disease, an immunocompromised state (weakened immune system) from solid organ transplant, obesity (body mass index [BMI] of 30 or higher), serious heart conditions (e.g., heart failure, coronary artery disease, or cardiomyopathies), sickle cell disease, diabetes mellitus, asthma (moderate-to-severe), cerebrovascular disease (i.e., disease that affects blood vessels and blood supply to the brain), cystic fibrosis, hypertension or high blood pressure, immunocompromised state (weakened immune system) from blood or bone marrow transplant, immune deficiencies. HIV, use of corticosteroids, or use of other immune weakening medicines, neurologic conditions (e.g. dementia, Alzheimer's), liver disease, pregnancy, pulmonary fibrosis (having damaged or scarred lung tissues), tobacco use, smoking, thalassemia. A subject can be at risk due to genetic predisposition, employment type (e.g., a health care worker, a miner), attendance at a specific location (e.g., school), attendance at social events (e.g., sporting events, concerns, religious services, political rallies and events, social justice rallies, marches, and events, etc.), by use of public transportation or public services, exposure to natural and man-made disasters (e.g., Chernobyl, 9/11 attacks, etc.).

In an aspect, a subject can have a genetic disorder. Genetic disorders include but are not limited to Cystic fibrosis, Hurler Syndrome, alpha-1-antitrypsin (A1AT) deficiency, Parkinson's disease, Alzheimer's disease, albinism, Amyotrophic lateral sclerosis. Asthma, Thalassemia, Cadasil syndrome, Charcot-Marie-Tooth disease, Chronic Obstructive Pulmonary Disease (COPD), Distal Spinal Muscular Atrophy (DSMA), Duchenne/Becker muscular dystrophy, Dystrophic Epidermolysis bullosa, Epidermylosis bullosa, Fabry disease. Factor V Leiden associated disorders, Familial Adenomatous, Polyposis, Galactosemia. Gaucher's Disease, Glucose-6-phosphate dehydrogenase, Haemophilia, Hereditary Hematochromatosis, Hunter Syndrome, Huntington's disease, Inflammatory Bowel Disease (IBD), Inherited polyagglutination syndrome, Leber congenital amaurosis, Lesch-Nyhan syndrome, Lynch syndrome, Marfan syndrome, Mucopolysaccharidosis, Muscular Dystrophy, Myotonic dystrophy types I and II, neurofibromatosis, Niemann-Pick disease type A, B and C. NY-esol related cancer, Peutz-Jeghers Syndrome, Phenylketonuria, Pompe's disease, Primary Ciliary Disease, Prothrombin mutation related disorders, such as the Prothrombin G20210A mutation, Pulmonary Hypertension, Retinitis Pigmentosa, Sandhoff Disease, Severe Combined Immune Deficiency Syndrome (SCID), Sickle Cell Anemia, Spinal Muscular Atrophy, Stargardt's Disease, Tay-Sachs Disease. Usher syndrome, X-linked immunodeficiency, and cancer.

In an aspect, a subject can have cancer. Cancer includes, but is not limited to, ovarian cancer, epithelial ovarian cancer, non-Hodgkin's lymphomas (such as diffuse large B-cell lymphoma), acute myeloid leukemia, thymus cancer, brain cancer, lung cancer, squamous cell cancer, skin cancer, eye cancer, retinoblastoma, intraocular melanoma, oral cavity and oropharyngeal cancer, bladder cancer, gastric cancer, stomach cancer, pancreatic cancer, breast cancer, cervical cancer, head and neck cancer, renal cancer, kidney cancer, liver cancer, prostate, colorectal cancer, bone (e.g., metastatic bone), esophageal cancer, testicular cancer, gynecological cancer, thyroid cancer, central nervous system lymphomas, AIDS-related cancers (e.g., lymphoma and Kaposi's sarcoma), viral-induced cancers such as cervical carcinoma (human papillomavirus). B-cell lymphoproliferative disease and nasopharyngeal carcinoma (Epstein-Barr virus), Kaposi's sarcoma and primary effusion lymphomas, hepatocellular carcinoma (hepatitis B and hepatitis C viruses), and T-cell leukemias (human T-cell leukemia virus-1), B cell acute lymphoblastic leukemia, Burkitt's leukemia, juvenile myelomonocytic leukemia, hairy cell leukemia. Hodgkin's disease, multiple myeloma, mast cell leukemia, and mastocytosis.

As used herein, “effective amount” and “amount effective” can refer to an amount that is sufficient to achieve the desired result such as, for example, the treatment and or prevention of a disease, a disorder, an infection, a symptom, and/or a complication, or a suspected disease, disorder, infection, symptom, and/or complication. As used herein, the terms “effective amount” and “amount effective” can refer to an amount that is sufficient to achieve the desired effect on an undesired disease, disorder, infection, symptom, and/or complication. For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed composition that (i) treats the particular disease, disorder, and/or infection, (ii) attenuates, ameliorates, or eliminates one or more symptoms of the particular disease, condition, and/or disorder, or (iii) delays the onset of one or more symptoms of the particular disease, condition, and/or disorder described herein. The specific therapeutically effective dose level for any particular patient will depend upon a variety of factors including the disorder being treated and the severity of the disorder; the specific disclosed compositions and/or a pharmaceutical preparation comprising one or more disclosed compositions, or methods employed; the age, body weight, general health, sex and diet of the patient; the time of administration; the route of administration; the rate of excretion of the disclosed compositions and/or a pharmaceutical preparation comprising one or more disclosed compositions employed; the duration of the treatment; drugs used in combination or coincidental with a disclosed compositions and/or a pharmaceutical preparation comprising one or more disclosed compositions employed, and other like factors well known in the medical arts. For example, it is well within the skill of the art to start doses of a disclosed composition and/or a pharmaceutical preparation comprising one or more disclosed composition at levels lower than those required to achieve the desired therapeutic effect and to gradually increase the dosage until the desired effect is achieved. If desired, then the effective daily dose can be divided into multiple doses for purposes of administration. Consequently, a single dose of a disclosed compositions and/or a pharmaceutical preparation comprising one or more disclosed compositions, or methods can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease, a disorder, an infection, a symptom, and/or a complication.

“Control” as used herein refers a standard or reference condition, against which results are compared. In an aspect, a control is used at the same time as a test variable or subject to provide a comparison. In an aspect, a control is a historical control that has been performed previously, a result or amount that has been previously known, or an otherwise existing record. A control may be a positive or negative control.

As used herein, the term “diagnosed” means having been subjected to a physical examination by a person of skill, for example, a physician, and found to have a disease, a disorder, an infection, a symptom, and/or a complication that can be diagnosed or treated by one or more of the disclosed nucleic acids, the disclosed vectors, the disclosed fusion products, the disclosed compositions, the disclosed pharmaceutical preparations, and/or the disclosed methods. For example, “suspected of having” can mean having been subjected to a physical examination by a person of skill, for example, a physician, and found to have a condition that can likely be treated by one or more of the disclosed nucleic acids, the disclosed vectors, the disclosed fusion products, the disclosed compositions, the disclosed pharmaceutical preparations, and/or the disclosed methods.

The words “treat” or “treating” or “treatment” refer to therapeutic or medical treatment wherein the object is to slow down (lessen), ameliorate, and/or diminish an undesired physiological change, disease, pathological condition, or disorder in a subject. As used herein, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Treatment may not necessarily result in the complete clearance of an infection but may reduce or minimize complications, the side effects, and/or the progression of a disease, a disorder, an infection, a symptom, and/or a complication. The success or otherwise of treatment may be monitored by physical examination of the subject as well as cytopathological, DNA, and/or mRNA detection techniques. The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating an infection can reduce the severity of an established infection in a subject by 1%-100% as compared to a control (such as, for example, a subject not having the disease, the disorder, the infection, the symptom, and/or the complication. In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of an established disease, disorder, infection, symptom, and/or complication. In an aspect, treating can refer to 1%, 2%, 3%, 4%, 5%, 6%, 7%, 8%, 9%, 10%, 20%, 30%, 40%. 50%, 60%, 70%. 80%, 90%, 100% reduction of one or more symptoms. It is understood that treatment does not necessarily refer to a cure or complete ablation or eradication of the disease, disorder, infection, symptom, and/or complication. However, in an aspect, treatment can refer to a cure or complete ablation or eradication of the disease, disorder, infection, symptom, and/or complication.

Methods and techniques to monitor a subject's response to a disclosed method can comprise qualitative (or subjective) means as well as quantitative (or objective) means. In an aspect, qualitative means (or subjective means) can comprise a subject's own perspective. For example, a subject can report how he/she is feeling, whether he/she has experienced improvements and/or setbacks, whether he/she has experienced an amelioration or an intensification of one or more symptoms, or a combination thereof. In an aspect, quantitative means (or objective means) can comprise methods and techniques that include, but are not limited to, the following: (i) fluid analysis (e.g., tests of a subject's fluids including but not limited to aqueous humor and vitreous humor, bile, blood, blood serum, breast milk, cerebrospinal fluid, cerumen (earwax), digestive fluids, endolymph and perilymph, female ejaculate, gastric juice, mucus (including nasal drainage and phlegm), peritoneal fluid, pleural fluid, saliva, sebum (skin oil), semen, sweat, synovial fluid, tears, vaginal secretion, vomit, and urine), (ii) imaging (e.g., ordinary x-rays, ultrasonography, radioisotope (nuclear) scanning, computed tomography (CT), magnetic resonance imaging (MRI), positron emission tomography (PET), and angiography), (iii) endoscopy (e.g., laryngoscopy, bronchoscopy, esophagostomy, gastroscopy, GI endoscopy, coloscopy, cystoscopy, hysteroscopy, arthroscopy, laparoscopy, mediastinoscopy, and thoracoscopy), (iv) analysis of organ activity (e.g., electrocardiography (ECG), electroencephalography (EEG), and pulse oximetry), (v) biopsy (e.g., removal of tissue samples for microscopic evaluation), and (vi) genetic testing.

A “patient” refers to a subject afflicted with a disease, disorder, infection, symptom, and/or complication. In an aspect, a patient can refer to a subject that has been diagnosed with or is suspected of having a disease, disorder, infection, symptom, and/or complication. In an aspect, a patient can refer to a subject that has been diagnosed with or is suspected of having an established disease, disorder, infection, symptom, and/or complication and is seeking treatment or receiving treatment.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease, disorder, infection, symptom, and/or complication is intended. The words “prevent” and “preventing” and “prevention” also refer to prophylactic or preventative measures for protecting or precluding a subject (e.g., an individual) not having a given infection related complication from progressing to that complication. Individuals in which prevention is required include those who have an infection.

As used herein, the terms “administering” and “administration” refer to any method of providing one or more of the disclosed nucleic acids, the disclosed vectors, the disclosed fusion products, the disclosed compositions, and/or the disclosed pharmaceutical preparations to a subject. Such methods are well known to those skilled in the art and include, but are not limited to, the following: oral administration, transdermal administration, administration by inhalation, nasal administration, topical administration, intravaginal administration, ophthalmic administration, intraaural administration, otic administration, inter utero administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent.

As used herein, a “targeting moiety” can be specific to a recognition molecule on the surface of a target cell or a target population of cells, such as, for example B-cells or a type of cancer cell. In an aspect of the disclosed compositions and disclosed methods, a targeting moiety can include, but is not limited to a monoclonal antibody, a polyclonal antibody, full-length antibody, a chimeric antibody, Fab′, Fab, F(ab)2, F(ab′)2, a single domain antibody (DAB), Fv, a single chain Fv (scFv), a minibody, a diabody, a triabody, hybrid fragments, a phage display antibody, a ribosome display antibody, a peptide, a peptide ligand, a hormone, a growth factor, a cytokine, a saccharide or polysaccharide, and an aptamer. A targeting moiety can be specific for a specific type of cell such as smooth or striatal muscle cells, lung cells, kidney cells, skin cells, heart cells, liver cells, brain cells, pancreatic cells, or any other target cell type. A targeting moiety can be specific for a specific type of cell such as a cancer cell.

As used herein, “extracellular vesicle uptake” or “EV uptake” refers to the interaction of one or more EVs with a target cell. In an aspect, EVs can bind to the cell surface via antigen-antibody interaction or ligand-receptor interactions and can potentially trigger signaling via surface receptors, even without EV entry into the cell. As known to the art, the most common mode of EV uptake into target cells involves internalization via endocytotic processes, such as clathrin, caveolin, or lipid raft-mediated endocytosis, micropinocytosis, or phagocytosis. In an aspect, EVs can also directly fuse with the plasma membrane of the cell and release the encapsulated cargo directly into the cytoplasm. In an aspect, EVs can comprise AAV particles.

As used herein, “modifying the method” can comprise modifying or changing one or more features or aspects of one or more steps of a disclosed method. For example, in an aspect, a method can be altered by changing the amount of one or more of the disclosed nucleic acids, the disclosed vectors, the disclosed fusion products, the disclosed compositions, and/or the disclosed pharmaceutical preparations administered to a subject, or by changing the frequency of administration, or by changing the duration of time of administration or between administrations to a subject.

As used herein, “concurrently” means (1) simultaneously in time, or (2) at different times during the course of a common treatment schedule.

The term “contacting” as used herein refers to bringing one or more of the disclosed nucleic acids, the disclosed vectors, the disclosed fusion products, the disclosed compositions, and/or the disclosed pharmaceutical preparations together with a target area or intended target area in such a manner that the one or more disclosed nucleic acids, vectors, fusion products, compositions, and/or pharmaceutical preparation can exert an effect on the intended target or targeted area either directly or indirectly. In an aspect, secreted EVs can contact one or more nearby or surrounding cells. In an aspect, secreted EVs can contact one or more target cells or one or more target populations of cells.

As used herein, “determining” can refer to measuring or ascertaining the presence and severity of a disease, disorder, infection, symptom, and/or complication. Methods and techniques used to determining the presence and/or severity of a disease, disorder, infection, symptom, and/or complication are typically known to the medical arts. For example, the art is familiar with the ways to identify and/or diagnose the presence, severity, or both of a disease, disorder, infection, symptom, and or complication.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, tale, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, “CRISPR or clustered regularly interspaced short palindromic repeat” is an ideal tool for correction of genetic abnormalities as the system can be designed to target genomic DNA directly. A CRISPR system involves two main components: a Cas9 enzyme and a guide (gRNA). The gRNA contains a targeting sequence for DNA binding and a scaffold sequence for Cas9 binding. Cas9 nuclease is often used to “knockout” target genes hence it can be applied for deletion or suppression of oncogenes that are essential for cancer initiation or progression. Similar to ASOs and siRNAs, CRISPR offers a great flexibility in targeting any gene of interest hence, potential CRISPR based therapies can be designed based on the genetic mutation in individual patients. An advantage of CRISPR is its ability to completely ablate the expression of disease genes which can only be suppressed partially by RNA interference methods with ASOs or siRNAs. Furthermore, multiple gRNAs can be employed to suppress or activate multiple genes simultaneously, hence increasing the treatment efficacy and reducing resistance potentially caused by new mutations in the target genes.

As used herein, “CRISPR-based endonucleases” include RNA-guided endonucleases that comprise at least one nuclease domain and at least one domain that interacts with a guide RNA. As known to the art, a guide RNA directs the CRISPR-based endonucleases to a targeted site in a nucleic acid at which site the CRISPR-based endonucleases cleaves at least one strand of the targeted nucleic acid sequence. As the guide RNA provides the specificity for the targeted cleavage, the CRLSPR-based endonuclease is universal and can be used with different guide RNAs to cleave different target nucleic acid sequences. CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al. (2012) Science. 337:816-821).

In an aspect, a disclosed CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cul966.

In an aspect, a disclosed CRISPR-based endonuclease can be derived from a type II CRISPR/Cas system. For example, in an aspect, a CRISPR-based endonuclease can be derived from a Cas9 protein. The Cas9 protein can be from Streptococcus pyogenes, Streptococcus thermophilus, Streptococcus sp, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thennopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In an aspect, the CRISPR-based nuclease can be derived from a Cas9 protein from Streptococcus pyogenes.

In general, CRISPR/Cas proteins can comprise at least one RNA recognition and/or RNA binding domain. RNA recognition and/or RNA binding domains can interact with the guide RNA such that the CRISPR/Cas protein is directed to a specific genomic or genomic sequence. CRISPR/Cas proteins can also comprise nuclease domains (i.e., DNase or RNase domains). DNA binding domains, helicase domains, protein-protein interaction domains, dimerization domains, as well as other domains.

The CRISPR-based endonuclease can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, in an aspect, nuclease (i.e., DNase, RNase) domains of the CRISPR/Cas protein can be modified, deleted, or inactivated. A CRISPR/Cas protein can be truncated to remove domains that are not essential for the function of the protein. A CRISPR/Cas protein also can be truncated or modified to optimize the activity of the protein or an effector domain fused with a CRISPR/Cas protein.

In an aspect, a disclosed CRISPR-based endonuclease can be derived from a wild type Cas9 protein or fragment thereof. In an aspect, a disclosed CRISPR-based endonuclease can be derived from a modified Cas9 protein. For example, the amino acid sequence of a disclosed Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

As used herein, the term “derivative” refers to a compound having a structure derived from the structure of a parent compound (such as, e.g., a polypeptide having the sequence set forth in any of SEQ ID NOS:01-15, 33, or 35-49 or a nucleic acid having the sequence set forth in any of SEQ ID NOS:16-30 and 34) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds. Exemplary derivatives include fragments of a disclosed protein (e.g., SEQ ID NOS:01-15, 33, or 35-49) or nucleic acid sequence (e.g., SEQ ID NOS:16-30 and 34).

As used herein, the term “analog” refers to a compound having a structure derived from the structure of a parent compound (such as, e.g., a polypeptide having the sequence set forth in any of SEQ ID NOS:01-15, 33, or 35-49 or a nucleic acid having the sequence set forth in any of SEQ ID NOS:16-30 and 34) and whose structure is sufficiently similar to those disclosed herein and based upon that similarity, would be expected by one skilled in the art to exhibit the same or similar activities and utilities as the claimed compounds, or to induce, as a precursor, the same or similar activities and utilities as the claimed compounds.

As used herein, “extracellular vesicles” (EVs) is a generic term that can refer to all membrane vesicles secreted in the extracellular space. As such, EVs include a broad and extremely heterogeneous population of vesicles, which possess different functions, biophysical properties, and have different biogenesis routes. Given the lack of a clear consensus on the nomenclature of EVs, the field has coined a multitude of terms to address the different types of vesicles, resulting in sub-categories that are often redundant and/or overlapping. Accordingly, the terms “ectosomes”, “shedding vesicles”, “microvesicles”, and “microparticles” usually refer to 150-1000 nm vesicles that bud directly from the plasma membrane, while the term “exosomes” refers to smaller vesicles (30-100 nm), which are generated intracellularly by the inward budding of multivesicular bodies (MVB) and released in the extracellular space upon fusion of the MVBs with the plasma membrane. EVs can package different macromolecules including proteins, nucleic acids and viruses, thereby making them an attractive therapeutic platform. (Pegtel D M, et al. 2019 Exosomes. Annu Rev Biochem. 88:487-514; Colombo M. et al. 2014 Annu Rev Cell Dev Biol. 30:255-289). Relevant to the disclosed compositions and methods, recombinant AAV capsids associated with exosomes can enable efficient gene transfer to the retina, the nervous system, the inner ear (Hudry E, et al. 2016 Gene Ther. 23(4):380-392; György B, et al. 2017 Mol Ther. 25(2):379-391; Meliani A, et al. 2017 Blood Adv. 1(23):2019-2031; Volak A, et al. 2018 J Neurooncol. 139(2):293-305) and appear shielded from anti-AAV neutralizing antibodies. (Meliani A, et al. 2017 Blood Adv. 1(23):2019-2031).

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Neuron-specific promoters” are known to the art and include, but are not limited to, the synapsin I (SYN) promoter, the calcium/calmodulin-dependent protein kinase II promoter, the tubulin alpha I promoter, the neuron-specific enolase promoter, and the platelet-derived growth factor beta chain promoter.

“Liver-specific promoters” are known to the art and include, but are not limited to, the α1-microglobulin/bikunin enhancer/thyroid hormone-binding globulin promoter, the human albumin (hALB) promoter, the thyroid hormone-binding globulin promoter, thyroxin binding globulin promoter, the α-1-anti-trypsin promoter, the bovine albumin (bAlb) promoter, the murine albumin (mAlb) promoter, the human a1-antitrypsin (hAAT) promoter, the ApoEhAAT promoter composed of the ApoE enhancer and the hAAT promoter, the transthyretin (TTR) promoter, the liver fatty acid binding protein promoter, the hepatitis B virus (HBV) promoter, the DC172 promoter consisting of the hAAT promoter and the a1-microglobulin enhancer, the DC190 promoter containing the human albumin promoter and the prothrombin enhancer, and other natural and synthetic liver-specific promoters.

“Muscle-specific promoters” are known to the art and include, but are not limited to, the MHCK7 promoter, the muscle creatine kinase (MCK) promoter/enhancer, the slow isoform of troponin I (TnIS) promoter, the MYOD1 promoter, the MYLK2 promoter, the SPc5-12 promoter, the desmin (Des) promoter, the unc45b promoter, and other natural and synthetic muscle-specific promoters.

“Skeletal muscle-specific promoters” are known to the art and include, but are not limited to, the HSA promoter, the human α-skeletal actin promoter.

“Heart-specific promoters” are known to the art and include, but art not limited to, the MYH6 promoter, the TNNI3 promoter, the cardiac troponin C (cTnC) promoter, the alpha-myosin heavy chain (α-MHC) promoter, myosin light chain 2 (MLC-2), and the MYBPC3 promoter.

As used herein, the term “immunotolerant” refers to unresponsiveness to an antigen (e.g., a vector, a therapeutic protein derived from a human, a non-human animal, a plant, or a microorganism, such as, for example, a microbial GBE. An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, a “ubiquitous/constitutive promoter” refer to a promoter that allows for continual transcription of its associated gene. A ubiquitous-constitutive promoter is always active and can be used to express genes in a wide range of cells and tissues, including, but not limited to, the liver, kidney, skeletal muscle, cardiac muscle, smooth muscle, diaphragm muscle, brain, spinal cord, endothelial cells, intestinal cells, pulmonary cells (e.g., smooth muscle or epithelium), peritoneal epithelial cells, and fibroblasts. Ubiquitous/constitutive promoters include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, an “isolated” biological component (such as a nucleic acid molecule, protein, or virus) has been substantially separated or purified away from other biological components (e.g., other chromosomal and extra-chromosomal DNA and RNA, proteins and/or organelles). Nucleic acids, proteins, and/or viruses that have been “isolated” include nucleic acids, proteins, and viruses purified by standard purification methods. The term also embraces nucleic acids, proteins, and viruses prepared by recombinant expression in a host cell, as well as chemically synthesized nucleic acids or proteins. The term “isolated” (or purified) does not require absolute purity; rather, it is intended as a relative term. Thus, for example, an isolated or purified nucleic acid, protein, virus, or other active compound is one that is isolated in whole or in part from associated nucleic acids, proteins, and other contaminants. In an aspect, the term “substantially purified” refers to a nucleic acid, protein, virus or other active compound that has been isolated from a cell, cell culture medium, or other crude preparation and subjected to fractionation to remove various components of the initial preparation, such as proteins, cellular debris, and other components.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%. 99% or more. Such sequences are also referred to as “variants” herein, e.g., other variants of glycogen branching enzymes and amylases. It should be understood that sequence with substantial sequence identity do not necessarily have the same length and may differ in length. For example, sequences that have the same nucleotide sequence but of which one has additional nucleotides on the 3′- and/or 5′-side are 00% identical.

Codon-optimized: A “codon-optimized” nucleic acid refers to a nucleic acid sequence that has been altered such that the codons are optimal for expression in a particular system (such as a particular species or group of species). For example, a nucleic acid sequence can be optimized for expression in mammalian cells or in a particular mammalian species (such as human cells). Codon optimization does not alter the amino acid sequence of the encoded protein.

Disclosed are the components to be used to prepare one or more of the disclosed nucleic acids, the disclosed vectors, the disclosed fusion products, the disclosed compositions, and/or the disclosed pharmaceutical preparations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D. E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions

### 1. Membrane-Associated Accessory Proteins (MAAP)

Disclosed herein is a membrane-associated protein (MAAP) derived from an alternate reading frame in the genome sequence of an Adeno-Associated Virus (AAV), wherein MAAP promotes the formation of extracellular vesicles and/or AAV particles in a mammalian cell; and wherein MAAP comprises the sequence set forth in any one of SEQ ID NO. 01-SEQ ID NO:15. Disclosed herein is a membrane-associated accessory protein (MAAP) derived from an alternate reading frame in the genome sequence of an Adeno-Associated Virus (AAV), wherein MAAP associates with extracellular vesicles and/or AAV particles secreted from a mammalian cell; and wherein MAAP comprises the sequence set forth in any one of SEQ ID NO:01-SEQ ID NO:15. Disclosed herein is a membrane-associated accessory protein (MAAP) comprising the sequence set forth in any one of SEQ ID NO:01-SEQ ID NO:15, wherein MAAP comprises an N-terminal domain connected to a C-terminal cationic, amphipathic membrane anchoring domain through a linker domain. Disclosed herein is a membrane-associated accessory protein (MAAP) derived from an alternate reading frame in the genome sequence of an Adeno-Associated Virus (AAV), wherein MAAP promotes the formation of extracellular vesicles and/or AAV particles in a mammalian cell; and wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15. Disclosed herein is a membrane-associated accessory protein (MAAP) derived from an alternate reading frame in the genome sequence of an Adeno-Associated Virus (AAV), wherein MAAP associates with extracellular vesicles and/or AAV particles secreted from a mammalian cell; and wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15. Disclosed herein is a membrane-associated accessory protein (MAAP) comprising the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed membrane-associated accessory protein (MAAP) can comprise a sequence having at least 30%, at least 35%, at least 40%, at least 45%, at least 50%, at least 55%, at least 60%, at least 65% b, at least 70%, at least 75%, at least 80%, at least 85%, at least 90%, or at least 95% identity to the sequence set forth in SEQ ID NO:08 or a fragment thereof. In an aspect, a disclosed membrane-associated accessory protein (MAAP) can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed membrane-associated accessory protein (MAAP) can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed membrane-associated accessory protein (MAAP) can be covalently or non-covalently attached to one or more of a polypeptide, a glycopeptide, a polysaccharide, a glycolipid, a lipid, a nucleic acid polymer, or is covalently attached to a combination thereof. In an aspect, the disclosed polypeptides, glycopeptides, polysaccharides, glycolipids, lipids, nucleic acid polymers, or the combinations thereof can be therapeutic.

In an aspect, a disclosed membrane-associated accessory protein (MAAP) can be covalently or non-covalently attached to one or more therapeutic agents.

In an aspect, a disclosed therapeutic agent can comprise an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed oligonucleotide therapeutic agent can be a CRISPR-based endonuclease.

In an aspect, a disclosed membrane-associated accessory protein (regardless of whether MAAP is covalently or non-covalently attached to another molecule or complex) can be encapsulated in one or more extracellular vesicles and/or AAV particles, wherein the one more or more extracellular vesicles and/or AAV particles can be secreted by the cell. In an aspect, a disclosed membrane-associated accessory protein (regardless of whether MAAP is covalently or non-covalently attached to another molecule or complex) can be encapsulated in one or more nanoparticles, wherein the one more or more nanoparticles can be secreted by the cell. In an aspect, disclosed nanoparticles can be encapsulated in disclosed extracellular vesicles and/or AAV particles.

In an aspect, a disclosed membrane-associated accessory protein (MAAP) can be covalently attached or non-covalently attached to an AAV capsid. In an aspect, the MAAP-AAV capsid complex can be encapsulated in extracellular vesicles and/or AAV particles, wherein the one more or more extracellular vesicles and/or AAV particles can be secreted by the cell.

### 2. Capsid Gene Sequences

Disclosed herein is an AAV capsid gene sequence comprising the sequence set forth in any one of SEQ ID NO:16-SEQ ID NO:30, wherein the sequence encodes a membrane-associated accessory protein (MAAP) when read in an alternate reading frame. Table 2 shows the serotype for each of SEQ ID NO:16-SEQ ID NO:30. Table 4 provides the nucleotide sequence for each of SEQ ID NO:16-SEQ ID NO:30.

In an aspect, the encoded membrane-associated accessory protein (MAAP) of a disclosed AAV capsid can comprise the sequence set forth in any one of SEQ ID NO:01-SEQ ID NO:15. Table 1 shows the serotype for each of SEQ ID NO:01-SEQ ID NO:15. Table 4 provides the amino acid sequence for each of SEQ ID NO:01-SEQ ID NO:15.

In an aspect, a disclosed membrane-associated accessory protein (MAAP) can associate with extracellular vesicles and/or AAV particles secreted from a cell. In an aspect, a disclosed membrane-associated accessory protein (MAAP) can promote the formation of extracellular vesicles and/or AAV particles in a cell.

In an aspect, a disclosed membrane-associated accessory protein (MAAP) can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed cell can be a mammalian cell or a non-mammalian cell. In an aspect, a disclosed cell can be a eukaryotic cell or a prokaryotic cell. In an aspect, a disclosed cell can be a human cell. In an aspect, a disclosed cell can be in a subject. In an aspect, a disclosed subject can be a human or a non-human primate. In an aspect, a disclosed cell can be in culture.

Disclosed herein is a modified AAV capsid gene sequence comprising the sequence set forth in any one of SEQ ID NO:16 SEQ ID NO:30, wherein the sequence comprises one or more modifications; and wherein the one or more modifications alters a cell's ability to secrete extracellular vesicles and/or AAV particles. In an aspect, the one or more modifications can be at any position of the sequence. In an aspect, the cell's altered ability comprises the amount of extracellular vesicles and/or AAV particles secreted by the cell. In an aspect, the cell's altered ability can comprise the rate of formation of extracellular vesicles and/or AAV particles. In an aspect, a disclosed cell can be a mammalian cell or a non-mammalian cell. In an aspect, a disclosed cell can be a eukaryotic cell or a prokaryotic cell. In an aspect, a disclosed cell can be a human cell. In an aspect, a disclosed cell can be in a subject. In an aspect, a disclosed subject can be a human or a non-human primate. In an aspect, a disclosed cell can be in culture.

### 3. Isolated Nucleic Acid Molecules

Disclosed herein is an isolated nucleic acid molecule comprising a nucleic acid sequence encoding a polypeptide for promoting the formation of extracellular vesicles and/or AAV particles in cell. Disclosed herein is an isolated nucleic acid molecule comprising a nucleic acid sequence encoding a polypeptide associated with extracellular vesicles and/or AAV particles secreted from a cell.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain.

In an aspect, a disclosed encoded polypeptide can modulate the rate or efficiency of extracellular vesicle and/or AAV particle secretion. Modulate can comprise increasing the rate or efficiency of extracellular vesicle and/or AAV particle secretion, or modulate can comprise decreasing the rate or efficiency of extracellular vesicle and/or AAV particle secretion.

In an aspect, a disclosed encoded polypeptide can alter or modify the dynamics of AAV particle secretion. In an aspect, altering or modifying the dynamics of AAV particle secretion can comprise increasing the rate of particle secretion, increasing the rate of particle formation, or both.

In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed MAAP can be the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. Table 1 shows the serotype for each of SEQ ID NOS:01-15.

TABLE 1
SEQ ID NO. and Serotype (Amino Acids)

| SEQ ID NO. | Serotype |
|---|---|
| SEQ ID NO: 01 | AAV1 |
| SEQ ID NO: 02 | AAV2 |
| SEQ ID NO: 03 | AAV3 |
| SEQ ID NO: 04 | AAV4 |
| SEQ ID NO: 05 | AAV5 |
| SEQ ID NO: 06 | AAV6 |
| SEQ ID NO: 07 | AAV7 |
| SEQ ID NO: 08 | AAV8 |
| SEQ ID NO: 09 | AAV9 |
| SEQ ID NO: 10 | AAV10 |
| SEQ ID NO: 11 | AAV11 |
| SEQ ID NO: 12 | AAV12 |
| SEQ ID NO: 13 | AAV13 |
| SEQ ID NO: 14 | AAVrh8 |
| SEQ ID NO: 15 | AAVrh10 |

In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 600, at least 65%, at least 70%, at least 75%, at least 80%, at least 85%, at least 90%, or at least 95% identity to the sequence set forth in SEQ ID NO:08. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ TID NO:09, SEQ ID NO:10, SEQ ID NO: 1, SEQ ID NO:12, SEQ ID NO:13, SEQ ID NO:14, SEQ ID NO:15.

In an aspect, a disclosed nucleic acid sequence can have the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof. In an aspect, a disclosed nucleic acid sequence can have a sequence having at least 30%, at least 40%, at least 50%, at least 60%, at least 70%, at least 80%, or at least 90% identity to the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ TID NO:29, SEQ ID NO:30, or a fragment thereof.

In an aspect, a disclosed nucleic acid for a MAAP can be a derivative or an analog of the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In an aspect, a disclosed nucleic acid for a MAAP can comprise the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ TID NO:25, SEQ ID NO:26, SEQ ID NO:27, SEQ ID NO:28, SEQ ID NO:29, and SEQ ID NO:30, wherein the sequence can comprise one or more mutations. In an aspect, the one or more mutations can affect the functionality of the encoded MAAP.

TABLE 2
SEQ ID NO. and Serotype (Nucleic Acids)

| SEQ ID NO. | Serotype |
|---|---|
| SEQ ID NO: 16 | AAV1 |
| SEQ ID NO: 17 | AAV2 |
| SEQ ID NO: 18 | AAV3 |
| SEQ ID NO: 19 | AAV4 |
| SEQ ID NO: 20 | AAV5 |
| SEQ ID NO: 21 | AAV6 |
| SEQ ID NO: 22 | AAV7 |
| SEQ ID NO: 23 | AAV8 |
| SEQ ID NO: 24 | AAV9 |
| SEQ ID NO: 25 | AAV10 |
| SEQ ID NO: 26 | AAV11 |
| SEQ ID NO: 27 | AAV12 |
| SEQ ID NO: 28 | AAV13 |
| SEQ ID NO: 29 | AAVrh8 |
| SEQ ID NO: 30 | AAVrh10 |

Disclosed herein is an isolated nucleic acid molecule comprising a nucleic acid sequence encoding a polypeptide for promoting the formation of extracellular vesicles and/or AAV particles in a cell and at least one therapeutic agent. Disclosed herein is an isolated nucleic acid molecule comprising a nucleic acid sequence encoding a polypeptide associated with extracellular vesicles and/or AAV particles secreted from a cell and at least one therapeutic agent. Disclosed herein is an isolated nucleic acid molecule comprising a nucleic acid sequence encoding a polypeptide for promoting the formation of extracellular vesicles and/or AAV particles in a cell and an endonuclease. Disclosed herein is an isolated nucleic acid molecule comprising a nucleic acid sequence encoding a polypeptide associated with extracellular vesicles and/or AAV particles secreted from a cell and an endonuclease.

In an aspect, a disclosed encoded polypeptide can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed encoded polypeptide can be a membrane-associated accessory protein (MAAP) or a fragment thereof. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. Table 1 shows the serotype for each of SEQ ID NOS:01-15.

In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08. In an aspect, a disclosed MAAP can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ TID NO:15.

In an aspect, a disclosed nucleic acid sequence can have the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof. In an aspect, a disclosed nucleic acid sequence can have a sequence having at least 30%, at least 40%, at least 50%, at least 60%, at least 70%, at least 80%, or at least 90% identity to the sequence set forth in SEQ ID NO:16, SEQ ID NO:17, SEQ ID NO:18, SEQ ID NO:19, SEQ TID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

In an aspect, a disclosed nucleic acid for a MAAP can be a derivative or an analog of the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In an aspect, a disclosed nucleic acid for a MAAP can comprise the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, wherein the sequence can comprise one or more mutations. In an aspect, the one or more mutations can affect the functionality of the encoded MAAP.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or to a combination thereof.

In an aspect, a disclosed polypeptide (e.g., MAAP) can be covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

Disclosed herein is an isolated nucleic acid molecule comprising a nucleic acid sequence encoding a fusion product, wherein the fusion product comprises a polypeptide for promoting the formation of extracellular vesicles and/or AAV particles secreted from a cell and at least one therapeutic agent. Disclosed herein is an isolated nucleic acid molecule comprising a nucleic acid sequence encoding a fusion product, wherein the fusion product comprises a polypeptide associated with extracellular vesicles and/or AAV particles secreted from a cell and at least one therapeutic agent. Disclosed herein is an isolated nucleic acid molecule comprising a nucleic acid sequence encoding a fusion product, wherein the fusion product comprises a polypeptide for promoting the formation of extracellular vesicles and/or AAV particles secreted from a cell and an endonuclease. Disclosed herein is an isolated nucleic acid molecule comprising a nucleic acid sequence encoding a fusion product, wherein the fusion product comprises a polypeptide associated with extracellular vesicles and/or AAV particles secreted from a cell and an endonuclease.

In an aspect, a disclosed fusion product can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and or secretion pathway.

In an aspect, a disclosed encoded polypeptide can be a membrane-associated accessory protein (MAAP) or a fragment thereof. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11. SEQ ID NO:12, SEQ ID NO:13, SEQ ID NO:14, SEQ ID NO:15, or a fragment thereof. Table 1 shows the serotype for each of SEQ ID NOS:01-15. In an aspect, a disclosed MAAP can have a sequence having at least 30%, at least 40′%, at least 50%, at least 60%, at least 700%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14. SEQ ID NO:15, ora fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ TID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15.

In an aspect, a disclosed nucleic acid sequence can have the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof. In an aspect, a disclosed nucleic acid sequence can have a sequence having at least 30%, at least 40%, at least 50%, at least 60%, at least 70%, at least 80%, or at least 90% identity to the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ TID NO:27, SEQ ID NO:28, SEQ ID NO:29, SEQ ID NO:30, or a fragment thereof.

In an aspect, a disclosed nucleic acid for a MAAP can be a derivative or an analog of the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In an aspect, a disclosed nucleic acid for a MAAP can comprise the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, wherein the sequence can comprise one or more mutations. In an aspect, the one or more mutations can affect the functionality of the encoded MAAP.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

### 4. Fusion Products

Disclosed herein is a fusion product comprising a polypeptide for promoting the formation of extracellular vesicles and/or AAV particles in a cell and at least one therapeutic agent. Disclosed herein is a fusion product comprising a polypeptide associated with extracellular vesicles and/or AAV particles secreted from a cell and at least one therapeutic agent. Disclosed herein is a fusion product comprising a polypeptide for promoting the formation of extracellular vesicles and/or AAV particles in a cell and an endonuclease. Disclosed herein is a fusion product comprising a polypeptide associated with extracellular vesicles and/or AAV particles secreted from a cell and an endonuclease.

In an aspect, a disclosed fusion product can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a polypeptide of a disclosed fusion product can be a membrane-associated accessory protein (MAAP) or a fragment thereof. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ TID NO:09, SEQ ID NO:10, SEQ ID NO:11, SEQ ID NO:12, SEQ ID NO:13, SEQ ID1 NO:14, SEQ ID NO:15, or a fragment thereof. Table 1 shows the serotype for each of SEQ ID NOS:01-15. In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise aa CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

In an aspect, a disclosed fusion product can localize to extracellular vesicles and/or AAV particles secreted from a cell. In an aspect, AAV particles can localize to extracellular vesicles secreted from a cell. In an aspect, the disclosed extracellular vesicles and/or AAV particles can encapsulate AAV particles. In an aspect, the disclosed extracellular vesicles and/or AAV particles can encapsulate a disclosed polypeptide. In an aspect, the disclosed extracellular vesicles and/or AAV particles can encapsulate a disclosed polypeptide covalently or non-covalently attached to one or more of a polypeptide, a glycopeptide, a polysaccharide, a glycolipid, a lipid, or a nucleic acid polymer, or a combination thereof. In an aspect, the disclosed extracellular vesicles and/or AAV particles can encapsulate one or more therapeutic agents. In an aspect, the disclosed extracellular vesicles and/or AAV particles can encapsulate a disclosed polypeptide covalently or non-covalently attached to one or more disclosed therapeutic agents. In an aspect, the disclosed extracellular vesicles and/or AAV particles can encapsulate one or more disclosed therapeutic agents.

In an aspect, a disclosed fusion product can be administered via intravenous, intraarterial, intramuscular, intraperitoneal, subcutaneous, intrathecal, intraventricular, or in utero administration. In an aspect, a disclosed fusion product can be administered via LNP administration. In an aspect, a disclosed fusion product can be delivered to a subject's liver, heart, skeletal muscle, smooth muscle, CNS, PNS, or a combination thereof.

### 5. Vectors

Disclosed herein is a vector comprising an isolated nucleic acid molecule comprising a nucleic acid sequence encoding a polypeptide for promoting the formation of extracellular vesicles and/or AAV particles in cell. Disclosed herein is a vector comprising an isolated nucleic acid molecule comprising a nucleic acid sequence encoding a polypeptide associated with extracellular vesicles and/or AAV particles secreted from a cell. Disclosed herein is a vector comprising an isolated nucleic acid molecule comprising a nucleic acid sequence encoding a polypeptide for promoting the formation of extracellular vesicles and/or AAV particles in a cell and at least one therapeutic agent. Disclosed herein is a vector comprising an isolated nucleic acid molecule comprising a nucleic acid sequence encoding a polypeptide associated with extracellular vesicles and/or AAV particles secreted from a cell and at least one therapeutic agent. Disclosed herein is a vector comprising an isolated nucleic acid molecule comprising a nucleic acid sequence encoding a polypeptide for promoting the formation of extracellular vesicles and/or AAV particles in a cell and an endonuclease. Disclosed herein is a vector comprising an isolated nucleic acid molecule comprising a nucleic acid sequence encoding a polypeptide associated with extracellular vesicles and/or AAV particles secreted from a cell and an endonuclease.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain.

In an aspect, a disclosed encoded polypeptide can modulate the rate or efficiency of extracellular vesicle secretion and/or AAV particles. Modulate can comprise increasing the rate or efficiency of extracellular vesicle and/or AAV particle secretion, or modulate can comprise decreasing the rate or efficiency of extracellular vesicle and/or AAV particle secretion.

In an aspect, a disclosed encoded polypeptide can alter or modify the dynamics of AAV particle secretion. In an aspect, altering or modifying the dynamics of AAV particle secretion can comprise increasing the rate of particle secretion, increasing the rate of particle formation, or both.

In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed MAAP can be the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof.

In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO: 13, SEQ ID NO: 14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or covalently attached to a combination thereof.

In an aspect, a disclosed polypeptide (e.g., MAAP) can be non-covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

In an aspect, a disclosed vector can be a viral vector or a non-viral vector.

In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed non-viral vector can be a polymer based vector, a peptide based vector, a lipid nanoparticle, a solid lipid nanoparticle, or a cationic lipid based vector.

In an aspect, a disclosed vector can comprise one or more regulatory elements. A disclosed vector can comprise a ubiquitous promoter operably linked to a disclosed isolated nucleic acid molecule, wherein the ubiquitous promoter drives the expression of a disclosed encoded polypeptide, a disclosed encoded therapeutic agent, or both. A disclosed vector can comprise a tissue specific promoter operably linked to a disclosed isolated nucleic acid molecule, wherein the tissue specific promoter drives the expression of a disclosed encoded polypeptide, a disclosed encoded therapeutic agent, or both. A disclosed vector can comprise an immunotolerant dual promoter comprising a tissue-specific promoter and a ubiquitous promoter.

The nucleic acid sequence of a disclosed vector can have a coding sequence that is less than about 4.5 kilobases.

Disclosed herein is a vector comprising an isolated nucleic acid molecule comprising a nucleic acid sequence encoding a fusion product, wherein the fusion product comprises a polypeptide for promoting the formation of extracellular vesicles and/or AAV particles secreted from a cell and at least one therapeutic agent. Disclosed herein is a vector comprising an isolated nucleic acid molecule comprising a nucleic acid sequence encoding a fusion product, wherein the fusion product comprises a polypeptide associated with extracellular vesicles and/or AAV particles secreted from a cell and at least one therapeutic agent. Disclosed herein is a vector comprising an isolated nucleic acid molecule comprising: a nucleic acid sequence encoding a fusion product, wherein the fusion product comprises a polypeptide for promoting the formation of extracellular vesicles and/or AAV particles secreted from a cell and an endonuclease. Disclosed herein is a vector comprising an isolated nucleic acid molecule comprising a nucleic acid sequence encoding a fusion product, wherein the fusion product comprises a polypeptide associated with extracellular vesicles and/or AAV particles secreted from a cell and an endonuclease.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or covalently attached to a combination thereof.

In an aspect, a disclosed polypeptide (e.g., MAAP) can be non-covalently attached or non-covalently attached to one or more of a polypeptide, a glycopeptide, a polysaccharide, a glycolipid, a lipid, or a nucleic acid polymer, or non-covalently attached to a combination thereof.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%1, at least 90%, or at least 95% identity to the sequence set forth in SEQ ID NO:34 or a fragment thereof.

In an aspect, a disclosed vector can be a viral vector or a non-viral vector. In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh10, AAV11, AAV12, AAV13. AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed non-viral vector can be a polymer based vector, a peptide based vector, a lipid nanoparticle, a solid lipid nanoparticle, or a cationic lipid based vector.

In an aspect, a disclosed vector can comprise one or more regulatory elements. A disclosed vector can comprise a ubiquitous promoter operably linked to a disclosed isolated nucleic acid molecule, wherein the ubiquitous promoter drives the expression of a disclosed encoded polypeptide, a disclosed encoded therapeutic agent, or both. A disclosed vector can comprise a tissue specific promoter operably linked to a disclosed isolated nucleic acid molecule, wherein the tissue specific promoter drives the expression of a disclosed encoded polypeptide, a disclosed encoded therapeutic agent, or both. A disclosed vector can comprise an immunotolerant dual promoter comprising a tissue-specific promoter and a ubiquitous promoter. The nucleic acid sequence of a disclosed vector can have a coding sequence that is less than about 4.5 kilobases.

### 6. Pharmaceutical Formulations

Disclosed herein is a pharmaceutical formulation comprising a disclosed vector in a pharmaceutically acceptable carrier. Disclosed herein is a pharmaceutical formulation comprising a disclosed nucleic acid molecule in a pharmaceutically acceptable carrier. Disclosed herein is a pharmaceutical formulation comprising a disclosed fusion product in a pharmaceutically acceptable carrier.

### 7. Kits

Disclosed herein is a kit comprising one or more disclosed compositions. In an aspect, a composition of a disclosed kit can comprise a disclosed isolated nucleic acid molecule, a disclosed fusion product, a disclosed vector, a disclosed pharmaceutical composition, or a combination thereof. In an aspect, a disclosed kit can comprise a combination of one or more active agents. In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed composition and a label or package insert with instructions for use. In an aspect, a kit can contain one or more additional agents (e.g., excipients, buffers, active agents, biologically active agents, pharmaceutically active agents, immune-based therapeutic agents, clinically approved agents, or a combination thereof). In an aspect, one or more active agents can treat, inhibit, and or ameliorate one or more comorbidities in a subject. In an aspect, one or more active agents can treat, inhibit, and/or ameliorate a disease, a disorder, an infection, a symptom, a complication, or a combination thereof. In an aspect, suitable containers include, for example, bottles, vials, syringes, blister pack, etc. The containers can be formed from a variety of materials such as glass or plastic. The container can hold a disclosed composition or a pharmaceutical formulation comprising a disclosed composition and can have a sterile access port (for example the container may be an intravenous solution bag or a vial having a stopper pierceable by a hypodermic injection needle). The label or package insert can indicate that a disclosed composition or a pharmaceutical formulation comprising a disclosed composition can be used for treating, preventing, inhibiting, and/or ameliorating a disease, a disorder, an infection, a symptom, a complication, or a combination thereof. A kit can comprise additional components necessary for administration such as, for example, other buffers, diluents, filters, needles, and syringes. The term “package insert” can refer to instructions customarily included in commercial packages of therapeutic products, that contain information about the indications, usage, dosage, administration, contraindications and/or warnings concerning the use of such therapeutic products.

### C. Methods

1. Methods of Enhancing Secretion of Extracellular Vesicles and/or AAV Particles

Disclosed herein is a method of enhancing secretion of extracellular vesicles and/or AAV particles from a cell comprising delivering to a cell an isolated nucleic acid molecule comprising a nucleic acid sequence encoding a polypeptide for promoting the formation of extracellular vesicles and/or AAV particles in a cell; expressing the encoded polypeptide; and secreting extracellular vesicles and/or AAV particles from the cell. Disclosed herein is a method of enhancing secretion of extracellular vesicles and/or AAV particles from a cell comprising delivering to a cell an isolated nucleic acid molecule comprising a nucleic acid sequence encoding a polypeptide associated with extracellular vesicles and/or AAV particles secreted from a cell; expressing the encoded polypeptide; and secreting extracellular vesicles and/or AAV particles from the cell.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain.

In an aspect, a disclosed encoded polypeptide can modulate the rate or efficiency of extracellular vesicle secretion. Modulate can comprise increasing the rate or efficiency of extracellular vesicle secretion, or modulate can comprise decreasing the rate or efficiency of extracellular vesicle secretion.

In an aspect, a disclosed encoded polypeptide can alter or modify the dynamics of AAV particle secretion. In an aspect, altering or modifying the dynamics of AAV particle secretion can comprise increasing the rate of particle secretion, increasing the rate of particle formation, or both.

In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. Table 1 shows the serotype for each of SEQ ID NOS:01-15. In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46. SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15.

In an aspect, a disclosed nucleic acid sequence can have the sequence set forth in SEQ ID NO:16, SEQ ID NO:17, SEQ ID NO:18, SEQ ID NO:19, SEQ TID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof. Table 2 shows the serotype for each of SEQ ID NOS:16-30. In an aspect, a disclosed nucleic acid sequence can have a sequence having at least 30%, at least 40%, at least 50%, at least 60%, at least 70%, at least 80%, or at least 90% identity to the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

In an aspect, a disclosed nucleic acid for a MAAP can be a derivative or an analog of the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In an aspect, a disclosed nucleic acid for a MAAP can comprise the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, wherein the sequence can comprise one or more mutations. In an aspect, the one or more mutations can affect the functionality of the encoded MAAP.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or to a combination thereof. In an aspect, a disclosed polypeptide (e.g., MAAP) can be covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, delivering a disclosed isolated nucleic acid molecule can comprise using a vector. In an aspect, a disclosed vector can be a viral vector or a non-viral vector. In an aspect, a disclosed vector can comprise one or more regulatory elements.

In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed non-viral vector can be a polymer based vector, a peptide based vector, a lipid nanoparticle, a solid lipid nanoparticle, or a cationic lipid based vector.

In an aspect, a disclosed cell can be a mammalian cell or a non-mammalian cell or a eukaryotic cell or a prokaryotic cell. In an aspect, a disclosed cell can be a human cell. In an aspect, a disclosed cell can be in a subject. In an aspect, a subject can be a human or a non-human primate. In an aspect, a disclosed cell can be in culture.

In an aspect, a disclosed method can comprise harvesting the secreted extracellular vesicles and/or AAV particles from conditioned media of the culture. In an aspect, expressing the encoded polypeptide can comprise transient expression or stable expression. In an aspect of a disclosed method, an encoded polypeptide can localize to extracellular vesicles and/or AAV particles secreted from a cell. In an aspect of a disclosed method. AAV particles can localize to extracellular vesicles secreted from a cell. In an aspect of a disclosed method, AAV particles can be secreted from a cell. In an aspect, the disclosed extracellular vesicles can encapsulate AAV particles. In an aspect, the disclosed extracellular vesicles and/or AAV particles can encapsulate a disclosed polypeptide. In an aspect, the disclosed extracellular vesicles and/or AAV particles can encapsulate a disclosed polypeptide covalently or non-covalently attached to one or more of a polypeptide, a glycopeptide, a polysaccharide, a glycolipid, a lipid, or a nucleic acid polymer, or a combination thereof. In an aspect, the disclosed extracellular vesicles and/or AAV particles can encapsulate one or more therapeutic agents. In an aspect, the disclosed vesicles can encapsulate a disclosed polypeptide covalently or non-covalently attached to one or more disclosed therapeutic agents. In an aspect of a disclosed method, secreted extracellular vesicles and/or AAV particles can comprise one or more targeting moieties. Targeting moieties are known to the art.

### 2. Methods of Delivering a Therapeutic Agent

Disclosed herein is a method of delivering a therapeutic agent comprising delivering to a cell an isolated nucleic acid molecule comprising a nucleic acid sequence encoding a fusion product; expressing the encoded fusion product; encapsulating the encoded fusion product in one or more extracellular vesicles and/or AAV particles; and secreting extracellular vesicles and/or AAV particles from the cell.

In an aspect of a disclosed method, a fusion product can comprise a polypeptide for promoting the formation of extracellular vesicles and/or AAV particles in a cell and at least one therapeutic agent. In an aspect of a disclosed method, a fusion product can comprise a polypeptide associated with extracellular vesicles and/or AAV particles secreted from a cell and at least one therapeutic agent. In an aspect of a disclosed method, a fusion product can comprise a polypeptide for promoting the formation of extracellular vesicles and/or AAV particles in a cell and an endonuclease. In an aspect of a disclosed method, a fusion product can comprise a polypeptide associated with extracellular vesicles and/or AAV particles secreted from a cell and an endonuclease.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain.

In an aspect, a disclosed encoded polypeptide can alter or modify the dynamics of AAV particle secretion. In an aspect, altering or modifying the dynamics of AAV particle secretion can comprise increasing the rate of particle secretion, increasing the rate of particle formation, or both. In an aspect, altering or modifying the dynamics of AAV particle secretion can comprise decreasing the rate of particle secretion, decreasing the rate of particle formation, or both. In an aspect, altering or modifying the dynamics of AAV particle secretion can comprise affecting one or more aspects of the AAV particle formation and/or secretion pathway.

In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. Table 1 shows the serotype for each of SEQ ID NOS:01-15. In an aspect, a disclosed MAAP can have a sequence having at least 30%, at least 40%, at least 50%, at least 60%, at least 70% N, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ TID NO:10, SEQ ID NO:11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or to a combination thereof.

In an aspect, a disclosed polypeptide (e.g., MAAP) can be non-covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

In an aspect of a disclosed method, an encoded polypeptide can localize to extracellular vesicles and/or AAV particles secreted from a cell. In an aspect of a disclosed method, AAV particles can localize to extracellular vesicles secreted from a cell. In an aspect, the disclosed extracellular vesicles can encapsulate AAV particles. In an aspect, the disclosed extracellular vesicles and/or AAV particles can encapsulate a disclosed polypeptide. In an aspect, the disclosed extracellular vesicles and/or AAV particles can encapsulate a disclosed polypeptide covalently or non-covalently attached to one or more of a polypeptide, a glycopeptide, a polysaccharide, a glycolipid, a lipid, or a nucleic acid polymer, or a combination thereof. In an aspect, the disclosed extracellular vesicles and/or AAV particles can encapsulate one or more therapeutic agents. In an aspect, the disclosed extracellular vesicles and/or AAV particles can encapsulate a disclosed polypeptide covalently or non-covalently attached to one or more disclosed therapeutic agents. In an aspect, the disclosed extracellular vesicles and/or AAV particles can encapsulate one or more disclosed therapeutic agents. In an aspect of a disclosed method, secreted extracellular vesicles and/or AAV particles can comprise one or more targeting moieties. In an aspect of a disclosed method, secreted extracellular vesicles and/or AAV particles can contact one or more other cells. In an aspect of a disclosed method, an encoded polypeptide can localize to extracellular vesicles and/or AAV particles secreted from a cell.

In an aspect, a disclosed cell can be a mammalian cell or a non-mammalian cell or a eukaryotic cell or a prokaryotic cell. In an aspect, a disclosed cell can be a human cell. In an aspect, a disclosed cell can be in a subject. In an aspect, a subject can be a human or a non-human primate. In an aspect, a disclosed cell can be in culture.

In an aspect, expressing the encoded polypeptide can comprise transient expression or stable expression.

In an aspect, a disclosed vector can be a viral vector or a non-viral vector. In an aspect, a disclosed non-viral vector can be a polymer based vector, a peptide based vector, a lipid nanoparticle, a solid lipid nanoparticle, or a cationic lipid based vector.

In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5. AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed vector can comprise one or more regulatory elements. A disclosed vector can comprise a ubiquitous promoter operably linked to a disclosed isolated nucleic acid molecule, wherein the ubiquitous promoter drives the expression of a disclosed encoded polypeptide, a disclosed encoded therapeutic agent, or both. A disclosed vector can comprise a tissue specific promoter operably linked to a disclosed isolated nucleic acid molecule, wherein the tissue specific promoter drives the expression of a disclosed encoded polypeptide, a disclosed encoded therapeutic agent, or both. A disclosed vector can comprise an immunotolerant dual promoter comprising a tissue-specific promoter and a ubiquitous promoter. The nucleic acid sequence of a disclosed vector can have a coding sequence that is less than about 4.5 kilobases.

Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide for promoting the formation of extracellular vesicles and/or AAV particles in a cell and at least one therapeutic agent, and expressing the encoded fusion product. Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide associated with extracellular vesicles and/or AAV particles secreted from a cell and at least one therapeutic agent, and expressing the encoded fusion product. Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide for promoting the formation of extracellular vesicles and/or AAV particles in a cell and an endonuclease, and expressing the encoded fusion product. Disclosed herein is a method of delivering a therapeutic agent to a subject comprising administering to a subject a vector comprising an isolated nucleic acid molecule comprising a nucleic acid sequence encoding a fusion product, wherein the fusion product comprises a polypeptide associated with extracellular vesicles and/or AAV particles secreted from a cell and an endonuclease, and expressing the encoded fusion product.

In an aspect of a disclosed method, secreted extracellular vesicles and/or AAV particles can comprise one or more targeting moieties. Targeting moieties are known to the art.

In an aspect, the disclosed extracellular vesicles can encapsulate AAV particles. In an aspect, the disclosed extracellular vesicles can encapsulate a disclosed polypeptide. In an aspect, the disclosed extracellular vesicles can encapsulate a disclosed polypeptide covalently or non-covalently attached to one or more of a polypeptide, a glycopeptide, a polysaccharide, a glycolipid, a lipid, or a nucleic acid polymer, or a combination thereof. In an aspect, the disclosed extracellular vesicles can encapsulate one or more therapeutic agents. In an aspect, the disclosed extracellular vesicles can encapsulate a disclosed polypeptide covalently or non-covalently attached to one or more disclosed therapeutic agents. In an aspect, the disclosed extracellular vesicles can encapsulate one or more disclosed therapeutic agents.

In an aspect of a disclosed method, secreted extracellular vesicles and/or AAV particles can contact one or more other cells. In an aspect of a disclosed method, an encoded polypeptide can localize to extracellular vesicles and/or AAV particles secreted from a cell. In an aspect of a disclosed method, a fusion product can localize to extracellular vesicles and/or AAV particles secreted from the cell. In an aspect of a disclosed method. AAV particles can localize to extracellular vesicles secreted from a cell.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ TID NO:09, SEQ ID NO:10, SEQ ID NO:11, SEQ ID NO:12, SEQ ID NO:13, SEQ ID NO:14, SEQ ID NO:15, or a fragment thereof. Table 1 shows the serotype for each of SEQ ID NOS:01-15. In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or to a combination thereof. In an aspect, a disclosed polypeptide (e.g., MAAP) can be non-covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA. mRNA, non-coding RNA (ncRNA), an antisense molecule, miRNA, a morpholino, a peptide-nucleic acid (PNA), or an analog or conjugate thereof: In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90%, or at least 95% identity to the sequence set forth in SEQ ID NO:34 or a fragment thereof.

In an aspect, a subject can be a human or a non-human primate.

In an aspect of a disclosed method, expressing the encoded fusion product can comprise transient expression or stable expression. In an aspect, a disclosed method can comprise encapsulating the encoded fusion product in one or more extracellular vesicles and/or AAV particles.

In an aspect, a disclosed vector can be a viral vector or a non-viral vector. A disclosed vector can comprise one or more regulatory elements. In an aspect, a disclosed non-viral vector can be a polymer based vector, a peptide based vector, a lipid nanoparticle, a solid lipid nanoparticle, or a cationic lipid based vector. In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T-V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect of a disclosed method, a vector can be administered via intravenous, intraarterial, intramuscular, intraperitoneal, subcutaneous, intrathecal, intraventricular, or in utero administration. In an aspect, a vector can be administered via LNP administration. In an aspect, a vector can be delivered to the subject's liver, heart, skeletal muscle, smooth muscle, CNS, PNS, or a combination thereof.

### 3. Methods of Improving Viral Particle Formation and Egress

Disclosed herein is a method of improving viral particle egress from a cell comprising delivering to a cell an isolated nucleic acid molecule comprising a nucleic acid sequence encoding (i) a polypeptide for promoting the formation of extracellular vesicles and/or AAV particles in cell or (ii) a polypeptide associated with extracellular vesicles and/or AAV particles secreted from a cell; expressing the encoded polypeptide; and encapsulating viral particles in one or more extracellular vesicles and/or AAV particles.

Disclosed herein is a method of altering or modifying the dynamics of extracellular vesicle and/or AAV particle formation and/or secretion from a cell, comprising delivering to a cell an isolated nucleic acid molecule comprising a nucleic acid sequence encoding (i) a polypeptide for promoting the formation of extracellular vesicles and/or AAV particles in cell or (ii) a polypeptide associated with extracellular vesicles and/or AAV particles secreted from a cell; and expressing the encoded polypeptide.

Disclosed herein is a method of altering or modifying the dynamics of extracellular vesicle and/or AAV particle formation and/or secretion from a cell, comprising delivering to a cell an isolated nucleic acid molecule comprising a nucleic acid sequence encoding a fusion product, wherein the fusion product encodes at least a polypeptide for promoting the formation of extracellular vesicles and/or AAV particles in cell or (ii) a polypeptide associated with extracellular vesicles and/or AAV particles secreted from a cell; and expressing the encoded polypeptide.

In an aspect, altering or modifying the dynamics of extracellular vesicle and/or AAV particle secretion can comprise increasing the rate of particle secretion, increasing the rate of particle formation, or both. In an aspect, altering or modifying the dynamics of extracellular vesicle and/or AAV particle secretion can comprise decreasing the rate of particle secretion, decreasing the rate of particle formation, or both. In an aspect, altering or modifying the dynamics of extracellular vesicle and/or AAV particle secretion can comprise affecting one or more aspects of the formation and/or secretion pathway.

In an aspect, a disclosed encoded polypeptide can alter or modify the dynamics of extracellular vesicle and/or AAV particle secretion. In an aspect, altering or modifying the dynamics of extracellular vesicle and/or AAV particle secretion can comprise increasing the rate of particle secretion, increasing the rate of particle formation, or both. In an aspect, altering or modifying the dynamics of extracellular vesicle and/or AAV particle secretion can comprise decreasing the rate of particle secretion, decreasing the rate of particle formation, or both. In an aspect, altering or modifying the dynamics of extracellular vesicle and/or AAV particle secretion can comprise affecting one or more aspects of the extracellular vesicle and/or AAV particle formation and/or secretion pathway.

In an aspect of a disclosed method, secreted extracellular vesicles and/or AAV particles can comprise one or mom targeting moieties. Targeting moieties are known to the art.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. Table 1 shows the serotype for each of SEQ ID NOS:01-15. In an aspect of a disclosed method, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15.

In an aspect of a disclosed method, a disclosed nucleic acid sequence can have the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof. In an aspect of a disclosed method, a disclosed nucleic acid sequence can have a sequence having at least 30%, at least 40%, at least 50%, at least 60%, at least 70%, at least 80%, or at least 90% identity to the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

In an aspect, a disclosed nucleic acid for a MAAP can be a derivative or an analog of the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30.

In an aspect, a disclosed nucleic acid for a MAAP can comprise the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, and SEQ ID NO:30, wherein the sequence can comprise one or more mutations. In an aspect, the one or more mutations can affect the functionality of the encoded MAAP.

In an aspect, delivering a disclosed isolated nucleic acid molecule can comprise using a vector. In an aspect, a disclosed vector can comprise one or more regulatory elements. In an aspect, a disclosed vector can be a viral vector or a non-viral vector. In an aspect, a disclosed non-viral vector can be a polymer based vector, a peptide based vector, a lipid nanoparticle, a solid lipid nanoparticle, or a cationic lipid based vector.

In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh0, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829, AAV2 Y/F, AAV2 T/V, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, expressing the encoded polypeptide can comprise transient expression or stable expression.

In an aspect, the disclosed extracellular vesicles can encapsulate AAV particles. In an aspect, the disclosed extracellular vesicles and/or AAV particles can encapsulate a disclosed polypeptide. In an aspect, the disclosed extracellular vesicles and/or AAV particles can encapsulate a disclosed polypeptide covalently or non-covalently attached to one or more of a polypeptide, a glycopeptide, a polysaccharide, a glycolipid, a lipid, or a nucleic acid polymer, or a combination thereof. In an aspect, the disclosed extracellular vesicles and/or AAV particles can encapsulate one or more therapeutic agents. In an aspect, the disclosed extracellular vesicles and/or AAV particles can encapsulate a disclosed polypeptide covalently or non-covalently attached to one or more disclosed therapeutic agents. In an aspect, the disclosed extracellular vesicles and/or AAV particles can encapsulate one or more disclosed therapeutic agents. In an aspect of a disclosed method, an encoded polypeptide can localize to extracellular vesicles and/or AAV particles secreted from a cell. In an aspect of a disclosed method, AAV particles can localize to extracellular vesicles secreted from a cell. In an aspect of a disclosed method, secreted extracellular vesicles and/or AAV particles can contact one or more other cells.

In an aspect, a disclosed cell can be a mammalian cell or a non-mammalian cell or a eukaryotic cell or a prokaryotic cell. In an aspect, a disclosed cell can be a human cell. In an aspect, a disclosed cell can be in a subject. In an aspect, a subject can be a human or a non-human primate. In an aspect, a disclosed cell can be in culture. In an aspect, a disclosed method can comprise harvesting the secreted extracellular vesicles and/or AAV particles from conditioned media of the culture.

4. Methods of Loading Extracellular Vesicles and/or AAV Particles with a Cargo

Disclosed herein is a method of loading extracellular vesicles and/or AAV particles with a cargo comprising delivering to a cell an isolated nucleic acid molecule comprising a nucleic acid sequence encoding a polypeptide for promoting the formation of extracellular vesicles and/or AAV particles; and expressing an encoded polypeptide, wherein the encoded polypeptide is directed to extracellular vesicles and/or AAV particles.

Disclosed herein is a method of loading extracellular vesicles and/or AAV particles with a cargo comprising delivering to a cell an isolated nucleic acid molecule comprising a nucleic acid sequence encoding a polypeptide associated with extracellular vesicles and/or AAV particles; and expressing an encoded polypeptide, wherein the encoded polypeptide is directed to an extracellular vesicle and/or AAV particle.

Disclosed herein is a method of loading extracellular vesicles and/or AAV particles with a cargo comprising delivering to a cell an isolated nucleic acid molecule comprising a nucleic acid sequence encoding a fusion product, expressing an encoded fusion product comprising (i) a polypeptide promoting the formation of extracellular vesicles and/or AAV particles in cell and (ii) cargo; wherein the fusion product is directed to an extracellular vesicle and/or AAV particle. Disclosed herein is a method of loading extracellular vesicles and/or AAV particles with a cargo comprising delivering to a cell an isolated nucleic acid molecule comprising a nucleic acid sequence encoding a fusion product, expressing an encoded fusion product comprising (i) a polypeptide associated with extracellular vesicles and/or AAV particles secreted from a cell and (ii) cargo; wherein the fusion product is directed to an extracellular vesicle and/or AAV particle.

In an aspect, a disclosed encoded polypeptide can alter or modify the dynamics of extracellular vesicle and/or AAV particle secretion. In an aspect, altering or modifying the dynamics of extracellular vesicle and/or AAV particle secretion can comprise increasing the rate of vesicle and/or particle secretion, increasing the rate of vesicle and/or particle formation, or both. In an aspect, altering or modifying the dynamics of extracellular vesicle and/or AAV particle secretion can comprise decreasing the rate of vesicle and/or particle secretion, decreasing the rate of vesicle and/or particle formation, or both. In an aspect, altering or modifying the dynamics of extracellular vesicle and/or AAV particle secretion can comprise affecting one or more aspects of the formation and/or secretion pathways.

In an aspect, a disclosed encoded polypeptide can be a membrane-associated accessory protein (MAAP) or a fragment thereof. MAAP can comprise an N-terminal hydrophobic domain linked to cationic, amphipathic C-terminal domain. In an aspect, a disclosed MAAP can have the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof. Table 1 shows the serotype for each of SEQ ID NOS:01-15. In an aspect, a disclosed MAAP can have a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO: 11, SEQ ID NO:12, SEQ ID NO:13, SEQ ID NO:14, SEQ ID NO:15, or a fragment thereof. For example, in an aspect, a disclosed encoded polypeptide can have a sequence having at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof. In an aspect, a disclosed encoded polypeptide can comprise the sequence set forth in SEQ ID N0:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

In an aspect, a disclosed MAAP can be a derivative or an analog of the MAAP having a sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ TID NO:15.

In an aspect, a disclosed polypeptide (e.g., MAAP) can be covalently attached or non-covalently attached to one or more of a polypeptide, a glycopeptide, a polysaccharide, a glycolipid, a lipid, or a nucleic acid polymer, or to a combination thereof.

In an aspect, a disclosed polypeptide (e.g., MAAP) can be non-covalently attached or non-covalently attached to one or more therapeutic agents.

In an aspect, a disclosed isolated nucleic acid molecule can comprise the sequence for at least one of polypeptide, a glycopeptide, a polysaccharide, a glycolipid, a lipid, or a nucleic acid polymer, or a combination thereof. In an aspect, a disclosed isolated nucleic acid molecule can comprise the sequence for at least one therapeutic agent. In an aspect, a disclosed therapeutic agent can be an oligonucleotide therapeutic agent. In an aspect, a disclosed oligonucleotide therapeutic agent can be a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof. In an aspect, a disclosed therapeutic agent can be an ASO or an RNAi. In an aspect, a disclosed nucleic acid-based molecule can comprise one or more modifications at any position applicable. In an aspect, a disclosed therapeutic agent can comprise a CRISPR-based endonuclease.

In an aspect, a disclosed endonuclease can be Cas9. In an aspect, a disclosed Cas9 can be from Staphylococcus aureus or Streptococcus pyogenes. In an aspect, a disclosed Cas9 can have the sequence set forth in SEQ ID NO:33 or a fragment thereof. In an aspect, a disclosed Cas9 can have a sequence having at least 75%, at least 80%, at least 85%, at least 90%, or at least 95% identity to the sequence set forth in SEQ ID NO:33 or a fragment thereof. In an aspect, a nucleic acid sequence for Cas9 can comprise the sequence set forth in SEQ ID NO:34 or a fragment thereof. In an aspect, a disclosed nucleic acid sequence for Cas9 can comprise a sequence having at least 80%, at least 85%, at least 90.%, or at least 95% identity to the sequence set forth in SEQ ID NO:34 or a fragment thereof. In an aspect, a disclosed nucleic acid-based cargo molecule can comprise one or more modifications at any position applicable.

In an aspect, a disclosed method can comprise secreting extracellular vesicles and/or AAV particles from the cell. In an aspect, a disclosed method can comprise contacting the secreted extracellular vesicles and/or AAV particles with one or more cells. In an aspect, secreted extracellular vesicles and/or AAV particles can comprise one or more targeting moieties.

Targeting moieties are known to the art. In an aspect of a disclosed method, secreted extracellular vesicles and/or AAV particles can contact one or more other cells. In an aspect, a disclosed method can comprise harvesting the secreted extracellular vesicles and/or AAV particles from conditioned media of the culture.

In an aspect, delivering a disclosed isolated nucleic acid molecule can comprise using a vector. In an aspect, a disclosed vector can comprise one or more regulatory elements. In an aspect, a disclosed vector can be a viral vector or a non-viral vector. In an aspect, a disclosed viral vector can be an adenovirus vector, an adeno-associated virus (AAV) vector, a herpes simplex virus vector, a retrovirus vector, a lentivirus vector, and alphavirus vector, a flavivirus vector, a rhabdovirus vector, a measles virus vector, a Newcastle disease viral vector, a poxvirus vector, or a picornavirus vector.

In an aspect, a disclosed viral vector can be an AAV vector. In an aspect, a disclosed AAV vector can be AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, or AAVcy.7. In an aspect, a disclosed AAV vector can be bovine AAV, caprine AAV, canine AAV, equine AAV, ovine AAV, avian AAV, primate AAV, or non-primate AAV. In an aspect, a disclosed AAV vector can be AAV-DJ, AAV-HAE1, AAV-HAE2, AAVM41, AAV-1829. AAV2 Y/F, AAV2 TV, AAV2i8, AAV2.5, AAV9.45, AAV9.61, AAV-B1, AAV-AS, AAV9.45A-String (e.g., AAV9.45-AS), AAV9.45Angiopep, AAV9.47-Angiopep, AAV9.47-AS, AAV-PHP.B, AAV-PHP.eB, AAV-PHP.S, AAV-F, AAVcc.47, or AAVcc.81.

In an aspect, a disclosed non-viral vector can be a polymer based vector, a peptide based vector, a lipid nanoparticle, a solid lipid nanoparticle, or a cationic lipid based vector.

In an aspect of a disclosed method, a vector can be administered via intravenous, intraarterial, intramuscular, intraperitoneal, subcutaneous, intrathecal, intraventricular, or in utero.

In an aspect, a disclosed cell can be a mammalian cell or a non-mammalian cell or a eukaryotic cell or a prokaryotic cell. In an aspect, a disclosed cell can be a human cell. In an aspect, a disclosed cell can be in a subject. In an aspect, a subject can be a human or a non-human primate. In an aspect, a disclosed cell can be in culture.

### EXAMPLES

AAVs belong to the genus dependoparvovirus in the family Parvovirinae. The model species is dependoparvovirus A, of which the prototype strain is AAV2. AAVs encode a replicase protein and a capsid protein, of which 3 isoforms are made—VP1, VP2, and VP3. AAV2 encodes 2 additional proteins in reading frames overlapping the capsid—AAP (Assembly-Activating Protein) and MAAP (Membrane-Associated Accessory Protein, which is the subject of the methods and compositions disclosed herein.

MAAP is translated from a non-canonical start codon—CTG. It is thought that overlapping gene arrangements, such as VP1/MAAP, originate via a process called “overprinting”. Here, one or more mutations in an ancestral reading frame enable the expression of a second reading frame while simultaneously preserving the expression of the first reading frame. Consequently, each pair of overlapping reading frames contains one ancestral frame and one originated de novo (as compared to the classical means of gene origination by duplication or horizontal gene transfer).

Proteins originated de novo by overprinting generally have a highly biased composition tend to be structurally disordered. These proteins also tend evolve faster than the ancestral reading frame. These proteins often play an important role in viral pathogenicity, for instance by neutralizing the host interferon response or by inducing apoptosis in host cells. Those characterized so far have previously unknown mechanisms of action, and the minority that are not disordered have previously unknown 3D structural folds.

Here, the identification of MAAP as a unique virally encoded protein having (i) an amphipathic, cationic membrane binding domain. (ii) a short, disordered N-terminus that can bind to other molecules and proteins, and (iii) a linking domain. Moreover, the data provided herein confirm that the membrane-associated accessory protein (MAAP), which is expressed from a (+1) frameshifted open reading frame (ORF) in the N-terminal region of the AAV capsid (Cap) gene, is an AAV cellular egress factor.

### A. Materials and Methods

### 1. Plasmid Constructs

MAAP DNA sequences from AAV1, AAV2. AAV5, AAV8, and AAV9 were synthesized and cloned into pcDNA3.1(+)-C-HA and pcDNA3.1(+)-C-eGFP expression vectors using HindIII and XbaI sites for MAAP 1, 2, 8, 9 and EcoRV sites for MAAP5 (Genscript). All MAAP expression constructs were synthesized and cloned with an ATG start codon. The AAV8-Rep/Cap-VP* plasmid is a 2rep/8cap plasmid with the start codons of VP1, VP2, and VP3 and AAP mutated by site directed mutagenesis to prevent expression. The AAV8-Rep/Cap-MVP* additionally had a mutated MAAP start codon to prevent MAAP expression.

### 2. Bioinformatics Analysis and Structural Models

The amino acid sequences of 15 AAV serotypes were retrieved from GenBank. MAAP start and stop sites were defined as previously described (Ogden et al. 2009). Protein sequences were aligned using the ClustalW multiple-alignment tool (Thompson J D, et al. 1994 Nucleic Acids Res. 22(22):4673-4680) and generated using Unipro UGENE software (Okonechnikov K, et al. 2012 Bioinformatics. 28(8):1166-1167). MAAP amino acid sequences from multiple AAV isolates were aligned using ClustalW, and phylogenetic trees were generated using the MEGAv7.0.21 software package (Kumar S, et al. 2016 Mol Biol Evol. 33(7):1870-1874). The phylogeny was produced using the neighbor-joining algorithm, and amino acid distances were calculated using a Poisson correction (Saitou N, et al. 1987 Mol Biol Evol. 4(4):406-425). Statistical testing was done by bootstrapping with 1,000 replicates to test the confidence of the phylogenetic analysis and to generate the original tree (Felsenstein J. 1985 Evolution. 39(4):783-791). The percentage of replicate trees in which associated taxa clustered together in the bootstrap test is displayed next to the branches. Secondary structural elements were predicted using the J Pred tool (Cole C, et al. 2008 Nucleic Acids Res. 36:W197-201). To predict membrane-binding, amphipathic α-helices, we used Amphipaseek (parameters: high specificity/low sensitivity) (Sapay N, et al. 2006 BMC Bioinformatics. 7:255). MAAP structural models were generated using the Protein Homology/analogY Recognition Engine v. 2.0 (Phyre2) intensive modeling construction (Kelley L A, et al. 2015 Nat Protoc. 10(6): 845-858). Homology structural models were generated from crystal structures of multiple templates. Secondary structural depictions of these models were visualized using the PyMOL Molecular Graphics System (Schrödinger, https://www.pymol.org/2/).

### 3. Cellular Assays, Immunoprecipitations, and Western Blotting

For protein expression analysis, HEK293 cells seeded overnight in 6-well plates at a density of 3-10⁵ cells per plate were transfected with a total of 2 μg DNA as indicated. HEK293 cell pellets overexpressing MAAP-HA or MAAP-GFP were recovered 72 hour post-transfection. Pellets were lysed in RIPA buffer with 1× Halt Protease Inhibitor (ThermoFisher) for 45 minutes at 4° C. Lysates were spun at max speed for 10 minutes at 4° C. to remove cellular debris. 1×LDS sample buffer with 10 mM DTT were added to cleared lysates and boiled for 2 minutes. Samples of cleared lysate were ran on Mini-Protean TGX 4-15% gels (Biorad), transferred onto PVDF with the Trans-Blot Turbo system (BioRad), and blocked in 5% milk/1×TBST. Blots were probed with a mouse monoclonal anti-GFP antibody (1:1000 dilution, SC9996; Santa Cruz Biotechnology), a rabbit polyclonal anti-HA SG77 antibody (1:1000 dilution, 71-5500; ThermoFisher Scientific), a mouse monoclonal B1 hybridoma supernatant (1:50, 03-65158; ARP), or a mouse monoclonal anti-beta Actin (1:1000 dilution, 8226; Abcam) as the primary antibody. Following three 1×TBST washes, samples were incubated with secondary antibodies conjugated to HRP at 1:20,000 in 5% milk/1×TBST for 1 hour (i.e., goat anti-mouse-HRP (#32430 from ThermoFisher Scientific) and goat anti-rabbit-HRP (#111-035-003 from Jackson ImmunoResearch)). Blots were developed using SuperSignal West Femto substrate (ThermoFisherScientific/Life Technologies) according to manufacturer instructions.

For immunoprecipitation studies, HEK293 cells were transfected with pXR9 and MAAP9-pcDNA3.1(+)-C-HA for 72 hours, then washed with 1×PBS, and harvested in NP-40 with 1× Halt Protease Inhibitor (ThermoFisher) for 1 hour at 4° C. Lysates were spun at max speed for 20 minutes at 4° C. to remove cellular debris. Then, 10 μL (2.5 μg) of anti-HA SG77 antibody were added to 500 μL cleared lysate and incubated at 4° C. for 3 hours with nutation. Then, 40 μL of pre-washed Protein G magnetic beads were added to pre-cleared lysate with antibody and carried out immunoprecipitations overnight on a nutator at 4° C. Bound protein was eluted in 10 mM DTT and 1×LDS for 5 min at 95° C. Samples were then analyzed via SDS-PAGE (NuPAGE 4-12% Bis-Tris Gel) and transferred onto nitrocellulose membrane (ThermoScientific). Following blocking in 5% milk/1×TBST, samples were incubated with primary antibodies to either capsid (mouse monoclonal B1 hybridoma supernatant. 1:250 dilution, #65158 from Progen), actin (mouse monoclonal anti-beta Actin, 1:1000 dilution, #8226 from Abcam), or MAAP (mouse monoclonal anti-HA HA.C5 antibody, 1:1000 dilution, #MA5-27543 from ThermoFisher Scientific) overnight in 5% milk/1×TBST. Following three 1×TBST washes, samples were incubated with secondary anti-mouse antibody conjugated to HRP (Goat anti-mouse-HRP, #32430 from ThermoFisher Scientific) at 1:20,000 in 5% milk/1×TBST for 1 hour. The signal was the visualized via SuperSignal West Femto Maximum Sensitivity substrate (ThermoScientific) according to manufacturer instructions.

### 4. AAV Vector Production, Purification, and Quantification

HEK293 (human embryonic kidney cells obtained from the University of North Carolina Vector Core) were maintained in Dulbecco's Modified Eagle's Medium (DMEM) supplemented with 10% fetal bovine serum (FBS). 100 U/mL penicillin, 100 μg/mL streptomycin. Cells were maintained in 5% CO₂ at 37° C. Recombinant AAV vectors were produced by transfecting HEK293 cells at ˜75% confluence with polyethylenimine (PEI) using a triple plasmid transfection protocol with the AAV Rep-Cap plasmid, Adenoviral helper plasmid (pXX680), and single-stranded genomes encoding firefly luciferase driven by the chicken beta-actin promoter (ssCBA-Luc) or self-complementary green fluorescence protein (GFP) driven by a hybrid chicken beta-actin promoter (scCBh-GFP), flanked by AAV2 inverted terminal repeat (ITR) sequences. Viral vectors were harvested from media and purified via iodixanol density gradient ultracentrifugation followed by phosphate buffered saline (PBS) buffer exchange. Titers of purified virus preparations were determined by quantitative PCR using a Roche Lightcycler 480 (Roche Applied Sciences, Pleasanton, CA) with primers amplifying the AAV2 ITR regions. The forward primer is 5′-AACATGCTACGCAGAGAGGGAGTGG-3′ (SEQ ID NO:31) and the reverse primer is 5′-CATGAGACAAGGAACCCCTAGTGATGGAG-3′ (SEQ ID NO:32) (IDT Technologies, Ames IA).

### 5. Quantitative PCR Analysis of AAV Genomes

HEK293 cells in six-well plates were transfected using PEI at ˜75% confluence with Adenovirus helper plasmid (1 μg), WT or MAAPΔ AAV-Rep/Cap plasmid (1 μg), ITR-transgene plasmid (500 ng), and AAV8-Rep/Cap-VP* or -MVP* (500 ng). The AAV8-Rep/Cap-VP* plasmid is a 2rep/8cap plasmid with the start codons of VP1, VP2, and VP3 and AAP mutated to prevent expression. The AAV8-Rep/Cap-MVP* additionally has a mutated MAAP start codon.

For 3-day experiments, media and cells were collected 3 days post-transfection. Cells were lysed by vortexing in a mild lysis buffer (10 mM Tris-HCl, 10 mM MgCl₂, 2 mM CaCl₂, 0.5% Triton X-100 supplemented with DNAse, RNAse, and Halt Protease Inhibitor Cocktail) and incubated at 37° C. for 1 hour. Lysates were cleared by centrifuging at 21.000 rcf for two minutes. NaCl to 300 mM was added to AAV2 lysates prior to centrifugation to prevent virus binding of the cell debris. Collected media and cleared lysates were assayed with qPCR for DNAse-resistant viral genomes as described above. For Day 3 and Day 5 experiments, media was collected and replaced on the first indicated day, and cells and media were harvested as described on the last day. For WT-like AAV8 transfections, cells were transfected with Adenovirus helper plasmid (1.9 μg) and WT or MAAPΔ ITR-2rep/8cap-ITR plasmid (0.9 μg), with media and cells collected on days 3 and 5 post-transfection as described above.

### 6. Confocal Fluorescence Microscopy

HEK293 cells were seeded on slide covers in 24-well plates at a density of 5e⁴ cells/well and allowed to adhere overnight. Cells were then co-transfected with Rab7-GFP, Rab11-GFP, MAAP8-pcDNA3.1(+)-C-HA, and MAAP9-pcDNA3.1(+)-C-HA, and were incubated for 48 hours at 37° C. and 5% CO₂. Cells were then fixed with 4% paraformaldehyde for 30 minutes and permeabilized with 0.1% Triton X-100 for 30 minutes. Following 1 hour of blocking with 5% Normal Goat Serum, cells were stained with rabbit polyclonal anti-HA SG77 antibody (1:100 dilution, 71-5500; ThermoFisher Scientific) primary for 1 hour, washed 3× with PBS, and then stained with fluorescent goat anti-rabbit Alexa Fluor 647 (1:400 dilution, #ab150079; Abcam) secondary antibody and washed 1× with PBS. Cells were subject to 5 minutes staining with DAPI, and then mounted in Prolong Diamond (Invitrogen) and imaged using a Zeiss LSM 880 Airyscan confocal microscope. Co-localization analysis was performed by cropping the whole compartment (Rab11 or FIP3), or the whole cell, using Zeiss ZEN software with the Co-localization function. Threshold was automatically determined using the Costes method autothreshold determination. Pearson's correlation coefficient was calculated for the analysis. Statistical analyses were carried out by the nonparametrical Mann-Whitney U test using Prism software (GraphPad).

### 7. Exosome Isolation

Exosomes were isolated from tissue culture media using a commercial kit containing a polyethylene glycol (PEG) solution (ExoQuick-TC ULTRA kit, EQULTRA-20TC-1; System Biosciences) according to the manufacturer's instructions.

### 8. Transmission Electron Microscopy

Isolated exosomes or AAV viral particles (1×10¹⁰ vg) in 1×PBS samples were adsorbed onto 400 mesh, carbon coated grids (Electron Microscopy Sciences) for 2 min, and briefly stained with 1% uranyl acetate (Electron Microscopy Sciences) diluted in 50% ethanol. After drying, grids were imaged with a Philips CM12 electron microscope operated at 80 kV. Images were collected on an AMT camera.

### 9. Luciferase Expression Assays

A total of 1×10⁴ HEK293 cells in 50 μL DMEM+10% PBS+penicillin streptomycin was then added to each well, and the plates were incubated in 5% CO₂ at 37° C. for 24 hours. Cells were then transduced with AAV8-ssCBA-Luc vectors at a dose of 10,000 and 50,000 vg/cell. 48 hours post-transduction, cells were then harvested and lysed with 25 μL of 1× passive lysis buffer (Promega) for 30 minutes at room temperature. Luciferase activity was measured on a Victor 3 multi-label plate reader (PerkinElmer) immediately after the addition of 25 μL of luciferin (Promega).

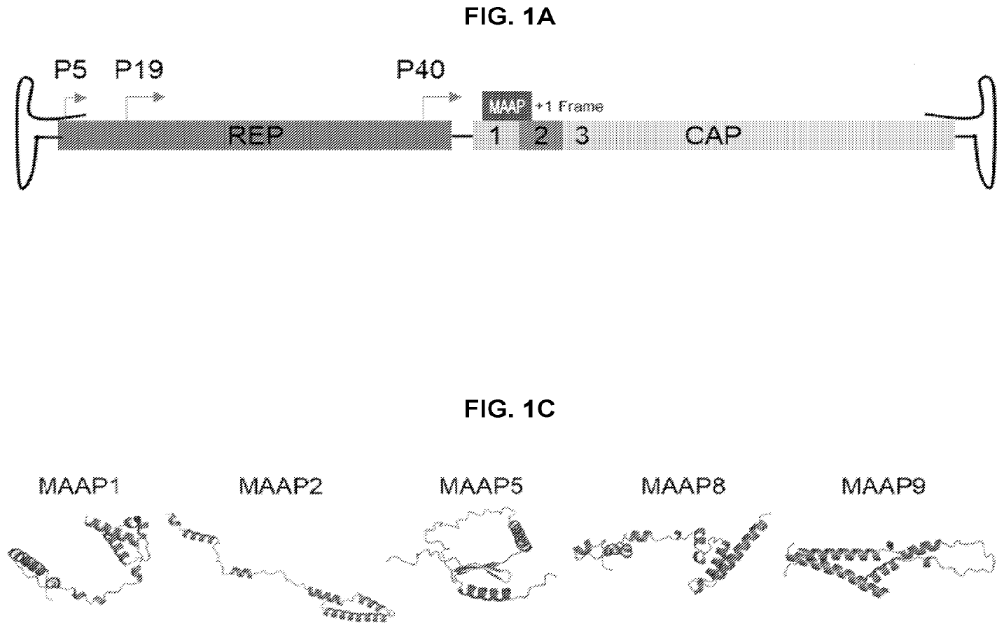

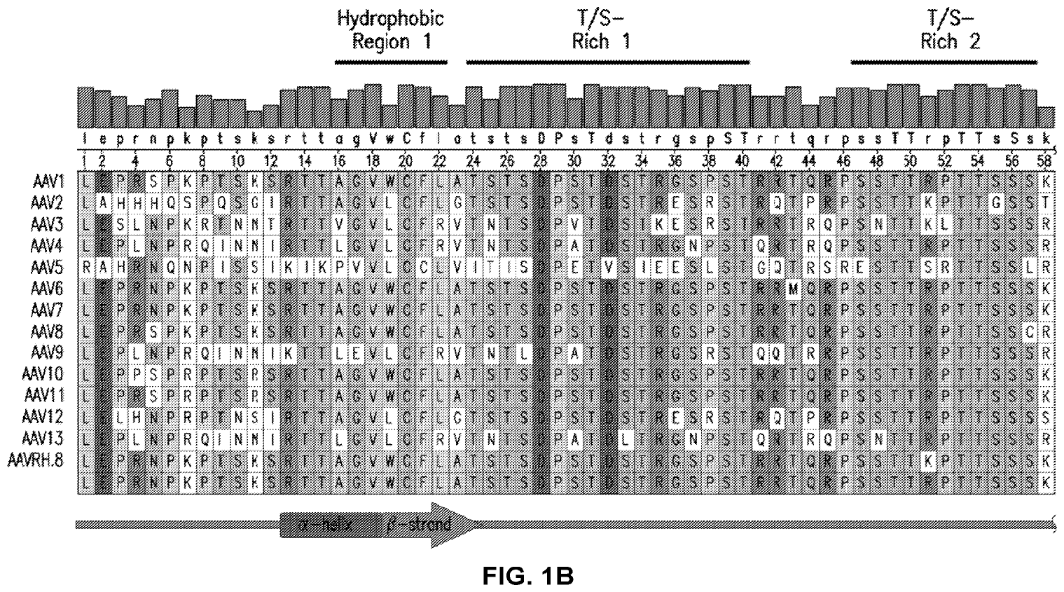

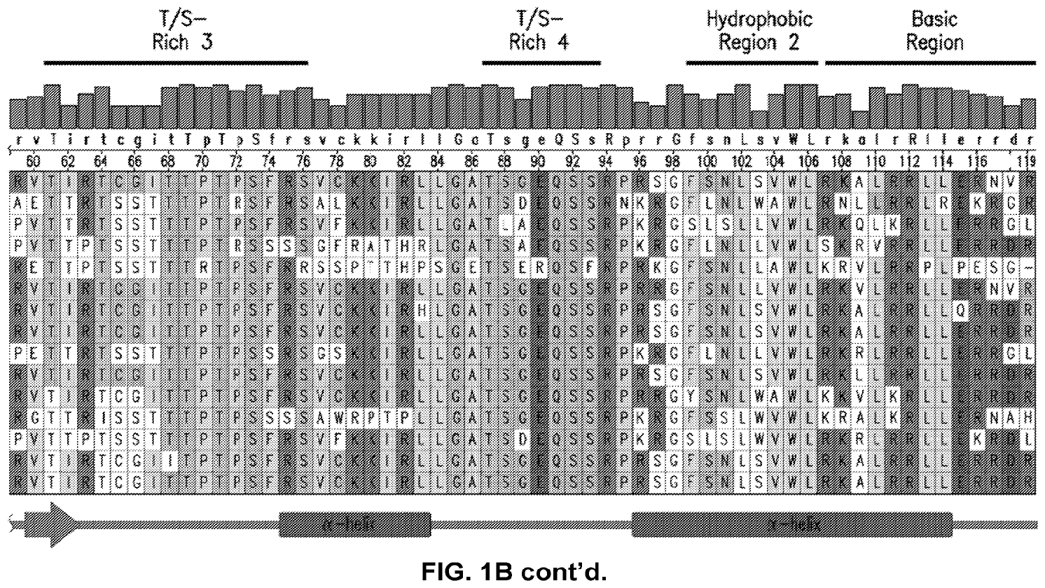

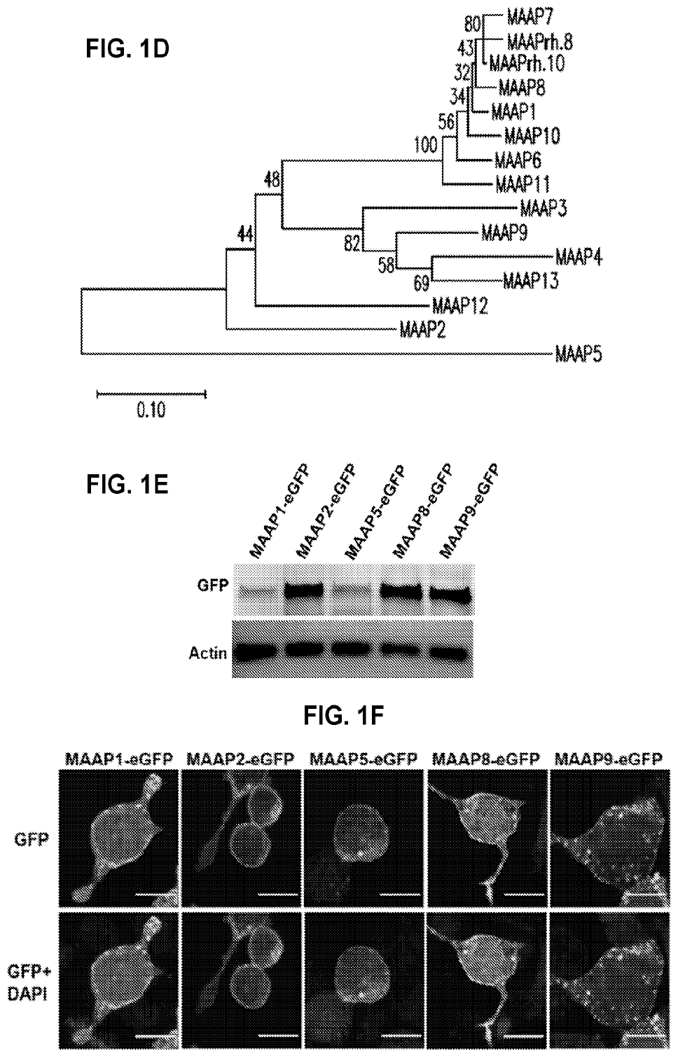

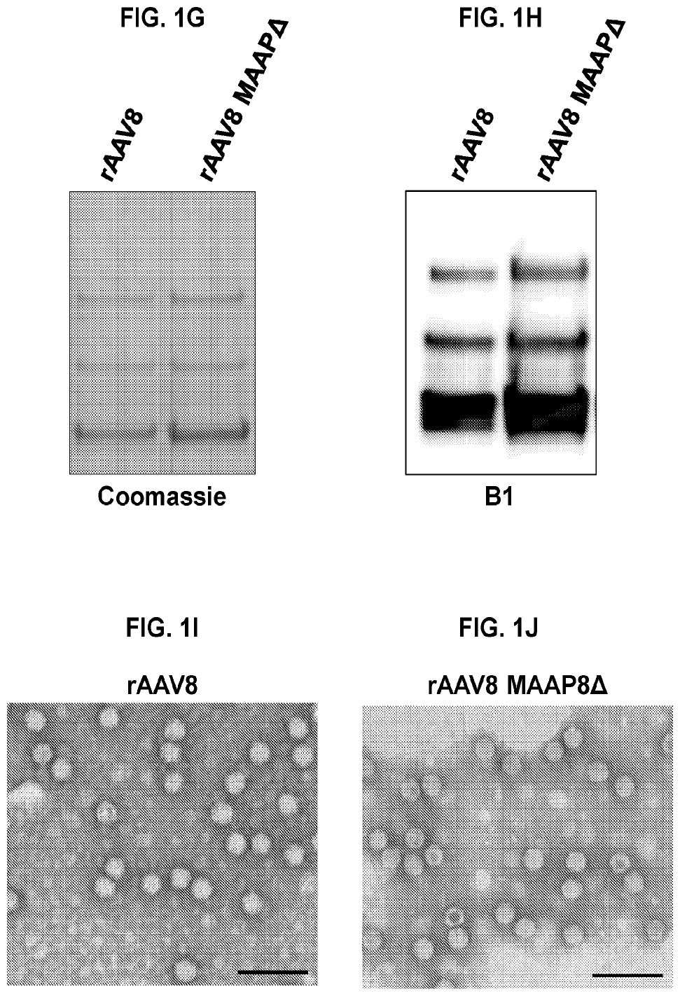

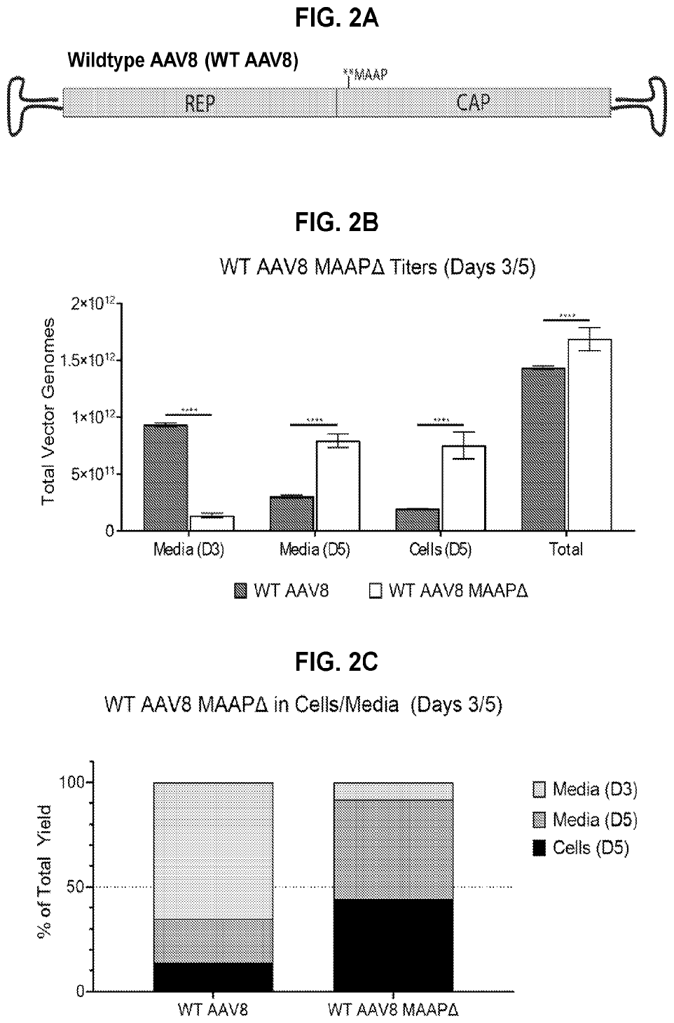

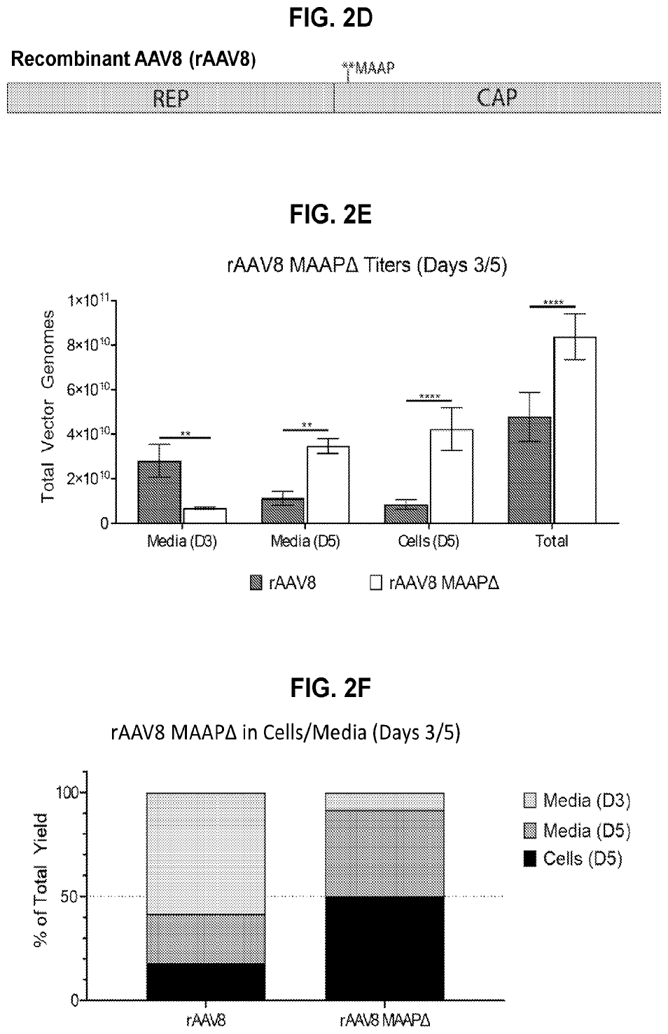

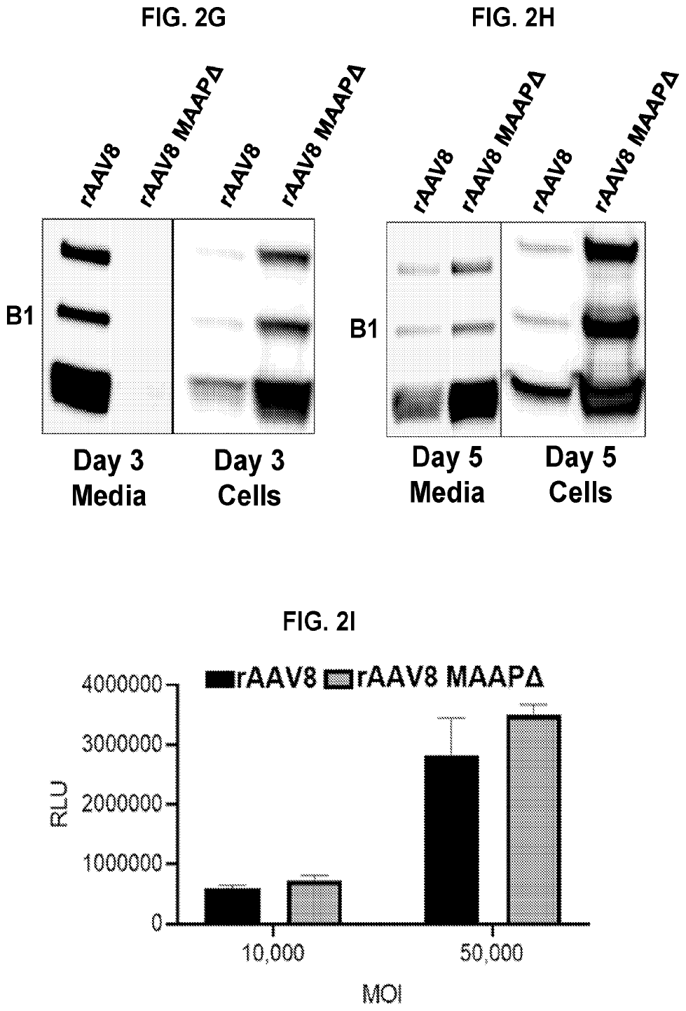

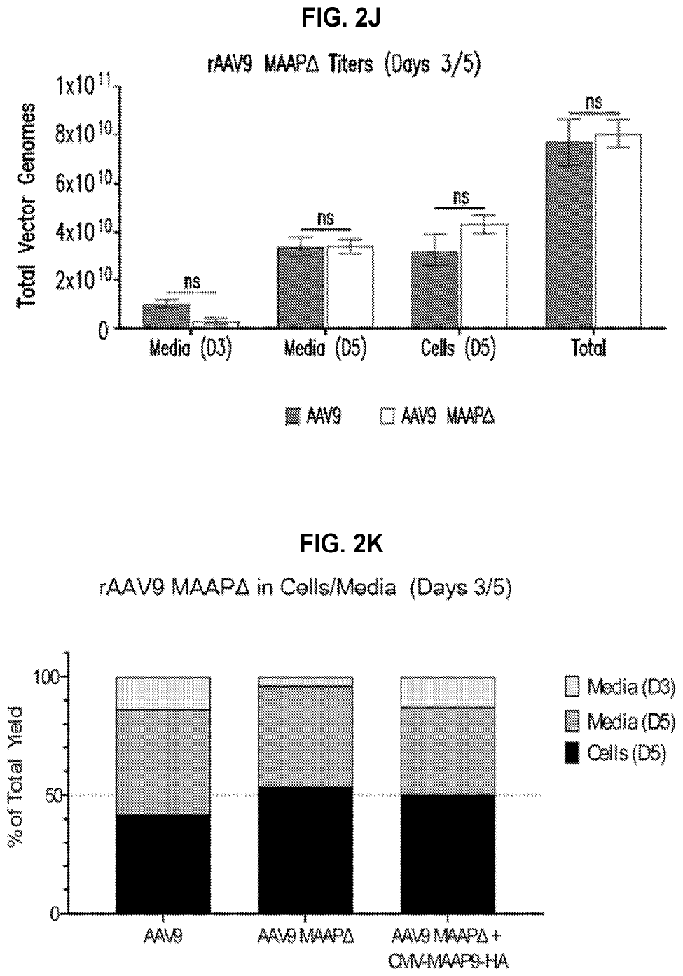

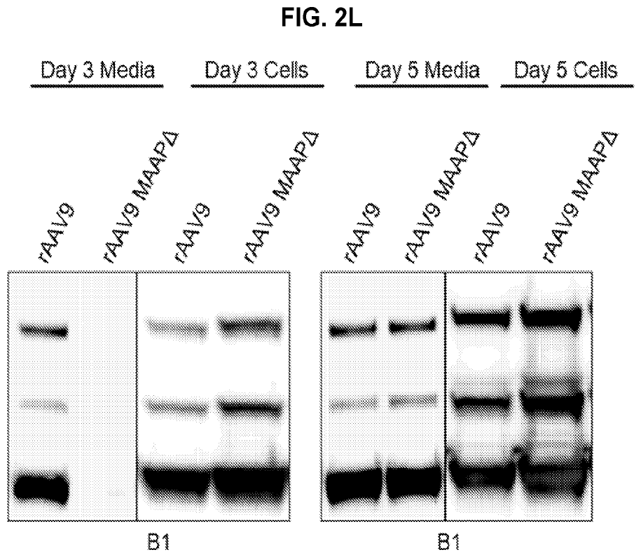

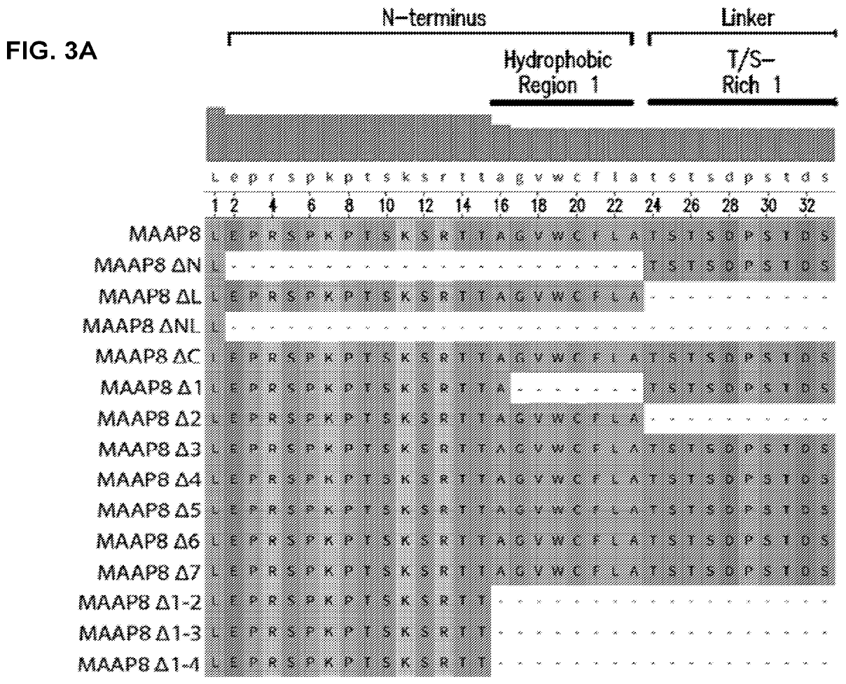

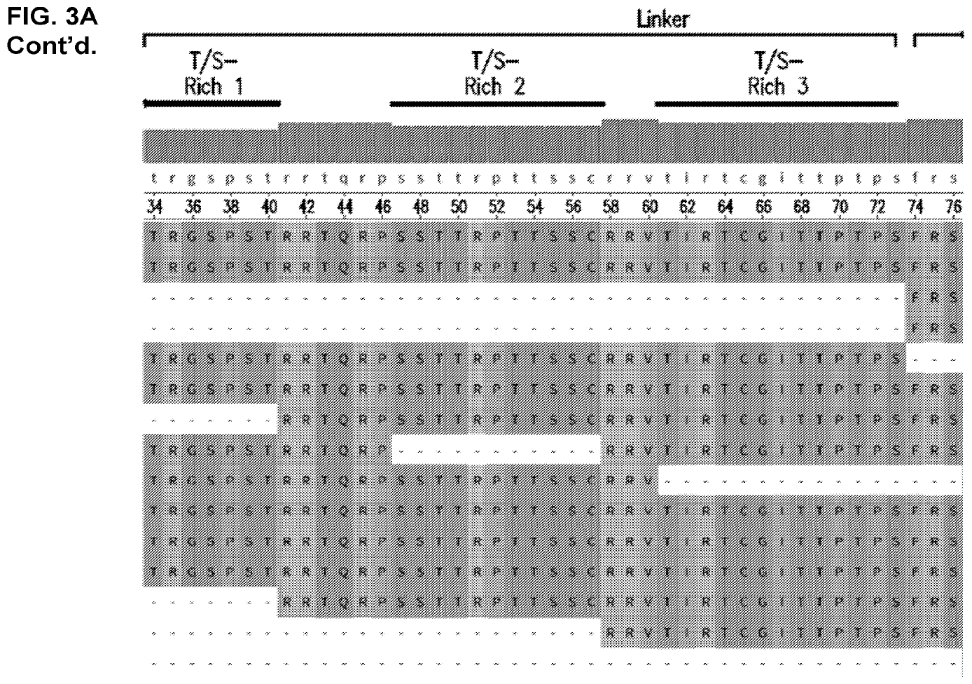

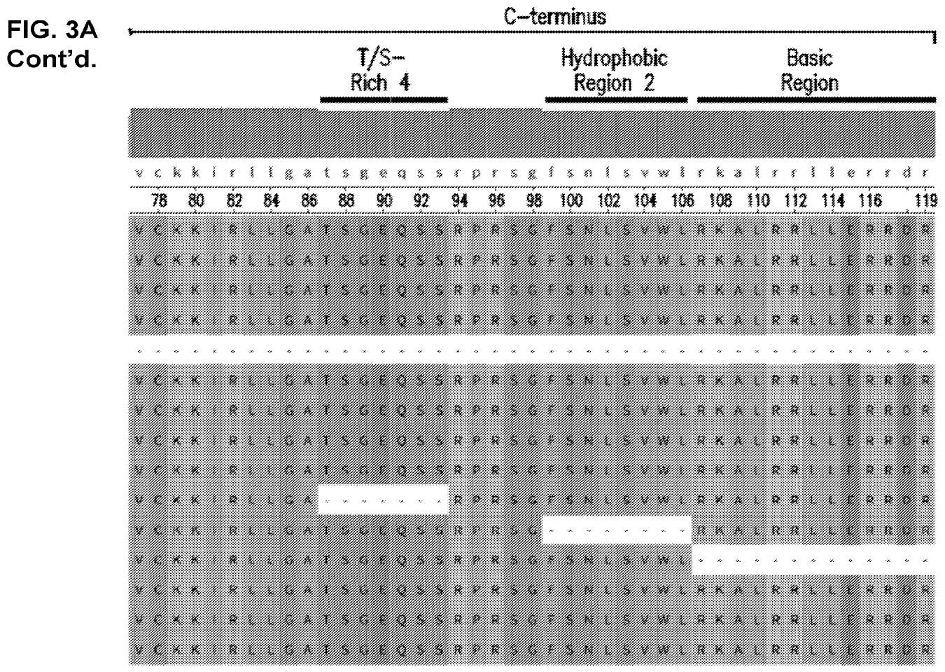

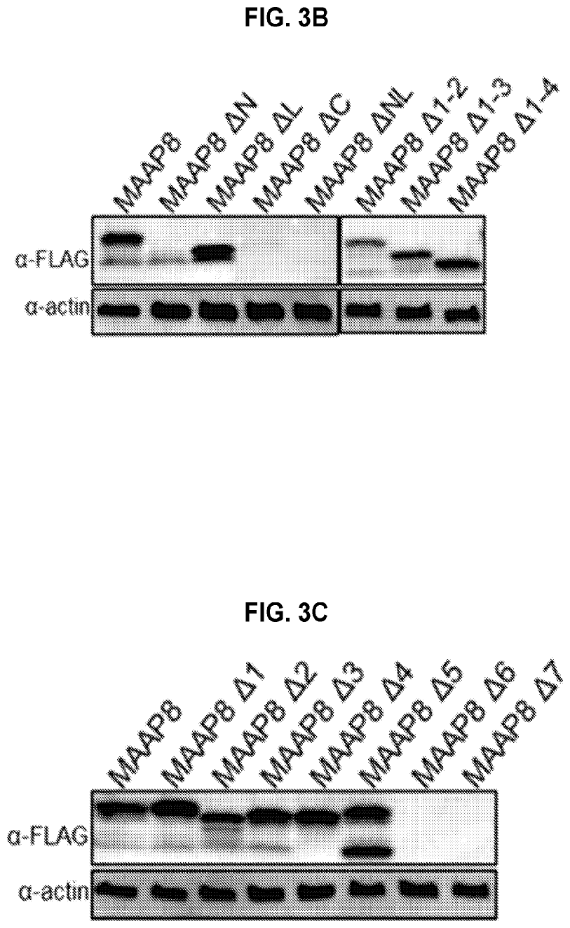

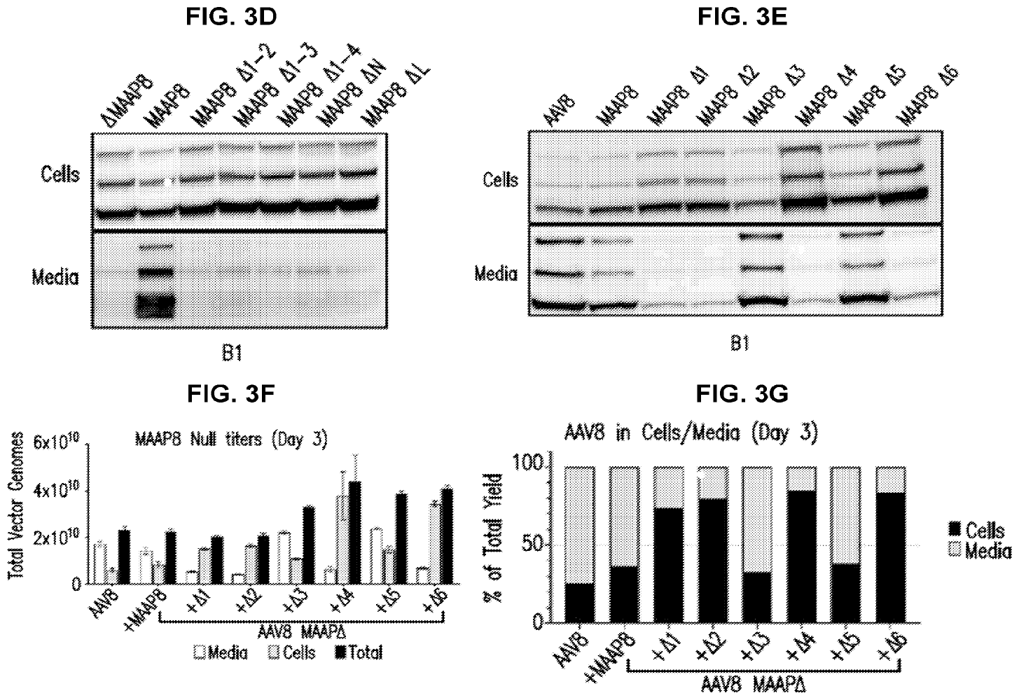

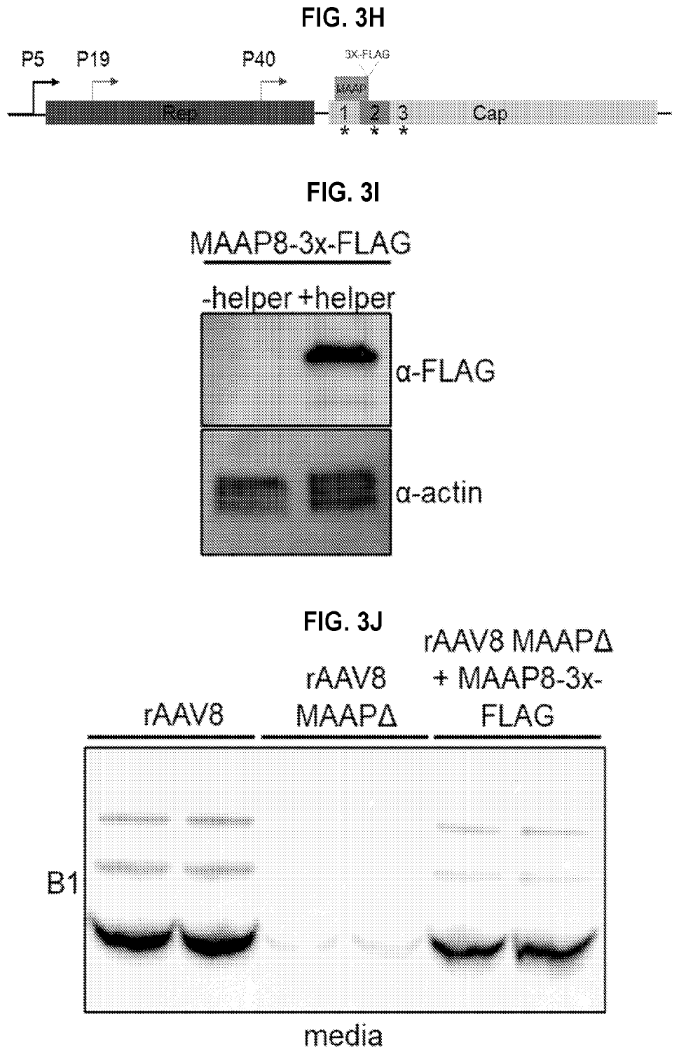

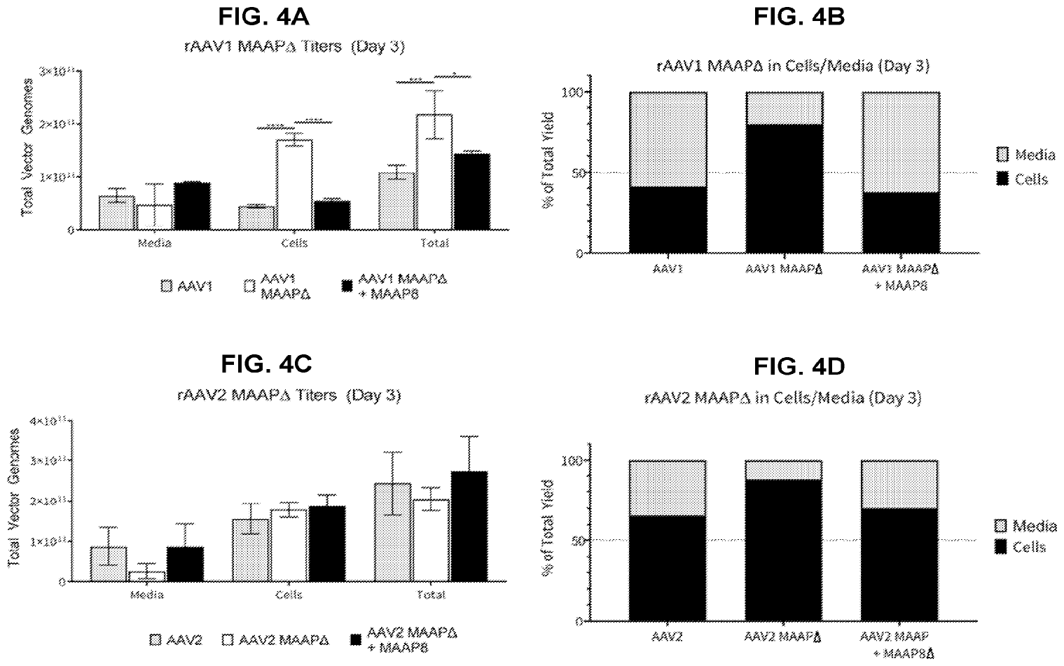

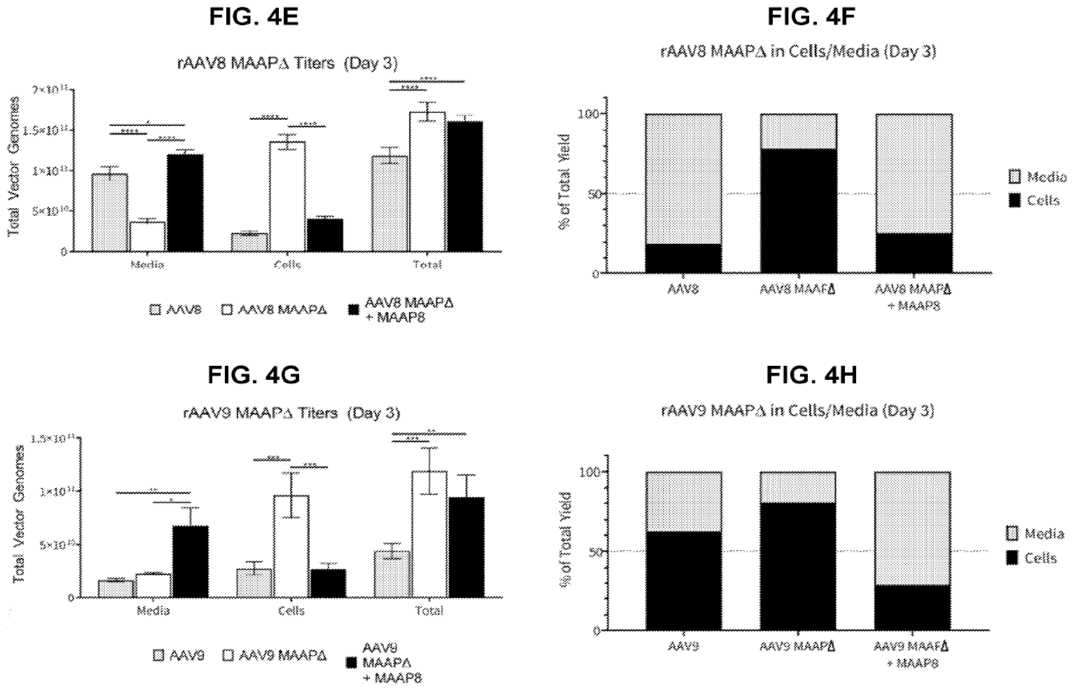

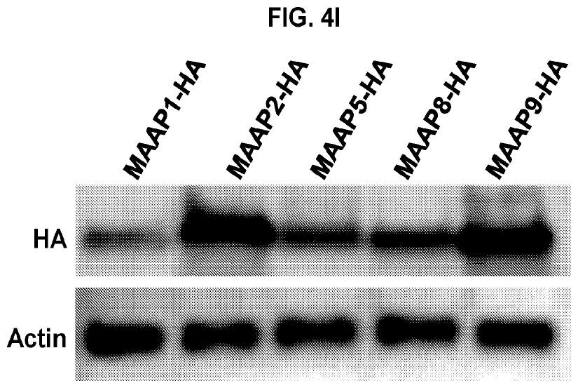

### 10. Statistical Analysis

Where appropriate, data are represented as mean or mean±standard deviation. For data sets with two groups (FIG. 2B, FIG. 2E, and FIG. 1F-1I), comparisons were made between all groups and significance was determined using a two-way ANOVA followed by a Sidak's post-test. For data sets with at least three groups (FIG. 3), comparisons were made between all groups and significance was determined by two-way ANOVA, with Tukey's post-test. For analysis of confocal microscopy data (FIG. 4A and FIG. 4I) significance was determined by a Mann-Whitney rank test. *p<0.05. **p<0.01, ***p<0.001. ****p<0.0001.

### B. Specific Examples

### Example 1

### MAAPS Share Conserved N- and C-Terminal Regions

Recent work has revealed a novel+1 frameshifted open reading frame (ORF) in the VP1 region of the AAV cap gene that mediates expression of the membrane-associated accessory protein (MAAP), which was postulated to limit AAV production through competitive exclusion (Ogden et al. 2019). FIG. 1A, for example, shows a wild-type AAV genome having Rep and Cap genes with MAAP encoded in a +1 reading frame in the VP1 region. Confirming that MAAP is a novel viral-encoded protein of unknown function, a pBLAST search of multiple AAV cap gene derived MAAP sequences on the National Center for Biotechnology Information (NCBI) website did not return any proteins with significant homology (Ogden et al. 2019). Amino acid sequence alignment of MAAPs derived from different AAV serotypes revealed conserved N- and C-terminal regions containing hydrophobic and basic amino acid residues interconnected by a threonine/serine (T/S) rich region (FIG. 1B).

### Example 2

MAAP's Cationic, Amphipathic C-Terminal Domain Associates with Cell Surface and Subcellular Membrane

Three-dimensional (3D) structural modelling on Phyre² predicted a mostly unstructured protein with the following features: (i) a conserved N-terminal hydrophobic motif with both alpha helical and beta strand secondary structure elements; (ii) four T/S rich sequence clusters spanning 7-17 residues in length with last two being separated by a smaller alpha helical interspersed with basic residues, and (iii) a C-terminal domain defined by another hydrophobic alpha helical motif merging into a cluster of arginine/lysine (R/K) residues (FIG. 18 and FIG. 1C). The secondary structure of MAAP is strikingly similar to the assembly activating protein (AAP), which is similarly encoded downstream from a (+1) frameshifted ORF in the cap gene. Importantly, the secondary structure of MAAP is highlighted by an N-terminal hydrophobic domain linked to a cationic, amphipathic C-terminal domain (the putative membrane binding domain—residues 96-114), which strongly associates with the cell surface and subcellular membrane. Thus, these data show that MAAP is a unique virally encoded protein with an amphipathic, cationic membrane anchoring domain.

### Example 3

### Some but not all of the AAV MAAPs are Tightly Clustered

When combined with phylogenetic analysis using the neighbor-joining tree method, MAAPs from AAV1, AAV6, AAV8, AAV10, and AAV I1 were observed to be tightly clustered, while other sequences, in particular, MAAP from AAV5 and AAV9 showed significant divergence from other serotypes (FIG. 1D).

### Example 4

MAAP Associates with Cell Surface Membranes as Well as Other Subcellular Organelles

Plasmids encoding recombinant MAAPs derived from the VP1 sequences of AAV1, AAV2, AAV5, AAV8, and AAV9 and fused to a C-terminal green fluorescent protein (GFP) were transfected into HEK293 cells in vitro to assess their expression (FIG. 1E) and cellular localization. Fluorescence micrographs confirmed the propensity of MAAP to associate with cell surface membranes as well as subcellular organelles, which was evidenced by the punctate patterns throughout the cell (FIG. 1F). Taken together, these data confirm that MAAP is a novel AAV protein predicted to contain cationic amphipathic C-terminal domain for membrane anchoring.

### Example 5

MAAP Ablation does not Affect AAV Capsid Protein Composition and Morphology

Recombinant AAV8 and AAV8 MAAPΔ virus was purified from the media of HEK293 producing cells. AAV8 and AAV8 MAAPΔ viral capsids were analyzed by SDS-PAGE under reducing conditions and stained with coomassie or probed with a capsid specific antibody (B1). Following ablation of MAAP, recombinant AAV8 and AAV8 MAAPΔ did not show a difference in protein content of viral capsids (FIG. 1G). Similarly, following MAAP ablation, the AAV8 and AAV8 MAAPΔ showed similar amounts of viral capsids using the monoclonal antibody B1 (FIG. 1H). TEM images of viral capsids from rAAV8 (FIG. 1I) and rAAV8 MAAPΔ (FIG. 1J) show that MAAP ablation did not affect the morphology of the capsids. These data confirm that MAAP ablation does not interact with the AAV capsid or Assembly Activating Protein (AAP).

### Example 6

### MAAP Plays a Role in the Synthesis of AAVs

Whether MAAP played a role in the synthesis of (i) (pseudo)wild type AAV serotype 8 (i.e., wtAAV8 packaging AAV2 rep and AAV8 cap flanked by AAV2 inverted terminal repeats [ITRs])) (FIG. 2A) and (ii) recombinant AAV8 (i.e., rAAV8 packaging a chicken beta-actin promoter driven luciferase transgene flanked by AAV2 ITRs)(FIG. 2D) was then determined. To ablate MAAP expression, the CTG start codon in the MAAP alternative open reading frame (ORF) was mutated without affecting the VP1 ORF in both wtAAV8 and rAAV8 plasmids.

Culture media and cell pellets were harvested following co-transfection with an Adenovirus helper plasmid (and an additional ITR flanked luciferase encoding transgene cassette in case of rAAV) on days 3 and 5 post-transfection. Strikingly, quantitative PCR of viral genomes revealed a significantly higher (˜1 log) amount of extracellular wtAAV8 particles in contrast to MAAPΔ particles recovered from media on day 3 post-transfection (FIG. 2B). Delayed secretion of the MAAPΔ particles, which were equally apportioned between extracellular and cell lysate fractions on day 5 post-transfection, was also observed. Although statistically significant, overall viral titers on day 5 post-transfection were only minimally altered. Of the total virus produced, nearly 70% of wtAAV8 particles were secreted by day 3 post-transfection, while MAAPΔ particles recovered in the extracellular fraction comprised <10% of total (FIG. 2C).

A similar trend was observed with the rAAV8 particles with a 4-5 fold higher recovery from media over cell lysate and delayed secretion in case of MAAPΔ particles (FIG. 2E). Of the total virus produced, ˜60% of rAAV8 particles were secreted by day 3 post-transfection in contrast to <10% of MAAPΔ particles (FIG. 2F). Thus, ablation of MAAP expression resulted in a significant delay in the extracellular secretion of wild type and recombinant AAV8 particles.

Further evaluation of AAV capsid proteins—VP1, VP2, and VP3—by western blot confirmed these results with undetectable to relatively lower levels in the extracellular fraction on day 3 post-transfection (FIG. 2G) and day 5 post-transfection (FIG. 2H), respectively, and correspondingly high(er) cellular retention in case of MAAPΔ particles. Moreover, no differences were observed when comparing the transduction efficiency of rAAV8 and MAAPΔ particles in vitro (FIG. 2I). Furthermore, MAAPΔ recombinant virus showed similar VP1, VP2, and VP3 expression ratios and overall virus morphology compared to rAAV8 (FIG. 1G-FIG. 1J). Taken together, these results show that encoding MAAP from the alternative ORF in VP1 is essential for efficient cellular egress of AAV particles.

Interestingly, ablation of MAAP expression differentially impacts recombinant AAV9 secretion. For example, there was a relatively low recovery of recombinant AAV serotype 9 (rAAV9) (˜15%) and MAAPΔ particles (˜5%) from media on day 3 post-transfection (FIG. 2J-FIG. 2K). Unlike rAAV8, rAAV9 particles had delayed secretion with only a modest difference in cellular egress efficiency compared to MAAPΔ particles. Thus, AAV8 appears more dependent on its cognate MAAP for cellular egress than is AAV9.

Collectively, these data demonstrate that MAAP plays a significant role in mediating vesicular egress of AAV from host cells. Cellular egress of both wild type and recombinant AAV particles is markedly attenuated when the MAAP ORF start site is mutated (MAAPΔ) with increased retention of AAV/MAAPΔ particles within the cell and accompanied by a significant delay in extracellular secretion. In other words, the ablation of MAAP expression results in a significant delay in the extracellular secretion of wild-type and recombinant AAV8 particles.

### Example 7

### Regions of MAAP8 are Critical for Expression and AAV Secretion

Whether specific regions of MAAP8 are necessary for expression and AAV secretion was examined. First, a sequence alignment of different MAAP mutants was done, which shows the alignment of the N-terminus, linker, and C-terminus of MAAP from AAV8. FIG. 3A shows the targeted deletion in each identified MAAP8 construct. For example, MAAP8 ΔN is missing the N-terminus, MAAP8 ΔC is missing the C-terminus, MAAP8 ΔL is missing the linker, and MAAP8 Δ NL is missing both the N-terminus and the linker. All MAAP mutants have a 3×-FLAG tag at the C terminus. Table 3 below shows the MAAP8 mutants shown in FIG. 3A and its sequence identifier.

TABLE 3
MAAP8 Mutants and Sequence Identifiers

| MAAP8 Mutant | Sequence Identifier |
|---|---|
| MAAP8 | SEQ ID NO: 08 |
| MAAP8 ΔN | SEQ ID NO: 36 |
| MAAP8 ΔL | SEQ ID NO: 37 |
| MAAP8 ΔNL | SEQ ID NO: 38 |
| MAAP8 ΔC | SEQ ID NO: 39 |
| MAAP8 Δ1 | SEQ ID NO: 40 |
| MAAP8 Δ2 | SEQ ID NO: 41 |
| MAAP8 Δ3 | SEQ ID NO: 42 |
| MAAP8 Δ4 | SEQ ID NO: 43 |
| MAAP8 Δ5 | SEQ ID NO: 44 |
| MAAP8 Δ6 | SEQ ID NO: 45 |
| MAAP8 Δ7 | SEQ ID NO: 46 |
| MAAP8 Δ1-2 | SEQ ID NO: 47 |
| MAAP8 Δ1-3 | SEQ ID NO: 48 |
| MAAP8 Δ1-4 | SEQ ID NO: 49 |

Second, as shown in FIG. 3B and FIG. 3C, anti-FLAG immunoblots of whole-cell extracts prepared from HEK293 cells expressing various MAAP8-3×-FLAG tagged constructs were examined and an anti-actin immunoblot served as the loading control. Third, as shown in FIG. 3D and FIG. 3E, recombinant MAAP8Δ vectors complemented in trans with various truncated MAAP8-3×-FLAG plasmids were analyzed from the media and pellet of HEK293 producing cells at day 3 post-transfection. Capsid proteins were analyzed by SDS-PAGE under reducing conditions and probed with a capsid (B1) specific antibody. Fourth, the total vector genomes was determined for various MAAP8 constructions in both the media and the cells (FIG. 3F) while the proportion of vector found in the media and cells 3 days post-transfection was also determined (FIG. 3G). In FIG. 3F and FIG. 3G, each bar is a representation of three experiments that are biological replicates and the error bar indicates a standard deviation from the mean. Collectively, these data demonstrate that MAAP retains functionality despite various deletions and/or perturbations to its sequence.

### Example 8

### Trans-Complementation Rescues MAAP Ablation

To determine whether MAAP expression regulates the secretion of other AAV serotypes, the CTG start codon in the MAAP ORF was mutated for rAAV1, rAAV2, rAAV8, and rAAV9, and viral titers in extracellular and cellular fractions at day 3 post-transcription were determined as described earlier. In parallel, whether MAAP trans-complementation could rescue the extracellular secretion of MAAPΔ rAAV particles was also evaluated. To achieve the latter, MAAP alone was expressed from the AAV helper plasmid containing rep and cap genes by mutating the start codons in the VP1, VP2, and VP3 as well as AAP ORFs. Strikingly, viral titers associated with the cellular fraction were markedly increased for rAAV1, rAAV8, and rAAV9 (˜4 to 7 fold), but not rAAV2 (FIG. 4A, FIG. 4C, FIG. 4E, and FIG. 4G). In addition, overall recovered titers were increased moderately for the same serotypes (up to 2 fold).

In corollary, a striking impact of ablating or supplementing MAAP expression on extracellular vs cell-associated fractions of different AAV serotypes was observed. Specifically, with respect to AAV1, this percentage was reversed from 60:40 to 20:80 upon MAAP ablation, and then restored to normal upon MAAP expression (FIG. 4B). A similar trend was observed for rAAV8 (˜80:20 to 20:80 followed by restoration to normal upon supplementation with WT MAAP) (FIG. 4F). Both rAAV2 and rAAV9 showed decreased secretion in general (˜35:65) when compared to rAAV1 and rAAV8 with MAAP ablation further reducing extracellular viral titers to 15% (rAAV2) and 20% (rAAV9). (FIG. 4D and FIG. 4H, respectively).

MAAP8 trans-complementation not only fully rescued the extracellular secretion of rAAV1, rAAV2, and rAAV8 particles, but also doubled the recovery of rAAV9 MAAPΔ particles from media as compared to rAAV9 particles (from 40% to 80%) (FIG. 4H). Trans-complementation with recombinant MAAP restored cellular egress of multiple AAV/MAAPΔ serotypes and increased the homogeneity of EV-associated viral particles. Mechanistically, MAAP appeared to preferentially associate with recycling Rab11⁺ vesicles over endo-lysosomal Rab7⁺ vesicles and co-localized with CD81⁺ vesicular fractions. These results confirm the critical role played by MAAP in enabling extracellular secretion of AAV particles in a serotype-independent manner albeit with different efficiencies. Further, these results demonstrate that trans-complementation of MAAP derived from AAV8 not only rescues secretion of different AAV serotypes, but also potentially enhance the kinetics of secretion.

### Example 9

### MAAP Mediates Cellular Egress of AAV Particles Through the Exosomal Pathway

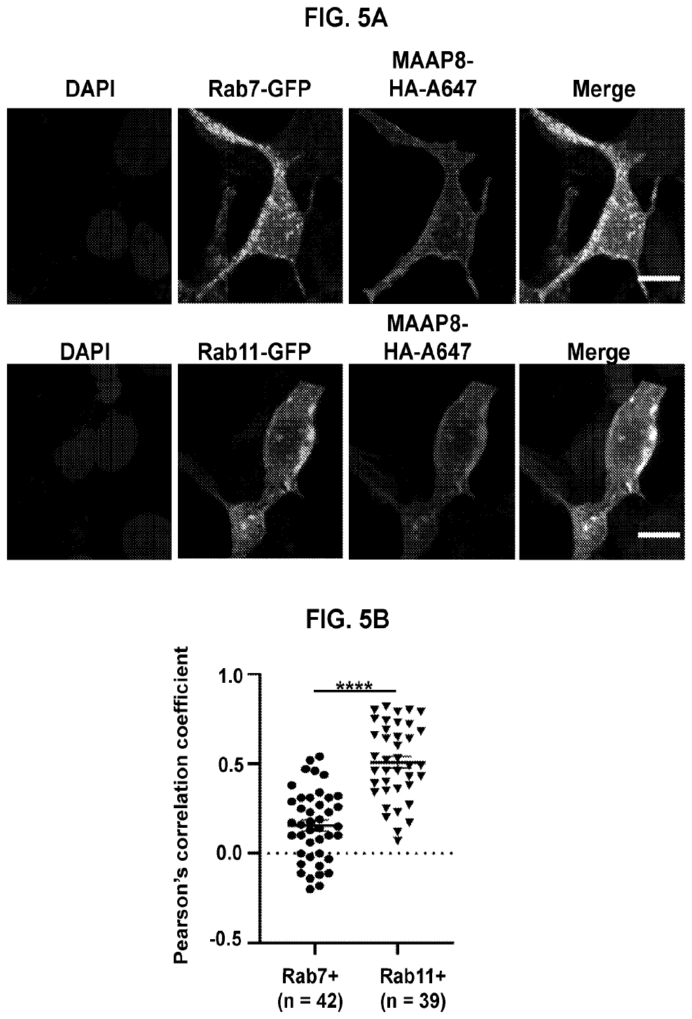

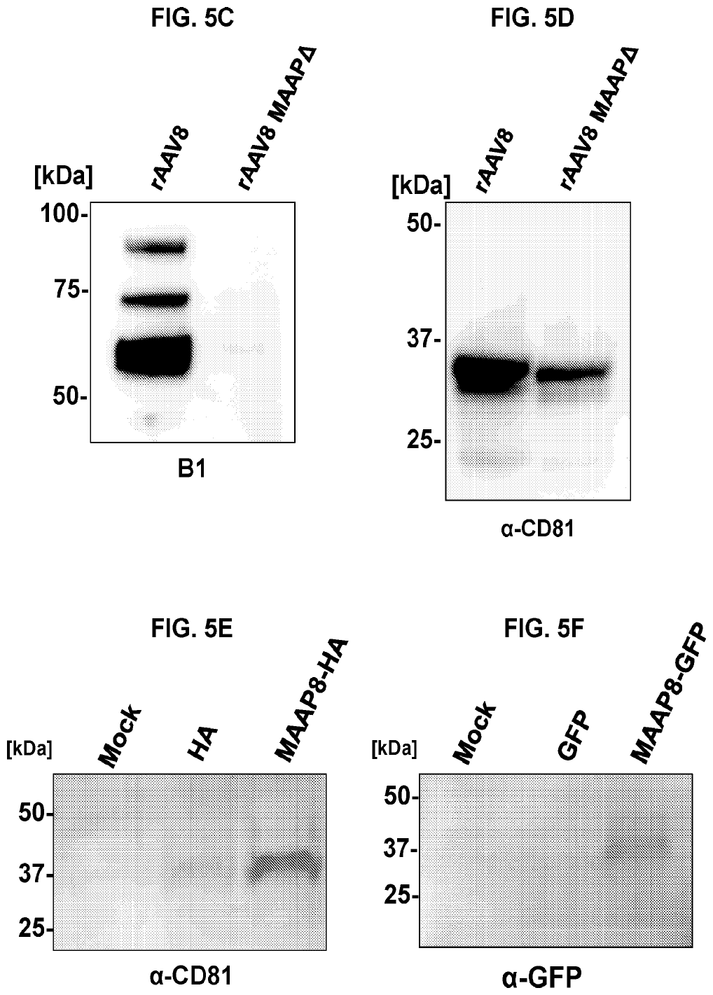

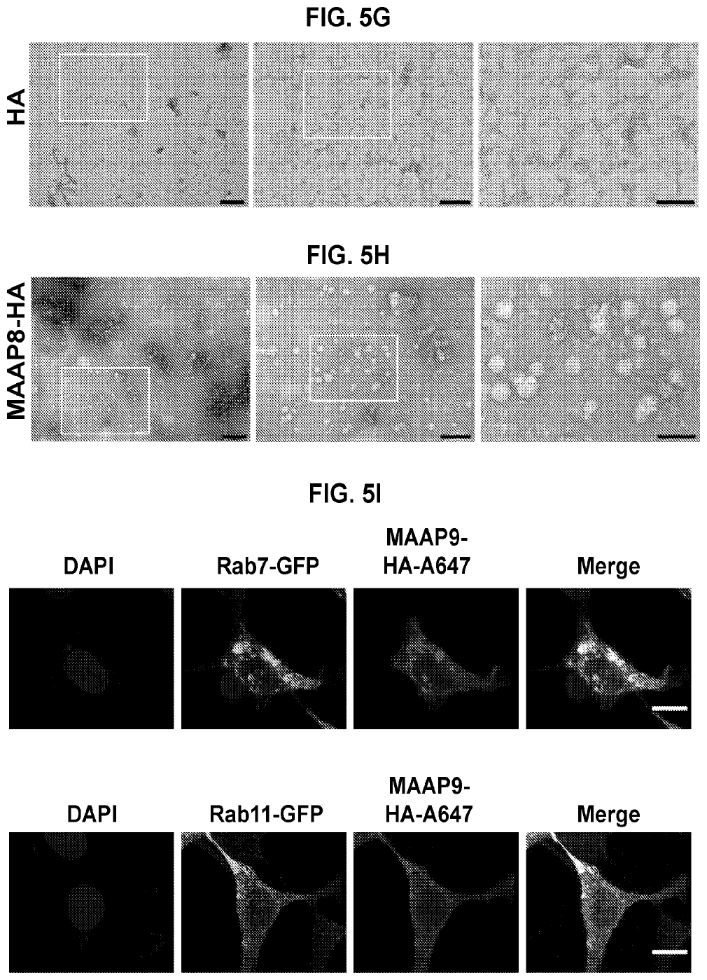

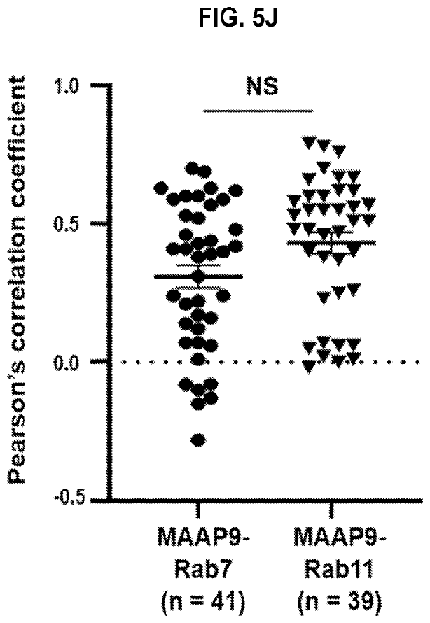

To understand how MAAP enables extracellular secretion of AAV particles, quantitative confocal fluorescence microscopy of cells overexpressing hemagglutinin (HA) tagged MAAP8 was performed. These results were then confirmed separately by western blot analysis of the various MAAP-HA constructs. (FIG. 4I). MAAP8-HA co-localized significantly more with the exosomal biogenesis pathway marker Rab11 (Koles K, et al. 2012 J Biol Chem. 287(20):16820-16834; Savina A. et al. 2002 J Cell Sci. 115(12):2505-2515; Savina A. et al. (2005) Traffic. 6(2):131-143), than the late endo/lysosomal pathway marker Rab7 (Shearer U, et al. 2019)(FIG. 5A and FIG. 5B).

MAAP9, which was associated earlier with slower secretion kinetics, showed a similar co-localization to both Rab7 and Rab11 subcellular compartments (FIG. 5I and FIG. 5J).

Overall, these results underscore the finding that MAAP is a key viral factor that mediates cellular egress of AAV particles through the exosomal pathway, an observation reported by others (Maguire C A, et al. 2012; György B, et al. 2017; Meliani A, et al. 2017 Blood Adv. 1(23):2019-2031; Hudry E, et al. 2016 Gene Ther. 23(4):380-392; Schiller L T, et al. 2018 Mol Ther Methods Clin Dev. 9:278-287; Orefice N S, et al. 2019 Mol Ther Methods Clin Dev. 14:237-251). Moreover, these results highlight potential differences in the structural attributes of different MAAPs that may explain the ability to exploit distinct secretory mechanisms that enables cellular egress of different AAV serotypes.

To further explore the biology of the MAAP-dependent AAV secretory process, purified EVs from the extracellular fractions of rAAV8 or rAAV8/MAAPΔ producing cells. In contrast to the rAAV8 control, negligible association of AAV particles associated with the purified EV fraction were observed as evidenced by western blot analysis of capsid proteins (FIG. 5C). A markedly lower signal for the exosome marker CD81 (Escola J M, et al. 1998 J Biol Chem. 273(32):20121-20127; Mathivanan S, et al. 2009 Proteomics. 9(21):4997-5000; Keerthikumar S, et al. 2016 J Mol Biol. 428(4):688-692) was observed in the rAAV8/MAAPΔ samples compared to rAAV8 controls (FIG. 5D).

### Example 10

### MAAP Overexpression Promotes Secretion of a Specific Type of Extracellular Vesicle

These results prompted the exploration of the function of MAAP outside of AAV biology. First, the overexpression of MAAP-HA when compared to an HA only control yielded a significantly higher proportion of CD81⁺ exosomal fraction (FIG. 5E). Second, when overexpressing MAAP8-GFP or a GFP only control, the former was significantly enriched in the purified exosomal fraction, which directly corroborated the role of MAAP in promoting the secretion of exosomes/EVs (FIG. 5F). Lastly, ultrastructural characterization of purified EV fractions using negative stain transmission electron microscopy (TEM) under different conditions (rAAV8, rAAV8/MAAPΔ, rAAV8/MAAPΔ+MAAP trans-complementation) revealed striking differences in the morphology, homogeneity, and abundance of exosomes (FIG. 5G). Particularly. MAAP overexpression was associated with a notable increase in spherical vesicles with a diameter ranging from ˜20-50 nm. Interestingly, these vesicles appeared to be relatively uniform and homogenous in composition, which indicated that MAAP can promote secretion of a specific type of extracellular vesicle.

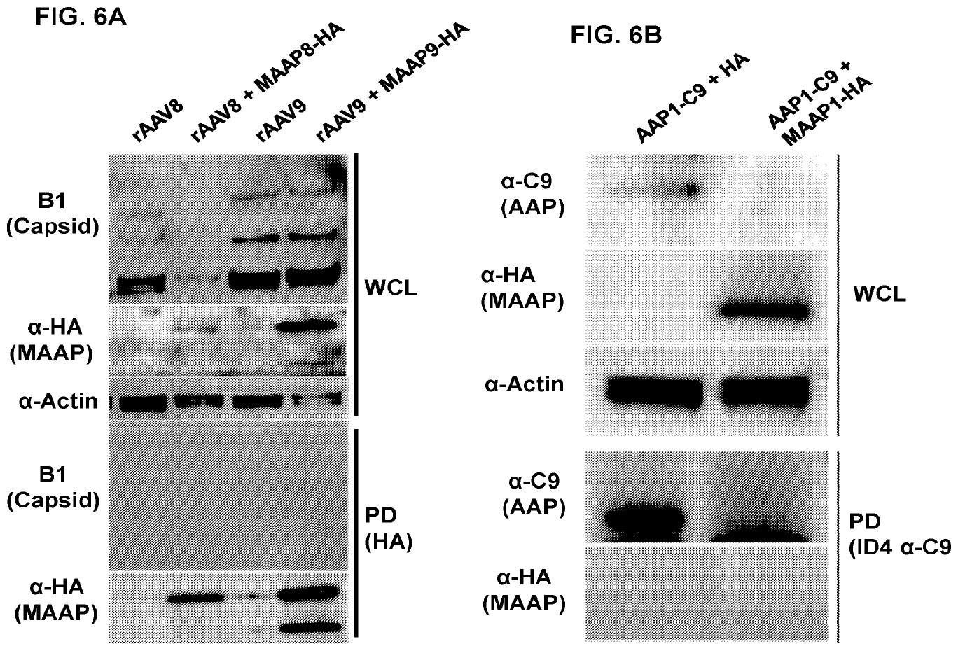

These studies did not yield any evidence of direct interaction between MAAP and AAV capsid proteins or MAAP and AAP as determined by immunoprecipitation analysis (FIG. 6A and FIG. 6B). Rather, the data indicated that MAAP likely exploits molecular interactions with the exosomal pathway instead.

### Example 11

MAAP Proximally Interacts with the AAV Capsid

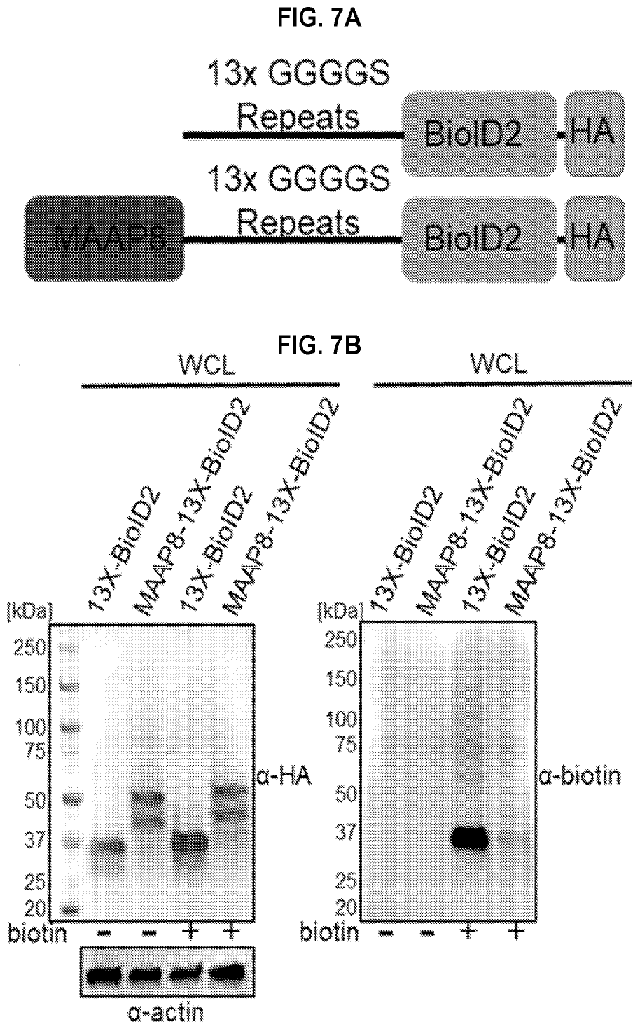

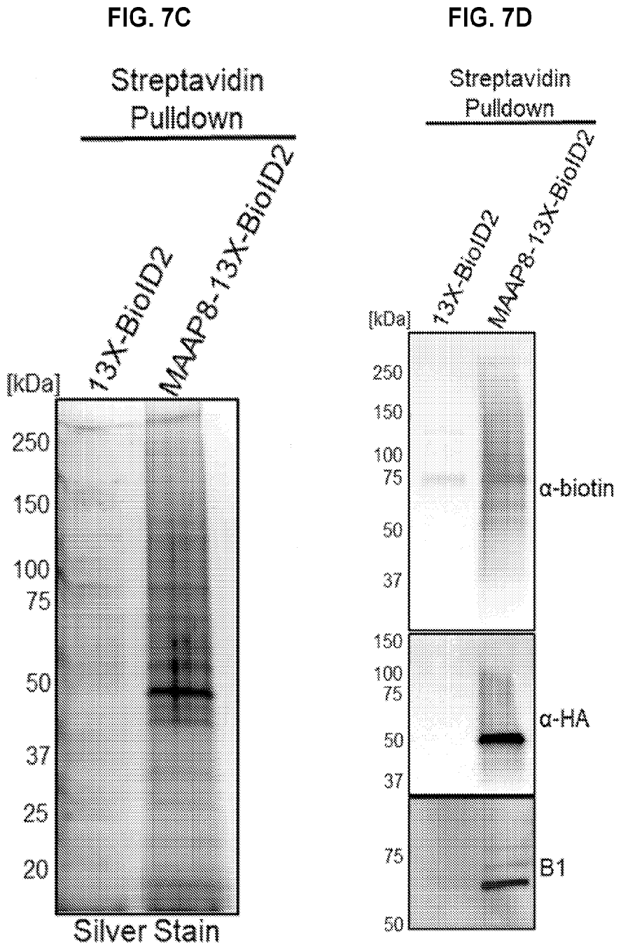

To detect protein-protein associations as well as proximate proteins in living cells, the BioID2 system was used. BiolD2 is a substantially smaller promiscuous biotin ligase, which enables more-selective targeting of fusion proteins, requires less biotin supplementation, and exhibits enhanced labeling of proximate proteins. Thus, BiolD2 improves the efficiency of screening for protein-protein associations. (Kim D I, et al. (2016) Mol Biol Cell. 27(8):1188-1196). FIG. 7A shows a schematic of MAAP8-13×-BiolD2-HA fusions. HEK293 cells were transfected with expression vectors encoding 13×-BiolD2 and MAAP8-13×-BiolD2. The media for these HEK293 cells was supplemented with 50 μM biotin 24 hours post-transfection and then the cells were harvested 24 hours post-biotin supplementation. Whole cell lysate (WCL) was analyzed by SDS-PAGE under reducing conditions and probed with HA (α-HA), biotin (α-biotin), and actin (α-actin) specific antibodies. (FIG. 7B). Next, HEK293 cells were transfected with plasmids encoding either 13×-BiolD2 or MAAP8-13×-BioID2 along with pXX680, pTR-CBA-Luciferase, and AAV8-MAAPΔ. Media for the HEK293 cells was supplemented with 50 μM biotin 48 hours post-transfection and cells were harvested 20 hours post-biotin supplementation. The biotinylated proteins pulled down on streptavidin resin were separated by SDS-PAGE and visualized by silver stain (FIG. 7C) or probed with biotin (α-biotin), (α-HA), and capsid (B1) specific antibodies (FIG. 7D). Collectively, these data show that MAAP interacts proximally with the AAV capsid.

### Example 12

### MAAP8-SACAS9 Fusions are Expressed In Vitro

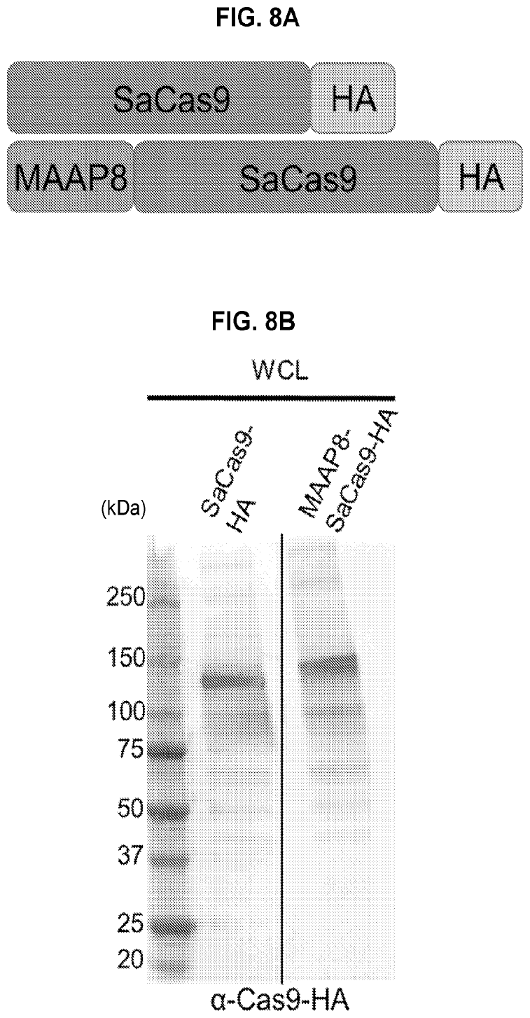

MAAP was fused with Cas9 (a CRISPR based-RNA guided nuclease commonly used for for gene and epigenome editing) from Staphylococus aureus with an HA tag (FIG. 8A). A control construct having SaCas9 with an HA tag was also created (FIG. 8B). Anti-Cas9-HA immunoblot of whole-cell lysates (WCL) prepared from HEK293 cells confirmed expression of the SaCas9-HA tagged constructs (FIG. 8B).

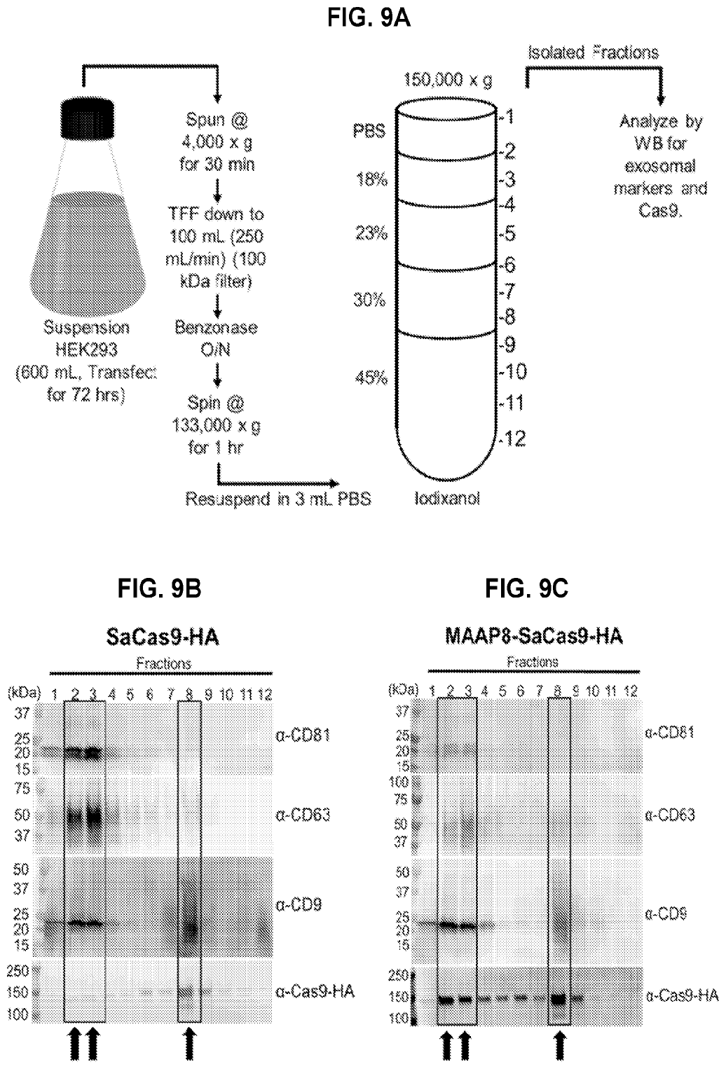

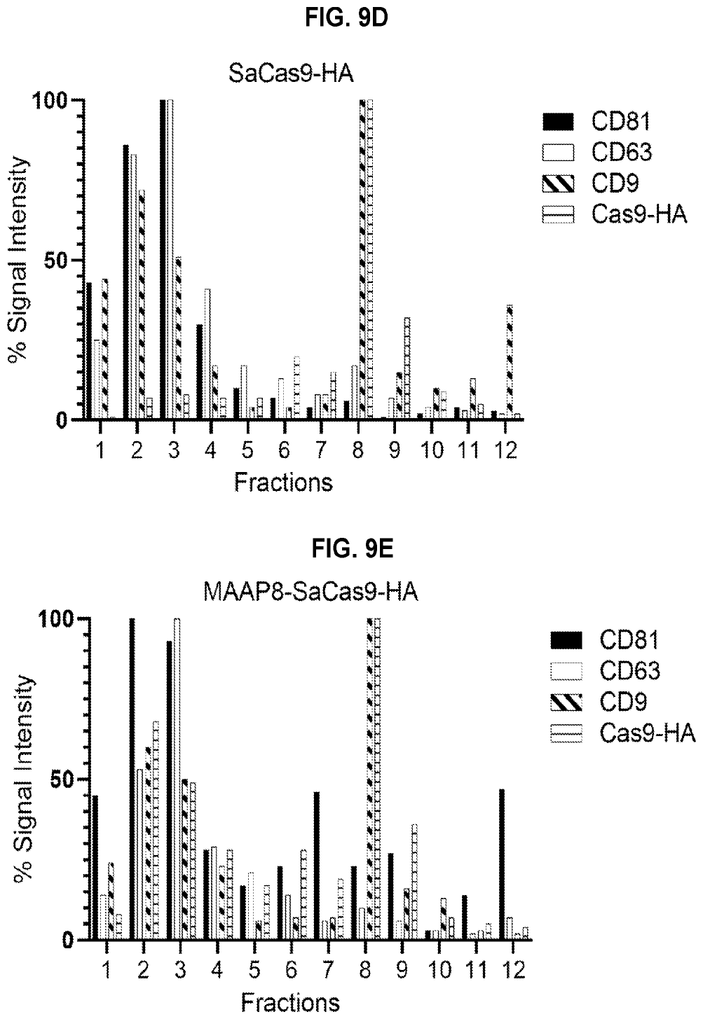

Then, after transfection of different constructs into HEK293 producer cells, media supernatant was subject to iodixanol gradient ultracentrifugation and different fractions separated. Each fraction was subject to immune detection of plasma membrane biomarkers that are used to identify exosomal fractions (CD63, CD9, and CD381) and co-detection of HA-tagged Cas9 in the same fractions. FIG. 9A provides a schematic highlighting methodology utilized for exosome isolation and characterization. Anti-CD81, CD63, CD9, and Cas9-HA immunoblots of individual iodixanol fractions from the conditioned media of HEK cells transfected with SaCas9-HA (FIG. 9B) and MAAP8-SaCas9-HA (FIG. 9C). A quantitative analysis of exosomal and Cas9 markers in individual iodixanol fractions was then performed for conditioned media of HEK cells transfected with SaCas9-HA (FIG. 9D) and MAAP8-SaCas9-HA (FIG. 9E). Signal intensity normalized to maximum intensity of each individual marker. Here, the densitometric analysis of the bands in the different fractions subject to western blot analysis corroborated the co-localization and enrichment of MAAP-Cas9-HA with exosomal fractions.

### Example 13

MAAP8 Enables SACAS9 Loading into Exosomes

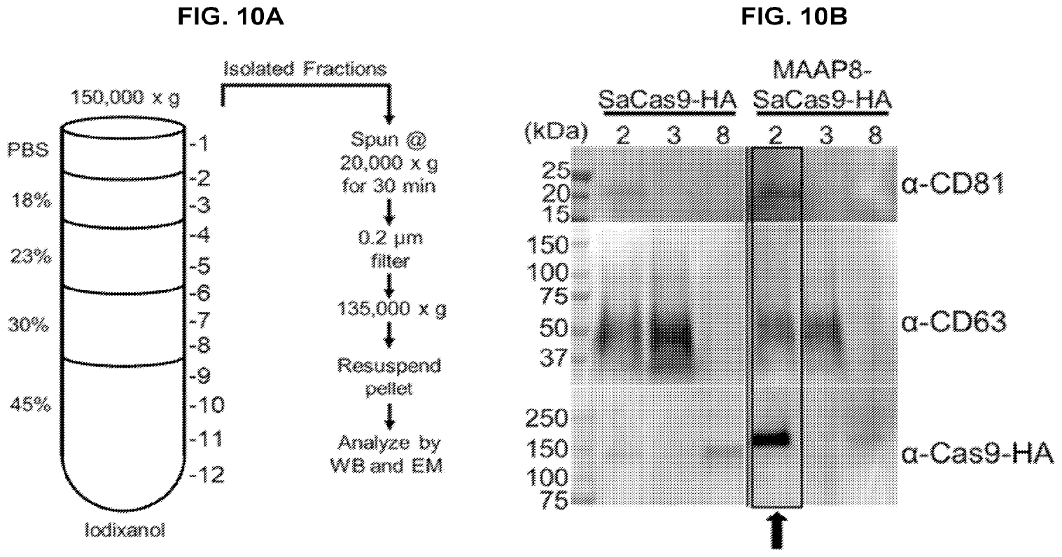

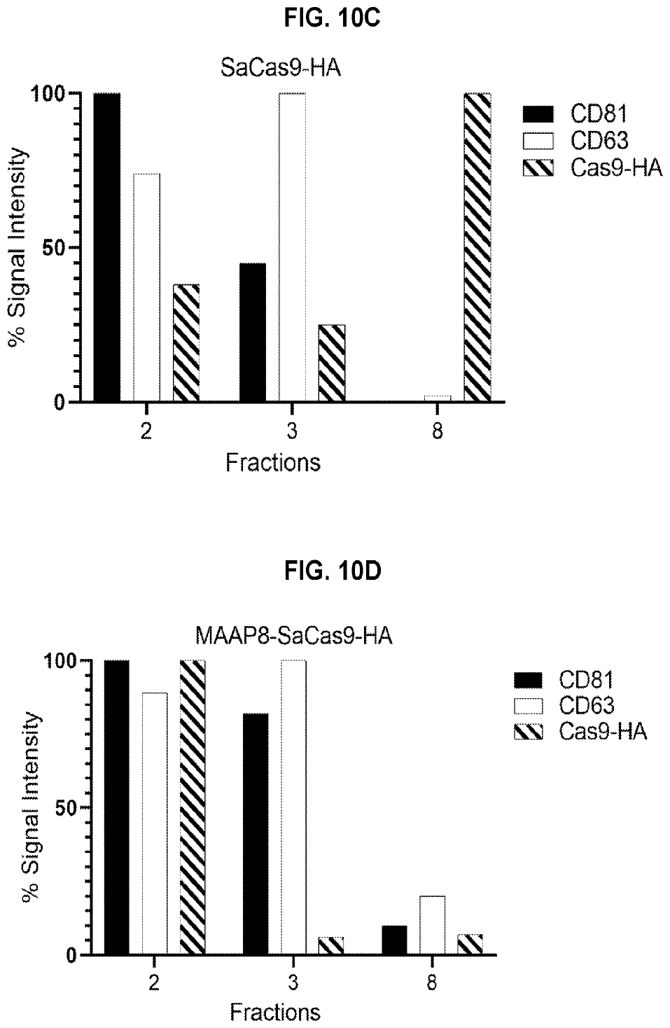

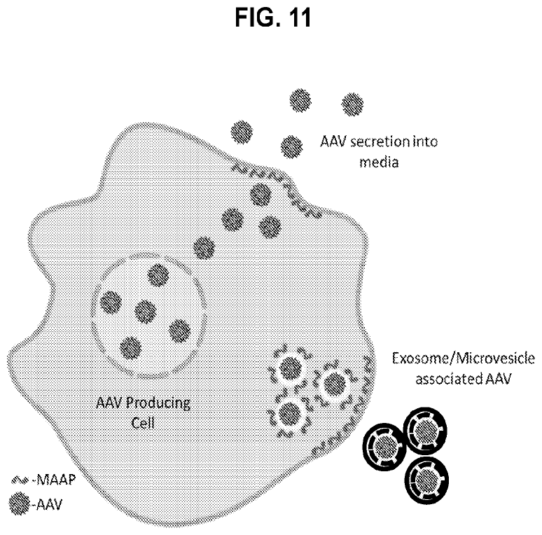

Whether MAAP8 enabled the loading of SaCas9 into exosomes was examined. FIG. 10A shows a schematic detailing the downstream processing of exosome containing iodixanol fractions. FIG. 10B shows anti-CD81, CD63, and Cas9-HA immunoblots of indicated processed iodixanol fractions from the conditioned media of HEK293 cells transfected with SaCas9-HA and MAAP8-SaCas9-HA. The quantitative analysis of exosomal and Cas9 markers in individual processed iodixanol fractions from the conditioned media of HEK293 cells transfected with SaCas9-HA (FIG. 10C) and MAAP8-SaCas9-HA (FIG. 10D) is shown. Signal intensity was normalized to maximum intensity of each individual marker. These data demonstrated a strong association between exosomal and Cas9-HA markers in fraction 2, which indicated that MAAP8-Cas9 was loaded into exosomes. Collectively, these data show that MAAP enabled selective loading of CRISPR/Cas9 cargo into exosomes as compared to passive loading methods utilized currently.

In summary, these data unequivocally show that MAAP is a novel AAV egress factor.

TABLE 4
Amino Acid and Nucleic Acid Sequences

| SEQ | |
|---|---|
| ID NO | SEQUENCE |
| 1 | LEPRSPKPTSKSRTTAGVWCFLATSTSDPSTDSTRGSPSTRRTQRPSSTTRPTTSSSKRVTIRTCGITTPTPSFRSVCK |
| | KIRLLGATSGEQSSRPRSGFSNLSVWLRKALRRLLERNVR |
| 2 | LAHHHQSPQSGIRTTAGVLCFLGTSTSDPSTDSTRESRSTRQTPRPSSTTKPTTGSSTAETTRTSSTTTPTRSFRSALK |
| | KIRLLGATSDEQSSRRKRGFLNLWAWLRNLLRRLREKRGR |
| 3 | LESLNPKRTNNTRTTVGVLCFRVTNTSDPVTDSTKESRSTRRTRQPSNTTKLTTSSSRPVTTRTSSTTTPTPSFRSVF |
| | KKIRLLGATLAEQSSRPKRGSLSLLVWLRKQLKRLLERRGL |
| 4 | LEPLNPRQINNIRTTLGVLCFRVTNTSDPATDSTRGNPSTQRTRQPSSTTRPTTSSSRPVTTPTSSTTTPTRSSSSGFR |
| | ATHRLGATSAEQSSRPKRGFLNLLVWLSKRVRRLLERRDR |
| 5 | RAHRNQNPISSIKIKPVVLCCLVITISDPETVSIEESLSTGQTRSRESTTSRTTSSLRRETTPTSSTTTRTPSFRRSSPTT |
| | HPSGETSERQSFRPRKGFSNLLAWLKRVLRRPLPESG |
| 6 | LEPRNPKPTSKSRTTAGVWCFLATSTSDPSTDSTRGSPSTRRMQRPSSTTRPTTSSSKRVTIRTCGITTPTPSFRSVC |
| | KKIRLLGATSGEQSSRPRRGFSNLLVWLRKVLRRLLERNVR |
| 7 | LEPRNPKPTSKSRTTAGVWCFLATSTSDPSTDSTRGSPSTRRTQRPSSTTRPTTSSSKRVTIRTCGITTPTPSFRSVCK |
| | KIRHLGATSGEQSSRPRSGFSNLSVWLRKALRRLLQRRDR |
| 8 | LEPRSPKPTSKSRTTAGVWCFLATSTSDPSTDSTRGSPSTRRTQRPSSTTRPTTSSCRRVTIRTCGITTPTPSFRSVCK |
| | KIRLLGATSGEQSSRPRSGFSNLSVWLRKALRRLLERRDR |
| 9 | LEPLNPRQINNIKTTLEVLCFRVTNTLDPATDSTRGSRSTQQTRRPSSTTRPTTSSSRPETTRTSSTTTPTPSSRSGSK |
| | KIRLLGATSGEQSSRPKRGFLNLLVWLRKRLRRLLERRGL |
| 10 | LEPPSPRPTSRSRTTAGVWCFLATSTSDPSTDSTRGSPSTRRTQRPSSTTRPTTSSSKRVTIRTCGITTPTPSFRSVCK |
| | KIRLLGATSGEQSSRPRSGFSNLSVWLRKLLRRLLERRDR |
| 11 | LEPRSPRPTSRSRTTAGVWCFLATSTSDPSTDSTRGSPSTRRTQRPSSTTRPTTSSSKRVTIRTCGITTPTPSERSVCK |
| | KIRLLGATSGEQSSRPRRGYSNLWAWLKKVLKRLLERRDR |
| 12 | LELHNPRPTNSIRTTAGVLCFLGTSTSDPSTDSTRESRSTRQTPRPSSTTRPTTSSSSRGTTRISSTTTPTPSSSSAWRP |
| | TPLLGATSGEQSSRPKRGFSSLWVWLKRALKRLLERNAH |
| 13 | LEPLNPRQINNIRTTLGVLCFRVTNTSDPATDLTRGNPSTQRTRQPSNTTRPTTSSSRPVTTPTSSTTTPTPSFRSVFK |
| | KIRLLGATSDEQSSRPKRGSLSLWVWLRKRLRRLLEKRDL |
| 14 | LEPRNPKPTSKSRTTAGVWCFLATSTSDPSTDSTRGSPSTRRTQRPSSTTKPTTSSSKRVTIRTCGITPTPSFRSVCK |
| | KIRLLGATSGEQSSRPRSGFSNLSVWLRKALRRLLERRDR |
| 15 | LEPRNPKPTSKSRTTAGVWCFLATSTSDPSTDSTRGSPSTRRTQRPSSTTRPTTSSSKRVTIRTCGITTPTPSFRSVCK |
| | KIRLLGATSGEQSSRPRSGFSNLSVWLRKALRRLLERRDR |
| 16 | atggctgccgatggttatcttccagattggctcgaggacaacctctctgagggcattcgcgagtggtgggacttgaaacNNNgagccccgaagcccaaagccaaccagcaaa |
| | agcaggacgacggccggggtctggtgcttcctggctacaagtacctcggacccttcaacggactcgacaagggggagcccgtcaacgcggcggacgcagcggccctcgagc |
| | acgacaaggcctacgaccagcagctcaaagcgggtgacaatccgtacctgcggtataaccacgccgacgccgagtttcaggagcgtctgcaagaagatacgtcttttggggg |
| | aacctcgggcgagcagtcttccaggccaagaagcgggttctcgaacctctcggtctggttgaggaaggcgctaagacggctcctggaaagaaacgtccggtagagcagtcgcc |
| | acaagagccagactcctcctcgggcatcggcaagacaggccagcagcccgctaaaaagagactcaattttggtcagactggcgactcagagtcagtccccgatccacaacctct |
| | cggagaacctccagcaacccccgctgctgtgggacctactacaatggcttcaggcggtggcgcaccaatggcagacaataacgaaggcgccgacggagtgggtaatgcctca |
| | ggaaattggcattgcgattccacatggctgggcgacagagtcatcaccaccagcacccgcacctgggccttgcccacctacaataaccacctctacaagcaaatctccagtgcttc |
| | aacgggggccagcaacgacaaccactacttcggctacagcaccccctgggggtattttgatttcaacagattccactgccacttttcaccacgtgactggcagcgactcatcaaca |
| | acaattggggattccggcccaagagactcaacttcaaactcttcaacatccaagtcaaggaggtcacgacgaatgatggcgtcacaaccatcgctaataaccttaccagcacggtt |
| | caagtcttctcggactcggagtaccagcttccgtacgtcctcggctctgcgcaccagggctgcctccctccgttcccggcggacgtgttcatgattccgcaatacggctacctgacg |
| | ctcaacaatggcagccaagccgtgggacgttcatccttttactgcctggaatatttcccttctcagatgctgagaacgggcaacaactttaccttcagctacacctttgaggaagtgcc |
| | tttccacagcagctacgcgcacagccagagcctggaccggctgatgaatcctctcatcgaccaatacctgtattacctgaacagaactcaaaatcagtccggaagtgcccaaaaca |
| | aggacttgctgtttagccgtgggtctccagctggcatgtctgttcagcccaaaaactggctacctggaccctgttatcggcagcagcgcgtttctaaaacaaaaacagacaacaaca |
| | acagcaattttacctggactggtgcttcaaaatataacctcaatgggcgtgaatccatcatcaaccctggcactgctatggcctcacacaaagacgacgaagacaagttctttcccat |
| | gagcggtgtcatgatttttggaaaagagagcgccggagcttcaaacactgcattggacaatgtcatgattacagacgaagaggaaattaaagccactaaccctgtggccaccgaa |
| | agatttgggaccgtggcagtcaatttccagagcagcagcacagaccctgcgaccggagatgtgcatgctatgggagcattacctggcatggtgtggcaagatagagacgtgtac |
| | ctgcagggtcccatttgggccaaaattcctcacacagatggacactttcacccgtctcctcttatgggcggctttggactcaagaacccgcctcctcagatcctcatcaaaaacacgc |
| | ctgttcctgcgaatcctccggcggagttttcagctacaaagtttgcttcattcatcacccaatactccacaggacaagtgagtgtggaaattgaatgggagctgcagaaagaaaaca |
| | gcaagcgctggaatcccgaagtgcagtacacatccaattatgcaaaatctgccaacgttgattttactgtggacaacaatggactttatactgagcctcgccccattggcacccgtta |
| | ccttacccgtcccctgtaa |
| 17 | atggctgccgatggttatcttccagattggctcgaggacactctctctgaaggaataagacagtggtggaagctcaaacNNNgcccaccaccaccaaagcccgcagagcggc |
| | ataaggacgacagcaggggtcttgtgcttcctgggtacaagtacctcggacccttcaacggactcgacaagggagagccggtcaacgaggcagacgccgcggccctcgagca |
| | cgacaaagcctacgaccggcagctcgacagcggagacaacccgtacctcaagtacaaccacgccgacgcggagtttcaggagcgccttaaagaagatacgtcttttgggggca |
| | acctcggacgagcagtcttccaggcgaaaaagagggttcttgaacctctgggcctggttgaggaacctgttaagacggctccgggaaaaaagaggccggtagagcactctcctg |
| | tggagccagactcctcctcgggaaccggaaaggcgggccagcagcctgcaagaaaaagattgaattttggtcagactggagacgcagactcagtacctgacccccagccttc |
| | ggacagccaccagcagccccctctggtctgggaactaatacgatggctacaggcagtggcgcaccaatggcagacaataacgagggcgccgacggagtgggtaattcctcgg |
| | gaaattggcattgcgattccacatggatgggcgacagagtcatcaccaccagcacccgaacctgggccctgcccacctacaacaaccacctctacaaacaaatttccagccaatc |
| | aggagcctcgaacgacaatcactactttggctacagcaccccttgggggtattttgacttcaacagattccactgccacttttcaccacggactggcaaagactcatcaacaacaac |
| | tggggattccgacccaagagactcaacttcaagctctttaacattcaagtcaaagaggtcacgcagaatgacggtacgacgacgattgccaataaccttaccagcacggttcaggt |
| | gtttactgactcggagtaccagctcccgtacgtcctcggctcggcgcatcaaggatgcctcccgccgttcccagcagacgtcttcatggtgccacagtatggatacctcaccctgaa |
| | caacgggagtcaggcagtaggacgctcttcattttactgcctggagtactttccttctcagatgctgcgtaccggaaacaactttaccttcagctacacttttgaggacgttcctttccac |
| | agcagctacgctcacagccagagtctggaccgtctcatgaatcctctcatcgaccagtacctgtattacttgagcagaacaaacactccaagtggaaccaccacgcagtcaaggct |
| | tcagttttctcaggccggagcgagtgacattcgggaccagtctaggaactggcttcctggaccctgttaccgccagcagcgagtatcaaagacatctgcggataacaacaacagtg |
| | aatactcgtggactggagctaccaagtaccacctcaatggcagagactctctggtgaatccgggcccggccatggcaagccacaaggacgatgaagaaaagttttttcctcagag |
| | cggggttctcatctttgggaagcaaggctcagagaaaacaaatgtggacattgaaaaggtcatgattacagacgaagaggaaatcaggacaaccaatcccgtggctacggagca |
| | gtatggttctgtatctaccaacctccagagaggcaacagacaagcagctaccgcagatgtcaacacacaaggcgttcttccaggcatggtctggcaggacagagatgtgtaccttc |
| | aggggcccatctgggcaaagattccacacacggacggacattttcacccctctcccctcatgggggattcggacttaaacaccctcctccacagattctcatcaagaacaccccg |
| | gtacctgcgaatccttcgaccaccttcagtgcggcaaagtttgcttccttcatcacacagtactccacgggacaggtcagcgtggagatcgagtgggagctgcagaaggaaaaca |
| | gcaaacgctggaatcccgaaattcagtacacttccaactacaacaagtctgttaatgtggactttactgtggacactaatggcgtgtattcagagcctcgccccattggcaccagata |
| | cctgactcgtaatctgtaa |
| 18 | atggctgctgacggttatcttccagattggctcgaggacaacctttctgaaggcattcgtgagtggtgggctctgaaacNNNgagtccctcaacccaaagcgaaccaacaacac |
| | caggacaaccgtcggggtcttgtgcttccgggttacaaatacctcggacccggtaacggactcgacaaaggagagccggtcaacgaggcggacgcggcagccctcgaacacg |
| | acaaagcttacgaccagcagctcaaggccggtgacaacccgtacctcaagtacaaccacgccgacgccgagtttcaggagcgtcttcaagaagatacgtcttttgggggcaacc |
| | ttggcagagcagtcttccaggccaaaaagaggatccttgagcctcttggtctggttgaggaagcagctaaaacggctcctggaaagaagggggctgtagatcagtctcctcagga |
| | accggactcatcatctggtgttggcaaatcgggcaaacagcctgccagaaaaagactaaatttcggtcagactggagactcagagtcagtcccagaccctcaacctctcggagaa |
| | ccaccagcagcccccacaagtttgggatctaatacaatggcttcaggcggtggcgcaccaatggcagacaataacgagggtgccgatggagtgggtaattcctcaggaaattgg |
| | cattgcgattcccaatggctgggcgacagagtcatcaccaccagcaccagaacctgggccctgcccacttacaacaaccatctctacaagcaaatctccagccaatcaggagctt |
| | caaacgacaaccactactttggctacagcaccccttgggggtattttgactttaacagattccactgccacttctcaccacgtgactggcagcgactcattaacaacaactggggattc |
| | cggcccaagaaactcagcttcaagctcttcaacatccaagttagaggggtcacgcagaacgatggcacgacgactattgccaataaccttaccagcacggttcaagtgtttacgga |
| | ctcggagtatcagctcccgtacgtgctcgggtcggcgcaccaaggctgtctcccgccgtttccagcggacgtcttcatggtccctcagtatggatacctcaccctgaacaacggaa |
| | gtcaagcggtgggacgctcatccttttactgcctggagtacttcccttcgcagatgctaaggactggaaataacttccaattcagctataccttcgaggatgtaccttttcacagcagct |
| | acgctcacagccagagtttggatcgcttgatgaatcctcttattgatcagtatctgtactacctgaacagaacgcaaggaacaacctctggaacaaccaaccaatcacggctgcttttt |
| | agccaggctgggcctcagtctatgtctttgcaggccagaaattggctacctgggccctgctaccggcaacagagactttcaaagactgctaacgacaacaacaacagtaactttcct |
| | tggacagcggccagcaaatatcatctcaatggccgcgactcgctggtgaatccaggaccagctatggccagtcacaaggacgatgaagaaaaatttttccctatgcacggcaatct |
| | aatatttggcaaagaagggacaacggcaagtaacgcagaattagataatgtaatgattacggatgaagaagagattcgtaccaccaatcctgtggcaacagagcagtatggaact |
| | gtggcaaataacttgcagagctcaaatacagctcccacgactggaactgtcaatcatcagggggccttacctggcatggtgtggcaagatcgtgacgtgtaccttcaaggacctat |
| | ctgggcaaagattcctcacacggatggacactttcatccttctcctctgatgggaggctttggactgaaacatccgcctcctcaaatcatgatcaaaaatactccggtaccggcaaat |
| | cctccgacgactttcagcccggccaagtttgcttcatttatcactcagtactccactggacaggtcagcgtggaaattgagtgggagctacagaaagaaaacagcaaacgttggaat |
| | ccagagattcagtacacttccaactacaacaagtctgttaatgtggactttactgtagacactaatggtgtttatagtgaacctcgccctattggaacccggtatctcacacgaaacttgt |
| | ga |
| 19 | atgactgacggttaccttccagattggctagaggacaacctctctgaaggcgttcgagagtggtgggcgctgcaacNNNgagcccctaaacccaaggcaaatcaacaacatca |
| | ggacaacgctcggggtcttgtgcttccgggttacaaatacctcggacccggcaacggactcgacaagggggaacccgtcaacgcagcggacgcggcagccctcgagcacga |
| | caaggcctacgaccagcagctcaaggccggtgacaacccctacctcaagtacaaccacgccgacgcggagttccagcagcggcttcagggcgacacatcgtttgggggcaac |
| | ctcggcagagcagtcttccaggccaaaaagagggttcttgaacctcttggtctggttgagcaagcgggtgagacggctcctggaaagaagagaccgttgattgaatccccccagc |
| | agcccgactcctccacgggtatcggcaaaaaaggcaagcagccggctaaaaagaagctcgttttcgaagacgaaactggagcaggcgacggaccccctgagggatcaacttc |
| | cggagccatgtctgatgacagtgagatgcgtgcagcagctggcggagctgcagtcgagggcggacaaggtgccgatggagtgggtaatgcctcgggtgattggcattgcgatt |
| | ccacctggtctgagggccacgtcacgaccaccagcaccagaacctgggtcttgcccacctacaacaaccacctctacaagcgactcggagagagcctgcagtccaacacctac |
| | aacggattctccaccccctggggatactttgacttcaaccgcttccactgccacttctcaccacgtgactggcagcgactcatcaacaacaactggggcatgcgacccaaagccat |
| | gcgggtcaaaatcttcaacatccaggtcaaggaggtcacgacgtcgaacggcgagacaacggtggctaataaccttaccagcacggttcagatctttgcggactcgtcgtacgaa |
| | ctgccgtacgtgatggatgcgggtcaagagggcagcctgcctccttttcccaacgacgtctttatggtgccccagtacggctactgtggactggtgaccggcaacacttcgcagca |
| | acagactgacagaaatgccttctactgcctggagtactttccttcgcagatgctgcggactggcaacaactttgaaattacgtacagttttgagaaggtgcctttccactcgatgtacg |
| | cgcacagccagagcctggaccggctgatgaaccctctcatcgaccagtacctgtggggactgcaatcgaccaccaccggaaccaccctgaatgccgggactgccaccaccaa |
| | ctttaccaagctgcggcctaccaacttttccaactttaaaaagaactggctgcccgggccttcaatcaagcagcagggcttctcaaagactgccaatcaaaactacaagatccctgc |
| | caccgggtcagacagtctcatcaaatacgagacgcacagcactctggacggaagatggagtgccctgacccccggacctccaatggccacggctggacctgcggacagcaag |
| | ttcagcaacagccagctcatctttgcggggcctaaacagaacggcaacacggccaccgtacccgggactctgatcttcacctctgaggaggagctggcagccaccaacgccac |
| | cgatacggacatgtgggg ggggcaacctacctggcggtgaccagagcaacagcaacctgccgaccgtggacagactgacagccttgggagccgtgcctggaatggtctggcaaaac |
| | agagacatttactaccagggtcccatttgggccaagattcctcataccgatggacactttcacccctcaccgctgattggtgggtttgggctgaaacacccgcctcctcaaatttttatc |
| | aagaacaccccggtacctgcgaatcctgcaacgaccttcagctctactccggtaaactccttcattactcagtacagcactggccaggtgtcggtgcagattgactgggagatcca |
| | gaaggagcggtccaaacgctggaaccccgaggtccagtttacctccaactacggacagcaaaactctctgttgtgggctcccgatgcggctgggaaatacactgagcctagggc |
| | tatcggtacccgctacctcacccaccacctgtaa |
| 20 | atgtcttttgttgatcaccctccagattggttggaagaagttggtgaaggtcttcgcgagtttttgggccttgaagNNNgcccaccgaaaccaaaacccaatcagcagcatcaaga |
| | tcaagcccgtggtcttgtgctgcctggttataactatctcggacccggaaacggtctcgatcgaggagagcctgtcaacagggcagacgaggtcgcgcgagagcacgacatctc |
| | gtacaacgagcagcttgaggcgggagacaacccctacctcaagtacaaccacgcggacgccgagtttcaggagaagctcgccgacgacacatccttcgggggaaacctcgga |
| | aaggcagtctttcaggccaagaaaagggttctcgaaccttttggcctggttgaagagggtgctaagacggcccctaccggaaagcggatagacgaccactttccaaaaagaaag |
| | aaggctcggaccgaagaggactccaagccttccacctcgtcagacgccgaagctggacccagcggatcccagcagctgcaaatcccagcccaaccagcctcaagtttgggag |
| | ctgatacaatgtctgcgggaggtggcggcccattgggcgacaataaccaaggtgccgatggagtgggcaatgcctcgggagattggcattccgattccacgtggatgggggac |
| | agagtcgtcaccaagtccacccgaacctgggtgctgcccagctacaacaaccaccagtaccgagagatcaaaagcggctccgtcgacggaagcaacgccaacgcctactttgg |
| | atacagcaccccctgggggtactttgactttaaccgcttccacagccactggagcccccgagactggcaaagactcatcaacaactactggggcttcagaccccggtccctcaga |
| | gtcaaaatcttcaacattcaagtcaaagaggtcacggtgcaggactccaccaccaccatcgccaacaacctcacctccaccgtccaagtgtttacggacgacgactaccagctgcc |
| | ctacgtcgtcggcaacgggaccgagggatgcctgccggccttccctccgcaggtctttacgctgccgcagtacggttacgcgacgctgaaccgcgacaacacagaaaatccca |
| | ccgagaggagcagcttcttctgcctagagtactttcccagcaagatgctgagaacgggcaacaactttgagtttacctacaactttgaggaggtgcccttccactccagcttcgctcc |
| | cagtcagaacctgttcaagctggccaacccgctggtggaccagtacttgtaccgcttcgtgagcacaaataacactggcggagtccagttcaacaagaacctggccgggagatac |
| | gccaacacctacaaaaactggttcccggggcccatgggccgaacccagggctggaacctgggctccggggtcaaccgcgccagtgtcagcgccttcgccacgaccaatagga |
| | tggagctcgagggcgcgagttaccaggtgcccccgcagccgaacggcatgaccaacaacctccagggcagcaacacctatgccctggagaacactatgatcttcaacagcca |
| | gccggcgaacccgggcaccaccgccacgtacctcgagggcaacatgctcatcaccagcgagagcgagacgcagccggtgaaccgcgtggcgtacaacgtcggcgggcag |
| | atggccaccaacaaccagagctccaccactgcccccgcgaccggcacgtacaacctccaggaaatcgtgcccggcagcgtgtggatggagagggacgtgtacctccaagga |
| | cccatctgggccaagatcccagagacgggggcgcactttcacccctctccggccatgggcggattcggactcaaacacccaccgcccatgatgctcatcaagaacacgcctgtg |
| | cccggaaatatcaccagcttctcggacgtgcccgtcagcagcttcatcacccagtacagcaccgggcaggtcaccgtggagatggagtgggagctcaagaaggaaaactccaa |
| | gaggtggaacccagagatccagtacacaaacaactacaacgacccccagtttgtggactttgccccggacagcaccggggaatacagaaccaccagacctatcggaacccgat |
| | accttacccgacccctttaa |
| 21 | atggctgccgatggttatcttccagattggctcgaggacaacctctctgagggcattcggagtggtgggacttgaaacNNNgagccccgaaacccaaagccaaccagcaaa |
| | agcaggacgacggccggggtctggtgcttcctggctacaagtacctcggacccttcaacggactcgacaagggggagcccgtcaacgcggcggatgcagcggccctcgagc |
| | acgacaaggcctacgaccagcagctcaaagcgggtgacaatccgtacctgcggtataaccacgccgacgccgagtttcaggagcgtctgcaagaagatacgtcttttgggggc |
| | aacctcgggcgagcagtcttccaggccaagaagagggttctcgaaccttttggtctggttgaggaaggtgctaagacggctcctggaaagaaacgtccggtagagcagtcgcca |
| | caagagccagactcctcctcgggcattggcaagacaggccagcagcccgctaaaaagagactcaattttggtcagactggcgactcagagtcagtccccgacccacaacctctc |
| | ggagaacctccagcaacccccgctgctgtgggacctactacaatggcttcaggcggtggcgcaccaatggcagacaataacgaaggcgccgacggagtgggtaatgcctcag |
| | gaaattggcattgcgattccacatggctgggcgacagagtcatcaccaccagcacccgaacatgggccttgcccacctataacaaccacctctacaagcaaatctccagtgcttca |
| | acgggggccagcaacgacaaccactacttcggctacagcaccccctgggggtattttgatttcaacagattccactgccatttctcaccacgtgactggcagcgactcatcaacaa |
| | caattggggattccggcccaagagactcaacttcaagctcttcaacatccaagtcaaggaggtcacgacgaatgatggcgtcacgaccatcgctaataaccttaccagcacggttc |
| | aagtcttctcggactcggagtaccagttgccgtacgtcctcggctctgcgcaccagggctgcctccctccgttcccggcggacgtgttcatgattccgcagtacggctacctaacgc |
| | tcaacaatggcagccaggcagtgggacggtcatccttttactgcctggaatatttcccatcgcagatgctgagaacgggcaataactttaccttcagctacaccttcgaggacgtgc |
| | ctttccacagcagctacgcgcacagccagagcctggaccggctgatgaatcctctcatcgaccagtacctgtattacctgaacagaactcagaatcagtccggaagtgcccaaaa |
| | caaggacttgctgtttagccgggggtctccagctggcatgtctgttcagcccaaaaactggctacctggaccctgttaccggcagcagcgcgtttctaaaacaaaaacagacaaca |
| | acaacagcaactttacctggactggtgcttcaaaatataaccttaatgggcgtgaatctataatcaaccctggcactgctatggcctcacacaaagacgacaaagacaagttctttcc |
| | catgagcggtgtcatgatttttggaaaggagagcgccggagcttcaaacactgcattggacaatgtcatgatcacagacgaagaggaaatcaaagccactaaccccgtggccacc |
| | gaaagatttgggactgtggcagtcaatctccagagcagcagcacagaccctgcgaccggagatgtgcatgttatgggagccttacctggaatggtgtggcaagacagagacgta |
| | tacctgcagggtcctatttgggccasaattcctcacacggatggacactttcacccgtctcctctcatgggcggctttggacttaagcacccgcctcctcagatcctcatcaaaaacac |
| | gcctgttcctgcgaatcctccggcagagttttcggctacaaagtttgcttcattcatcacccagtattccacaggacaagtgagcgtggagattgaatgggagctgcagaaagaaaa |
| | cagcaaacgctggaatcccgaagtgcagtatacatctaactatgcaaaatctgccaacgttgatttcactgtggacaacaatggactttatactgagcctcgccccattggcacccgt |
| | tacctcacccgtcccctgtaa |
| 22 | atggctgccgatggttatcttccagattggctcgaggacaacctctctgagggcattcgcgagtggtgggacctgaaacNNNgagccccgaaacccaaagccaaccagcaaa |
| | agcaggacaacggccggggtctggtgcttcctggctacaagtacctcggacccttcaacggactcgacaagggggagcccgtcaacgcggcggacgcagcggccctcgagc |
| | acgacaaggcctacgaccagcagctcaaagcgggtgacaatccgtacctgcggtataaccacgccgacgccgagtttcaggagcgtctgcaagaagatacgtcatttgggggc |
| | aacctcgggcgagcagtcttccaggccaagaagcgggttctcgaacctctcggtctggttgaggaaggcgctaagacggctcctgcaaagaagagaccggtagagccgtcacc |
| | tcagcgttcccccgactcctccacgggcatcggcaagaaaggccagcagcccgccagaaagagactcaatttcggtcagactggcgactcagagtcagtccccgaccctcaac |
| | ctctcggagaacctccagcagcgccctctagtgtgggatctggtacagtggctgcaggcggtggcgcaccaatggcagacaataacgaaggtgccgacggagtgggtaatgcc |
| | tcaggaaattggcattgcgattccacatggctgggcgacagagtcattaccaccagcacccgaacctgggccctgcccacctacaacaaccacctctacaagcaaatctccagtg |
| | aaactgcaggtagtaccaacgacaacacctacttcggctacagcaccccctgggggtattttgactttaacagattccactgccacttctcaccacgtgactggcagcgactcatca |
| | acaacaactggggattccggcccaagaagctgcggttcaagctcttcaacatccaggtcaaggaggtcacgacgaatgacggcgttacgaccatcgctaataaccttaccagcac |
| | gattcaggtattctcggactcggaataccagctgccgtacgtcctcggctctgcgcaccagggctgcctgcctccgttcccggcggacgtcttcatgattcctcagtacggctacct |
| | gactctcaacaatggcagtcagtctgtgggacgttcctccttctactgcctggagtacttccccttcagatgctgagaacgggcaacaactttgagttcagctacagcttcgaggac |
| | gtgcctttccacagcagctacgcacacagccagagcctggaccggctgatgaatcccctcatcgaccagtacttgtactacctggccagaacacagagtaacccaggaggcaca |
| | gctggcaatcgggaactgcagttttaccaggggggccttcaactatggccgaacaagccaagaattggttacctggaccttgcttccggcaacaaagagtctccaaaacgctgg |
| | atcaaaacaacaacagcaactttgcttggactggtgccaccaaatatcacctgaacggcagaaactcgttggttaatcccggcgtcgccatggcaactcacaaggacgacgagga |
| | ccgctttttcccatccagcggagtcctgatttttggaaaaactggagcaactaacaaaactacattggaaaatgtgttaatgacaaatgaagaagaaattcgtcctactaatcctgtagc |
| | cacggaagaatacgggatagtcagcagcaacttacaagcggctaatactgcagcccagacacaagttgtcaacaaccagggagccttacctggcatggtctggcagaaccggg |
| | acgtgtacctgcagggtcccatctgggccaagattcctcacacggatggcaactttcacccgtctcctttgatgggcggctttggacttaaacatccgcctcctcagatcctgatcaa |
| | gaacactcccgttcccgctaatcctccggaggtgtttactcctgccaagtttgcttcgttcatcacacagtacagcaccggacaagtcagcgtggaaatcgagtgggagctgcagaa |
| | ggaaaacagcaaggctggaacccggagattcagtacacctccaactttgaaaagcagactggtgtggactttgccgttgacagccagggtgtttactctgagcctcgccctattg |
| | gcactcgttacctcacccgtaatctgtaa |
| 23 | atggctgccgatggttatcttccagattggctcgaggacaacctctctgagggcattcgcgagtggtgggcgctgaaacNNNgagccccgaagcccaaagccaaccagcaaa |
| | agcaggacgacggccggggtctggtgcttcctggctacaagtacctcggacccttaacggactcgacaagggggagcccgtcaacgcggcggacgcagcggccctcgagc |
| | acgacaaggcctacgaccagcagctgcaggcgggtgacaatccgtacctgcggtataaccacgccgacgccgagtttcaggagcgtctgcaagaagatacgtcttttgggggc |
| | aacctcgggcgagcagtcttccaggccaagaagcgggttctcgaacctctcggtctggttgaggaaggcgctaagacggctcctggaaagaagagaccggtagagccatcacc |
| | ccagcgttctccagactccttctacgggcatcggcaagaaaggccaacagcccgccagaaaaagactcaattttggtcagactggcgactcagagtcagttccagaccctcaacct |
| | ctcggagaacctccagcagcgccctctggtgtgggacctaatacaatggctgcaggcggtggcgcaccaatggcagacaataacgaaggcgccgacggaggggtagttcctc |
| | gggaaattggcattgcgattccacatggctgggcgacagagtcatcaccaccagcacccgaacctgggccctgcccacctacaacaaccacctctacaagcaaatctccaacgg |
| | gacatcgggaggagccaccaacgacaacacctacttcggctacagcaccccctgggggtattttgactttaacagattccactgccacttttcaccacgtgactggcagcgactcat |
| | caacaacaactggggattccggcccaagagactcagcttcaagctcttcaacatccaggtcaaggaggtcacgcagaatgaaggcaccaagaccatcgccaataacctcaccag |
| | caccatccaggtgtttacggactcggagtaccagctgccgtacgttctcggctctgcccaccagggctgcctgcctccgttcccggcggacgtgttcatgattccccagtacggcta |
| | cctaacactcaacaacggtagtcaggccgtgggacgctcctccttctactgcctggaatactttccttcgcagatgctgagaaccggcaacaacttccagtttacttacaccttcgag |
| | gacgtgcctttccacagcagctacgcccacagccagagcttggaccggctgatgaatcctctgattgaccagtacctgtactacttgtctcggactcaaacaacaggaggcacggc |
| | aaatacgcagactctgggcttcagccaaggtgggcctaatacaatggccaatcaggcaaagaactggctgccaggaccctgttaccgccaacaacgcgtctcaacgacaaccg |
| | ggcaaaacaacaatagcaactttgcctggactgctgggaccaaataccatctgaatggaagaaattcattggctaatcctggcatcgctatggcaacacacaaagacgacgagga |
| | gcgtttttttcccagtaacgggatcctgatttttggcaaacaaaatgctgccagagacaatgcggattacagcgatgtcatgctcaccagcgaggaagaaatcaaaaccactaaccc |
| | tgtggctacagaggaatacggtatcgtggcagataacttgcagcagcaaaacacggctcctcaaattggaactgtcaacagccagggggccttacccggtatggtctggcagaac |
| | cgggacgtgtacctgcagggtcccatctgggccaagattcctcacacggacggcaaccttccacccgtctccgctgatgggcggctttggcctgaaacatcctccgcctcagatcct |
| | gatcaagaacacgcctgtacctgcggatcctccgaccaccttcaaccagtcaaagctgaactctttcatcacgcaatacagcaccggacaggtcagcgtggaaattgaatgggag |
| | ctgcagaaggaaaacagcaagcgctggaaccccgagatccagtacacctccaactactacaaatctacaagtgtggactttgctgttaatacagaaggcgtgtactctgaaccccg |
| | ccccattggcacccgttacctcacccgtaatctgtaa |
| 24 | atggctgccgatggttatcttccagattggctcgaggacaaccttagtgaaggaattcgcgagtggtgggctttgaaacNNNgagcccctcaacccaaggcaaatcaacaacat |
| | caagacaacgctcgaggtcttgtgcttccgggttacaaataccttggacccggcaacggactcgacaagggggagccggtcaacgcagcagacgcggcggccctcgagcacg |
| | acaaggcctacgaccagcagctcaaggccggagacaacccgtacctcaagtacaaccacgccgacgccgagttccaggagcggctcaaagaagatacgtcttttgggggcaa |
| | cctcgggcgagcagtcttccaggccaaaaagaggcttcttgaacctcttggtctggttgaggaagcggctaagacggctcctggaaagaagaggcctgtagagcagtctcctcag |
| | gaaccggactcctccgcgggtattggcaaatcgggtgcacagcccgctaaaaagagactcaatttcggtcagactggcgacacagagtcagtcccagaccctcaaccaatcgga |
| | gaacctcccgcagccccctcaggtgtgggatctcttacaatggcttcaggtggtggcgcaccagtggcagacaataacgaaggtgccgatggagtgggtagttcctcgggaaatt |
| | ggcattgcgattcccaatggctgggggacagagtcatcaccaccagcacccgaacctgggccctgcccacctacaacaatcacctctacaagcaaatctccaacagcacatctg |
| | gaggatcttcaaatgacaacgcctacttcggctacagcaccccctgggggtattttgacttcaacagattccactgccacttctcaccacgtgactggcagcgactcatcaacaaca |
| | actggggattccggcctaagcgactcaacttcaagctcttcaacattcaggtcaaagaggttacggacaacaatggagtcaagaccatcgccaataaccttaccagcacggtccag |
| | gtcttcacggactcagactatcagctcccgtacgtgctcgggtcggctcacgagggctgcctcccgccgttcccagcggacgttttcatgattcctcagtacgggtatctgacgctta |
| | atgatggaagccaggccgtgggtcgttcgtccttttactgcctggaatatttcccgtcgcaaatgctaagaacgggtaacaacttccagttcagctacgagtttgagaacgtacctttc |
| | catagcagctacgctcacagccaaagcctggaccgactaatgaatccactcatcgaccaatacttgtactatctctcaaagactattaacggttctggacagaatcaacaaacgctaa |
| | aattcagtgtggccggacccagcaacatggctgtccagggaagaaactacatacctggacccagctaccgacaacaacgtgtctcaaccactgtgactcaaaacaacaacagcg |
| | aatttgcttggcctggagcttcttcttgggctctcaatggacgtaatagcttgatgaatcctggacctgctatggccagccacaaagaaggagaggaccgtttctttcctttgtctggat |
| | ctttaatttttggcaaacaaggaactggaagagacaacgtggatgcggacaaagtcatgataaccaacgaagaagaaattaaaactactaacccggtagcaacggagtcctatgg |
| | acaagtggccacaaaccaccagagtgcccaagcacaggcgcagaccggctgggttcaaaaccaaggaatacttccgggtatggtttggcaggacagagatgtgtacctgcaag |
| | gacccatttgggccaaaattcctcacacggacggcaactttcacccttctccgctgatgggagggtttggaatgaagcacccgcctcctcagatcctcatcaaaaacacacctgtac |
| | ctgcggatcctccaacggccttcaacaaggacaagctgaactctttcatcacccagtattctactggccaagtcagcgtggagatcgagtgggagctgcagaaggaaaacagcaa |
| | gcgctggaacccggagatccagtacacttccaactattacaagtctaataatgttgaatttgctgttaatactgaaggtgtatatagtgaaccccgccccattggcaccagatacctga |
| | ctcgtaatctgtaa |
| 25 | atggctgctgacggttatcttccagattggctcgaggacaacctctctgagggcattcgcgaggggggacctgaaacNNNgagcccccaagcccaaggccaaccagcaga |
| | agcaggacgacggccggggtctggtgcttcctggctacaagtacctcggacccttcaacggactcgacaagggggagcccgtcaacgcggcggacgcagcggccctcgagc |
| | acgacaaggcctacgaccagcagctcaaagcgggtgacaatccgtacctgcggtataaccacgccgacgccgagtttcaggagcgtctgcaagaagatacgtcttttgggggc |
| | aacctcgggcgagcagtcttccaggccaagaagcgggttctcgaacctctcggtctggttgaggaagctgctaagacggctcctggaaagaagagaccggtagaaccgtcacct |
| | cagcgttcccccgactcctccacgggcatcggcaagaaaggccagcagcccgctaaaaagagactgaactttgggcagactggcgagtcagagtcagtccccgaccctcaac |
| | caatcggagaaccaccagcaggcccctctggtctgggatctggtacaatggctgcaggcggtggcgctccaatggcagacaataacgaaggcgccgacggagtgggtagttc |
| | ctcaggaaattggcattgcgattccacatggctgggcgacagagtcatcaccaccagcacccgaacctgggccctgcccacctacaacaaccacctctacaagcaaatctccaac |
| | gggacatcgggaggaagcaccaacgacaacacctacttcggctacagcaccccctgggggtattttgacttcaacagattccactgccacttctcaccacgtgactggcagcgac |
| | tcatcaacaacaactggggattccggccaaaaagactcagcttcaagctcttcaacatccaggtcaaggaggtcacgcagaatgaaggcaccaagaccatcgccaataaccttac |
| | cagcacgattcaggtatttacggactcggaataccagctgccgtacgtcctcggctccgcgcaccagggctgcctgcctccgttcccggcggatgtcttcatgattccccagtacg |
| | gctacctgacactgaacaatggaagtcaagccgtaggccgttcctccttctactgcctggaatattttccatctcaaatgctgcgaactggaaacaattttgaattcagctacaccttcg |
| | aggacgtgcctttccacagcagctacgcacacagccagagcttggaccgactgatgaatcctctcattgaccagtacctgtactacttatccagaactcagtccacaggaggaact |
| | caaggtacccagcaattgttattttctcaagctgggcctgcaaacatgtcggctcaggccaagaactggctgcctggaccttgctaccggcagcagcgagtctccacgacactgtc |
| | gcaaaacaacaacagcaactttgcttggactggtgccaccaaatatcacctgaacggaagagactctctggtgaatcccggtgtcgccatggcaacccacaaggacgacgagga |
| | acgcttcttcccgtcgagcggagtcctgatgtttggaaaacagggtgctggaagagacaatgtggactacagcagcgttatgctaacaagcgaagaagaaattaaaaccactaac |
| | cctgtagccacagaacaatacggcgtggtggctgacaacttgcagcaagccaatacagggcctattgtgggaaatgtcaacagccaaggagccttacctggcatggtctggcag |
| | aaccgagacgtgtacctgcagggtcccatctgggccaagattcctcacacggacggcaactttcacccgtctcctctgatgggcggctttggacttaaacacccgcctccacagat |
| | cctgatcaagaacacgccggtacctgcggatcctccaacaacgttcagccaggcgaaattggcttccttcatcacgcagtacagcaccggacaggtcagcgtggaaatcgagtg |
| | ggagctgcagaaggagaacagcaaacgctggaacccagagattcagtacacttcaaactactacaaatctacaaatgtggactttgctgtcaatacagagggaacttattctgagc |
| | ctcgccccattggtactcgttatctgacacgtaatctgtaa |
| 26 | atggctgctgacggttatcttccagattggctcgaggacaacctctctgagggcattcgcgagtggtgggacctgaaacNNNgagccccgaagcccaaggccaaccagcaga |
| | agcaggacgacggccggggtctggtgcttcctggctacaagtacctcggacccttcaacggactcgacaagggggagcccgtcaacgcggcggacgcagcggccctcgagc |
| | acgacaaggcctacgaccagcagctcaaagcgggtgacaatccgtacctgcggtataaccacgccgacgccgagtttcaggagcgtctgcaagaagatacgtcttttgggggc |
| | aacctcgggcgagcagtcttccaggccaagaagagggtactcgaacctctgggcctggttgaagaaggtgctaaaacggctcctggaaagaagagaccgttagagtcaccaca |
| | agagcccgactcctcctcgggcatcggcaaaaaaggcaaacaaccagccagaaagaggctcaactttgaagaggacactggagccggagacggaccccctgaaggatcaga |
| | taccagcgccatgtcttcagacattgaaatgcgtgcagcaccgggcggaaatgctgtcgatgcgggacaaggttccgatggagtgggtaatgcctcgggtgattggcattgcgatt |
| | ccacctggtctgagggcaaggtcacaacaacctcgaccagaacctgggtcttgcccacctacaacaaccacttgtacctgcgtctcggaacaacatcaagcagcaacacctacaa |
| | cggattctccaccccctggggatattttgacttcaacagattccactgtcacttctcaccacgtgactggcaaagactcatcaacaacaactggggactacgaccaaaagccatgcg |
| | cgttaaaatcttcaatatccaagttaaggaggtcacaacgtcgaacggcgagactacggtcgctaataaccttaccagcacggttcagatatttgcggactcgtcgtatgagctcccg |
| | tacgtgatggacgctggacaagaggggagcctgcctcctttccccaatgacgtgttcatggtgcctcaatatggctactgtggcatcgtgactggcgagaatcagaaccaaacgga |
| | cagaaacgctttctactgcctggagtattttccttcgcaaatgttgagaactggcaacaactttgaaatggcttacaactttgagaaggtgccgttccactcaatgtatgctcacagcca |
| | gagcctggacagactgatgaatcccctcctggaccagtacctgtggcacttacagtcgactacctctggagagactctgaatcaaggcaatgcagcaaccacatttggaaaaatca |
| | ggagtggagactttgccttttacagaaagaactggctgcctgggccttgtgttaaacagcagagattctcaaaaactgccagtcaaaattacaagattcctgccagcgggggcaac |
| | gctctgttaaagtatgacacccactataccttaaacaaccgctggagcaacatcgcgcccggacctccaatggccacagccggaccttcggatggggacttcagtaacgcccagc |
| | ttatattccctggaccatctgttaccggaaatacaacaacttcagccaacaatctgttgtttacatcagaagaagaaattgctgccaccaacccaagagacacggacatgtttggcca |
| | gattgctgacaataatcagaatgctacaactgctcccataaccggcaacgtgactgctatgggagtgctgcctggcatggtgtggcaaaacagagacatttactaccaagggccaa |
| | tttgggccaagatcccacacgcggacggacattttcatccttcaccgctgattggtgggtttggactgaaacacccgcctccccagatattcatcaagaacactcccgtacctgccaa |
| | tcctgcgacaaccttcactgcagccagagtggactctttcatcacacaatacagcaccggccaggtcgctgttcagattgaatgggaaattgaaaaggaacgctccaaacgctgga |
| | atcctgaagtgcagtttacttcaaactatgggaaccagtcttctatgttgtgggctcctgatacaactgggaagtatacagagccgcgggttattggctctcgttatttgactaatcatttg |
| | taa |
| 27 | atggctgctgacggttatcttccagattggctcgaggacaacctctctgaaggcattcgcgagtggtgggcgctgaaacNNNgagctccacaacccaaggccaaccaacagc |
| | atcaggacaacggcaggggtcttgtgcttcctgggtacaagtacctcggacccttcaacggactcgacaagggagagccggtcaacgaggcagacgccgcggccctcgagca |
| | cgacaaggcctacgacaagcagctcgagcagggggacaacccgtatctcaagtacaaccacgccgacgccgagttccagcagcgcttggcgaccgacacctcttttgggggc |
| | aacctcgggcgagcagtcttccaggccaaaaagaggattctcgagcctctgggtctggttgaagagggcgttaaaacggctcctggaaagaaacgcccattagaaaagactcca |
| | aatcggccgaccaacccggactctgggaaggccccggccaagaaaaagcaaaaagacggcgaaccagccgactctgctagaaggacactcgactttgaagactctggagca |
| | ggagacggaccccctgagggatcatcttccggagaaatgtctcatgatgctgagatgcgtgcggcgccaggcggaaatgctgtcgaggcgggacaaggtgccgatggagtgg |
| | gtaatgcctccggtgattggcattgcgattccacctggtcagagggccgagtcaccaccaccagcacccgaacctgggtcctacccacgtacaacaaccacctgtacctgcgaat |
| | cggaacaacggccaacagcaacacctacaacggattctccaccccctggggatactttgactttaaccgcttccactgccacttttccccacgcgactggcagcgactcatcaaca |
| | acaactggggactcaggccgaaatcgatgcgtgttaaaatcttcaacatacaggtcaaggaggtcacgacgtcaaacggcgagactacggtcgctaataaccttaccagcacggt |
| | tcagatctttgcggattcgacgtatgaactcccatacgtgatggacgccggtcaggaggggagctttcctccgtttcccaacgacgtctttatggttccccaatacggatactgcgga |
| | gttgtcactggaaaaaaccagaaccagacagacagaaatgccttttactgcctggaatactttccatcccaaatgctaagaactggcaacaattttgaagtcagttaccaatttgaaaa |
| | agttcctttccattcaatgtacgcgcacagccagagcctggacagaatgatgaatcctttactggatcagtacctgtggcatctgcaatcgaccactaccggaaattcccttaatcaag |
| | gaacagctaccaccacgtacgggaaaattaccactggagactttgcctactacaggaaaaactggttgcctggagcctgcattaaacaacaaaaattttcaaagaatgccaatcaaa |
| | actacaagattcccgccagcgggggagacgcccttttaaagtatgacacgcataccactctaaatgggcgatggagtaacatggctcctggacctccaatggcaaccgcaggtgc |
| | cggggactcggattttagcaacagccagctgatctttgccggacccaatccgagcggtaacacgaccacatcttcaaacaatttgttgtttacctcagaagaggagattgccacaac |
| | aaacccacgagacacggacatgtttggacagattgcagataataatcaaaatgccaccaccgcccctcacatcgctaacctggacgctatgggaattgttcccggaatggtctggc |
| | aaaacagagacatctactaccagggccctattgggccaaggtccctcacacggacggacactttcacccttcgccgctgatgggaggatttggactgaaacacccgcctccaca |
| | gattttcatcaaaaacacccccgtacccgccaatcccaatactacctttagcgctgcaaggattaattcttttctgacgcagtacagcaccggacaagttgccgttcagatcgactggg |
| | aaattcagaaggagcattccaaacgctggaatcccgaagttcaatttacttcaaactacggcactcaaaattctatgctgtgggctcccgacaatgctggcaactaccacgaactcc |
| | gggctattgggtcccgtttcctcacccaccacttgtaa |
| 28 | atgactgacggttaccttccagattggctagaggacaacctctctgaaggcgttcgagagtggtgggcgctgcaacNNNgagcccctaaacccaaggcaaatcaacaacatca |
| | ggacaacgctcggggtcttgtgcttccgggttacaaatacctcggacccggcaacggacttgacaagggggaacccgtcaacgcagcggacgcggcagccctcgaacacgac |
| | aaggcctacgaccagcagctcaaggccggtgacaacccctacctcaagtacaaccacgccgacgccgagtttcaggagcgtcttcaagaagatacgtcttttgggggcaacctc |
| | ggacgagcagtcttccaggccaaaaagaggatccttgagcctctgggtctggttgaggaagcggctaagacggctcctggaaaaaagagacctgtagagcaatctccagcaga |
| | accggactcctcttcgggcatcggcaaatcaggccagcagcccgctagaaaaagactgaattttggtcagactggcgacacagagtcagtcccagaccctcaaccactcggaca |
| | acctcccgcagccccctctggtgtgggatctactacaatggcttcaggcggtggcgcaccaatggcagacaataacgagggtgccgatggagtgggtaattcctcaggaaattgg |
| | cattgcgattcccaatggctgggcgacagagtcatcaccaccagcacccgcacctgggccctgcccacctacaacaatcacctctacaagcaaatctccagccaatcaggagcc |
| | accaacgacaaccactactttggctacagcaccccctgggggtattttgacttcaacagattccactgccacttttcaccacgtgactggcaaagactcatcaacaacaactggggat |
| | tccgacccaagagactcaacttcaagctctttaacattcaagtcaaagaggtcacgcagaatgacggtacgacgacgattgccaataaccttaccagcacggttcaggtgtttactg |
| | actccgagtaccagctcccgtacgtcctcggctcggcgcatcagggatgcctcccgccgttcccagcagacgtcttcatggtcccacagtatggatacctcaccctgaacaacgg |
| | gagtcaggcggtaggacgctcttccttttactgcctggagtactttccttctcagatgctgcgtactggaaacaactttcagtttagctacacttttgaagacgtgcctttccacagcagc |
| | tacgctcacagccaaagtctggaccgtctcatgaatcctctgatcgaccagtacctgtactatctgaacaggacacaaacagccagtggaactcagcagtctcggctactgtttagc |
| | caagctggacccaccagtatgtctcttcaagctaaaaactggctgcctggaccttgctacagacagcagcgtctgtcaaagcaggcaaacgacaacaacaacagcaactttccctg |
| | gactggtgccaccaaatatcatctgaatggccgggactcattggtgaacccgggccctgctatggccagtcacaaggatgacaaagaaaagtttttccccatgcatggaaccctga |
| | tatttggtaaagaaggaacaaatgccaacaacgcggatttggaaaatgtcatgattacagatgaagaagaaatccgcaccaccaatcccgtggctacggagcagtacgggactgt |
| | gtcaaataatttgcaaaactcaaacgctggtccaactactggaactgtcaatcaccaaggagcgttacctggtatggtgtggcaggatcgagacgtgtacctgcagggacccatttg |
| | ggccaagattcctcacaccgatggacactttcatccttctccactgatgggaggttttgggctcaaacacccgcctcctcagatcatgatcaaaaacactcccgttccagccaatcct |
| | cccacaaactttagtgcggcaaagtttgcttccttcatcacacagtactccacggggcaggtcagcgtggagatcgagtgggagctgcagaaggagaacagcaaacgctggaat |
| | cccgaaattcagtacacttccaactacaacaaatctgttaatgtggactttactgtggacactaatggtgtgtattcagagcctcgccccattggcaccagatacctgactcgtaatctg |
| | taa |
| 29 | atggctgccgatggttatcttccagattggctcgaggacaacctctctgagggcattcgcgagtggtgggacttgaaacNNNgagccccgaaacccaaagccaaccagcaaa |
| | agcaggacgacggccggggtctggtgcttcctggctacaagtacctcggacccttcaacggactcgacaagggggagcccgtcaacgcggcggacgcagcggccctcgagc |
| | acgacaaagcctacgaccagcagctcaaagcgggtgacaatccgtacctgcggtataatcacgccgacgccgagtttcaggagcgtctgcaagaagatacgtcttttgggggca |
| | acctcgggcgagcagtcttccaggccaagaagcgggttctcgaacctctcggtctggttgaggaaggcgctaagacggctcctggaaagaagagaccggtagagcagtcgcc |
| | acaagagccagactcctcctcgggcatcggcaagacaggccagcagcccgctaaaaagagactcaattttggtcagactggcgactcagagtcagtccccgacccacaacctct |
| | cggagaacctccagcagccccctcaggtctgggacctaatacaatggcttcaggcggtggcgctccaatggcagacaataacgaaggcgccgacggagtgggtaattcctcgg |
| | gaaattggcattgcgattccacatggctgggggacagagtcatcaccaccagcacccgaacctgggccctgcccacctacaacaaccacctctacaagcaaatctccaacggca |
| | cctcgggaggaagcaccaacgacaacacctattttggctacagcaccccctgggggtattttgacttcaacagattccactgtcacttttcaccacgtgactggcaacgactcatcaa |
| | caacaattggggattccggcccaaaagactcaacttcaagctgttcaacatccaggtcaaggaagtcacgacgaacgaaggcaccaagaccatcgccaataatctcaccagcac |
| | cgtgcaggtctttacggactcggagtaccagttaccgtacgtgctaggatccgctcaccagggatgtctgcctccgttcccggcggacgtcttcatggttcctcagtacggctattta |
| | actttaaacaatggaagccaagccctgggacgttcctccttctactgtctggagtatttcccatcgcagatgctgagaaccggcaacaactttcagttcagctacaccttcgaggacgt |
| | gcctttccacagcagctacgcgcacagccagagcctggacaggctgatgaatcccctcatcgaccagtacctgtactacctggtcagaacgcaaacgactggaactggagggac |
| | gcagactctggcattcagccaagcgggtcctagctcaatggccaaccaggctagaaattgggtgcccggaccttgctaccggcagcagcgcgtctccacgacaaccaaccaga |
| | acaacaacagcaactttgcctggacgggagctgccaagtttaagctgaacggccgagactctctaatgaatccgggcgtggcaatggcttcccacaaggatgacgacgaccgct |
| | tcttcccttcgagcggggtcctgatttttggcaagcaaggagccgggaacgatggagtggattacagccaagtgctgattacagatgaggaagaaatcaaggctaccaaccccgt |
| | ggccacagaagaatatggagcagtggccatcaacaaccaggccgccaatacgcaggcgcagaccggactcgtgcacaaccagggggtgattcccggcatggtgtggcagaa |
| | tagagacgtgtacctgcagggtcccatctgggccaaaattcctcacacggacggcaactttcacccgtctcccctgatgggcggctttggactgaagcacccgcctcctcaaattct |
| | catcaagaacacaccggttccagcggacccgccgcttaccttcaaccaggccaagctgaactctttcatcacgcagtacagcaccggacaggtcagcgtggaaatcgagtggga |
| | gctgcagaaagaaaacagcaaacgctggaatccagagattcaatacacttccaactactacaaatctacaaatgtggactttgctgtcaacacggagggggtttatagcgagcctc |
| | gccccattggcacccgttacctcacccgcaacctgtaa |
| 30 | atggctgccgatggttatcttccagattggctcgaggacaacctctctgagggcattcgcgagtggtgggacttgaaacNNNgagccccgaaacccaaagccaaccagcaaa |
| | agcaggacgacggccggggtctggtgcttcctggctacaagtacctcggacccttcaacggactcgacaagggggagcccgtcaacgcggcggacgcagcggccctcgagc |
| | acgacaaggcctacgaccagcagctcaaagcgggtgacaatccgtacctgcggtataaccacgccgacgcgagtttcaggagcgtctgcaagaagatacgtcttttgggggc |
| | aacctcgggcgagcagtcttccaggccaagaagcgggttctcgaacctctcggtctggttgaggaaggcgctaagacggctcctggaaagaagagaccggtagagccatcacc |
| | cagcgttctccagactcctctacgggcatcggcaagaaaggccagcagcccgcgaaaaagagactcaactttgggcagactggcgactcagagtcagtgcccgaccctcaac |
| | caatcggagaaccccccgcaggcccctctggtctgggatctggtacaatggctgcaggcggtggcgctccaatggcagacaataacgaaggcgccgacggagtgggtagttc |
| | ctcaggaaattggcattgcgattccacatggctgggcgacagagtcatcaccaccagcacccgaacctgggccctccccacctacaacaaccacctctacaagcaaatctccaac |
| | gggacttcgggaggaagcaccaacgacaacacctacttcggctacagcaccccctgggggtattttgactttaacagattccactgccacttctcaccacgtgactggcagcgact |
| | catcaacaacaactggggattccggcccaagagactcaacttcaagctcttcaacatccaggtcaaggaggtcacgcagaatgaaggcaccaagaccatcgccaataaccttacc |
| | agcacgattcaggtctttacggactcggaataccagctcccgtacgtcctcggctctgcgcaccagggctgcctgcctccgttcccggcggacgtcttcatgattcctcagtacggg |
| | tacctgactctgaacaatggcagtcaggccgtgggccgttcctccttctactgcctggagtactttccttctcaaatgctgagaacgggcaacaactttgagttcagctaccagtttga |
| | ggacgtgccttttcacagcagctacgcgcacagccaaagcctggaccggctgatgaaccccctcatcgaccagtacctgtactacctgtctcggactcagtccacgggaggtacc |
| | gcaggaactcagcagttgctattttctcaggccgggcctaataacatgtcggctcaggccaaaaactggctacccgggccctgctaccggcagcaacgcgtctccacgacactgt |
| | cgcaaaataacaacagcaactttgcctggaccggtgccaccaagtatcatctgaatggcagagactctctggtaaatcccggtgtcgctatggcaacccacaaggacgacgaaga |
| | gcgattttttccgtccagcggagtcttaatgtttgggaaacagggagctggaaaagacaacgtggactatagcagcgttatgctaaccagtgaggaagaaattaaaaccaccaacc |
| | cagtggccacagaacagtacggcgtggtggccgataacctgcaacagcaaaacgccgctcctattgtaggggccgtcaacagtcaaggagccttacctggcatggtctggcag |
| | aaccgggacgtgtacctgcagggtcctatctgggccaagattcctcacacggacggaaactttcatccctcgccgctgatgggaggctttggactgaaacacccgcctcctcagat |
| | cctgattaagaatacacctgttcccgcggatcctccaactaccttcagtcaagctaagctggcgtcgttcatcacgcagtacagcaccggacaggtcagcgtggaaattgaatggg |
| | agctgcagaaagaaaacagcaaacgctggaacccagagattcaatacacttccaactactacaaatctacaaatgtggactttgctgttaacacagatggcacttattctgagcctcg |
| | ccccatcggcacccgttacctcacccgtaatctgtaa |
| 31 | aacatgctacgcagagagggagtgg |
| 32 | catgagacaaggaacccctagtgatggag |
| 33 | MKRNYILGLDIGITSVGYGIIDYETRDVIDAGVRLFKEANVENNEGRRSKRGARRLKRRRRHRIQRVKKLLFDYN |
| | LLTDHSELSGINPYEARVKGLSQKLSEEEFSAALLHLAKRRGVHNVNEVEEDTGNELSTKEQISRNSKALEEKYV |
| | AELQLERLKKDGEVRGSINRFKTSDYVKEAKQLLKVQKAYHQLDQSFIDTYIDLLETRRTYYEGPGEGSPFGWK |
| | DIKEWYEMLMGHCTYFPEELRSVKYAYNADLYNALNDLNNLVITRDENEKLEYYEKFQIIENVFKQKKKPTLKQ |
| | IAKEILVNEEDIKGYRVTSTGKPEFTNLKVYHDIKDITARKEIIENAELLDQIAKILTIYQSSEDIQEELTNLNSELTQ |
| | EEIEQISNLKGYTGTHNLSLKAINLILDELWHTNDNQIAIFNRLKLVPKKVDLSQQKEIPTTLVDDFILSPVVKRSFI |
| | QSIKVINAIIKKYGLPNDIHIELAREKNSKDAQKMINEMQKRNRQTNERIEEIIRTTGKENAKYLIEKIKLHDMQEGK |
| | CLYSLEAIPLEDLLNNPFNYEVDHIIPRSVSFDNSFNNKVLVKQEENSKKGNRTPFQYLSSSDSKISYETFKKHILNL |
| | AKGKGRISKTKKEYLLEERDINRFSVQKDFINRNLVDTRYATRGLMNLLRSYFRVNNLDVKVKSINGGFTSFLRR |
| | KWKFKKERNKGYKHHAEDALIIANADFIFKEWKKLDKAKKVMENQMFEEKQAESMPBIETEQEYKEIFITPHQIK |
| | HIKDFKDYKYSHRVDKKPNRELINDTLYSTRKDDKGNTLIVNNLNGLYDKDNDKLKKLINKSPEKLLMYHHDPQ |
| | TYQKLKLIMEQYGDEKNPLYKYYEETGNYLTKYSKKDNGPVIKKIKYYGNKLNAHLDITDDYPNSRNKVVKLSL |
| | KPYRFDVYLDNGVYKFVTVKNLDVIKKENYYEVNSKCYEEAKKLKKISNQAEFIASFYNNDLIKINGELYRVIGV |
| | NNDLLNRIEVNMIDITYREYLENMNDKRPPRIIKTIASKTQSIKKYSTDILGNLYEVKSKKHPQIIKKG |
| 34 | atgaaaaggaattatatcttaggattagatatcggaattacatcagtgggttatggaattattgattatgaaactagagatgtcatagatgcgggcgtacgtttatttaaagaggctaatgt |
| | tgaaaataatgaaggacgacgatcaaaaagaggtgccagaaggcttaagaggcgtcgtagacatagaatacaaagagtaaagaaacttttatttgattacaatttgttgacagatcat |
| | agtgagctaagtggaatcaatccttacgaggcgcgcgtaaagggattaagtcaaaaattaagtgaagaggaattttctgcggcattgctacatttagcaaagcgtagaggtgtacat |
| | aatgttaatgaagtggaagaagatacaggtaatgaattatccactaaagaacaaatttcaagaaatagtaaagcgttagaagagaagtatgttgcagaattacagttggaacgtttga |
| | aaaaagacggtgaagtgagaggttcgattaaccgtttcaaaacatctgactatgtaaaagaagcaaagcagttattaaaagtacaaaaagcatatcatcaacttgatcaatcatttata |
| | gacacttatattgatttattggaaacaagaagaacatattatgagggaccaggtgaaggtagcccatttggatggaaagatattaaagaatggtatgaaatgttaatgggacattgtac |
| | gtatttcccagaagaattacgtagtgtgaaatatgcctataatgctgatttatataatgcgctgaatgatttgaacaacttggttattacacgagatgagaatgagaagctagagtattatg |
| | aaaaattccaaattatcgagaatgtctttaaacaaaagaaaaagccgacgcttaaacaaattgcgaaggaaatcttggtgaatgaagaagacatcaaaggctatcgtgtcacaagta |
| | caggtaaaccagaatttacaaacttgaaagtttatcacgatatcaaagatattacagcaagaaaagaaattatcgagaatgcagagctactcgatcaaatagctaaaatattaactattt |
| | accagtcatcagaagatatacaagaagaattaacaaacctaaattcagaattgacacaagaagagattgaacaaatttcaaacttgaaaggttatacaggaactcataacctttcacta |
| | aaggcaataaatttaatattagacgaattgtggcatacgaacgataatcaaatagctattttcaatcgtttgaaacttgtacctaaaaaggtagatttaagccaacaaaaagasattccta |
| | ctactttagttgatgattttatactgtctccagtagtgaaacgttcatttatacaatctattaaagttattaacgctattattaaaaaatacggtttgccaaatgatattattattgaacttgcgag |
| | agaaaagaattctaaagatgcacaaaaaatgattaatgaaatgcagaagagaaatcgtcaaacgaatgaacgtattgaggaaattataagaacgacaggtaaagaaaatgctaaat |
| | atttaattgaaaaaattaagctgcacgatatgcaagaagggaaatgtttatactcgttagaagcaatccctctagaagatttacttaataatccattcaattacgaagtagaccatatcatt |
| | ccacgttctgtttctttcgataactctttcaataataaagtgttggtgaaacaagaagaaaatagtaaaaaaggtaatagaacgccatttcaatatttaagttcttcagattctaaaataagtt |
| | atgagacattcaaaaagcatattttaaatcttgctaaaggcaaaggtagaatctctaagacgaaaaaagaatatttgttagaagaacgagatatcaatcgcttcagtgtccaaaaagat |
| | tttattaaccgtaacttagtagatacacgctatgcgacaagagggttaatgaacttattaagatcttattttagagtgaataacttagatgtcaaagtgaaatcgattaatggcggattcac |
| | aagtttcttaagaaggaaatggaagttcaaaaaagaaagaaataagggctacaaacaccatgctgaagatgcactgattattgcgaacgctgattttattttcaaagaatggaaaaaa |
| | ctagataaagctaaaaaagtgatggaaaatcaaatgtttgaagaaaagcaagctgaaagtatgcctgaaattgagactgagcaagagtataaagaaatttttataacgcctcatcaaa |
| | ttaaacatattaaggattttaaagattataaatattcacatagagttgataaaaagccgaatagagagttaataaatgatacattatattctacgagaaaagatgacaagggtaatacatta |
| | atcgttaataacttaaatggtttatacgataaagataatgataaattgaaaaaattaattaataaatcacctgaaaaattattgatgtatcatcatgatccacaaacatatcaaaaattaaaat |
| | tgatcatggaacaatatggcgatgagaaaaatccgctttataaatattatgaagaaacaggcaattacttaacaaaatatagtaaaaaagataacggaccagtcatcaaaaaaattaa |
| | atattatggtaacaagctaaatgcgcatttagatattacggatgattatccaaatagcagaaataaagtagtaaaactttcattaaaaccatatcgctttgatgtttatttagataatggggt |
| | atataaatttgtgacagttaaaaatttagatgttatcaaaaaagaaaactactatgaagttaattcaaagtgttatgaagaagcaaaaaaactgaagaaaattagtaatcaagcagaattt |
| | atcgcaagtttttacaataatgacttgattaagattaacggagaattatatagagtcataggtgtaaataatgatctacttaacagaattgaagtaaatatgatagacatcacatatagaga |
| | atatttagagaacatgaatgataaaagaccacctagaataattaaaacaatagcaagcaaaacacaatctattaaaaagtattctacagatattctaggcaatctttatgaagtgaagag |
| | taaaaagcatcctcaaatcataaagaaaggatga |
| 35 | MDKKYSIGLDIGTNSVGWAVITDDYKVPSKKFKVLGNTDRHSIKKNLIGALLFDSGETAEATRLKRTARRRYTRR |
| | KNRICYLQEIFSNEMAKVDDSFFHRLEESFLVEEDKKHERHPIFGNIVDEVAYHEKYPTIYHLRKKLVDSTDKADL |
| | RLIYLALAHMIKFRGHFLIEGDLNPDNSDVDKLFIQLVQTYNQLFEENPINASGVDAKAILSARLSKSRRLENLIAQ |
| | LPGEKKNGLFGNLIALSLGLTPNFKSNFDLAEDAKLQLSKDTYDDDLDNLLAQIGDQYADLFLAAKNLSDAILLS |
| | DILRLNSEITKAPLSASMIKRYDEHHQDLTLLKALVRQQLPEKYKEIFFDQSKNGYAGYIDGGASQEEFYKFIKPIL |
| | EKMDGTEELLAKLNREDLLRKQRTFDNGSIPHQIHLGELHAILRRQEDFYPFLKDNREKIEKILTFRIPYYVGPLAR |
| | GNSRFAWMTRKSEETITPWNFEEVVDKGASAQSFIERMTNFDKNLPNEKVLPKHSLLYEYFTVYNELTKVKYVT |
| | EGMRKPAFLSGEQKKAIVDLLFKTNRKVTVKQLKEDYFKKIECFDSVEISGVEDRFNASLGTYHDLLKIIKDKDFL |
| | DNEENEDILEDIVLTLTLFEDREMIEERLKTYAHLFDDKVMKQLKRRRYTGWGRLSRKLINGIRDKQSGKTILDFL |
| | KSDGFANRNFMQLIHDDSLTFKEDIQKAQVSGQGDSLHEHIANLAGSPAIKKGILQTVKVVDELVKVMGRHKPE |
| | NIVIEMARENQTTQKGQKNSRERMKRIEEGIKELGSQILKEHPVENTQLQNEKLYLYYLQNGRDMYVDQELDINR |
| | LSDYDVDHIVPQSFIKDDSIDNKVLTRSDKNRGKSDNVPSEEVVKKMKNYWRQLLNAKLITQRKFDNLTKAERG |
| | GLSELDKAGFIKRQLVETRQITKHVAQILDSRMNTKYDENDKLIREVKVITLKSKLVSDFRKDFQFYKVREINNYH |
| | HAHDAYLNAVVGTALIKKYPKLESEFVYGDYKVYDVRKMIAKSEQEIGKATAKYFFYSNIMNFFKTEITLANGEI |
| | RKRPLIETNGETGEIVWDKGRDFATVRKVLSMPQVNIVKKTEVQTGGFSKESILPKRNSDKLIARKKDWDPKKYG |
| | GFDSPTVAYSVLVVAKVEKGKSKKLKSVKELLGLTIMERSSFEKNPIDFLEAKGYKEVKKDLIIKLPKYSLFELEN |
| | GRKRMLASAGELQKGNELALPSKYVNFLYLASHYEKLKGSPEDNEQKQLFVEQHKHYLDEIIEQISEFSKRVILA |
| | DANLDKVLSAYNKHRDKPIREQAENIIHLFTLTNLGAPAAFKYFDTTIDRKRYTSTKEVLDATLIHQSITGLYETRI |
| | DLSQLGGD |
| 36 | LTSTSDPSTDSTRGSPSTRRTQRPSSTTRPTTSSCRRVTIRTCGITTPTPSFRSVCKKIRLLGATSGEQSSRPRSGFSNL |
| | SVWLRKALRRLLERRDR |
| 37 | LEPRSPKPTSKSRTTAGVWCFLAFRSVCKKIRLLGATSGEQSSRPRSGFSNLSVWLRKALRRLLERRDR |
| 38 | LFRSVCKKIRLLGATSGEQSSRPRSGFSNLSVWLRKALRRLLERRDR |
| 39 | LEPRSPKPTSKSRTTAGVWCFLATSTSDPSTDSTRGSPSTRRTQRPSSTTRPTTSSCRRVTIRTCGITTPTPS |
| 40 | LEPRSPKPTSKSRTTATSTSDPSTDSTRGSPSTRRTQRPSSTTRPTTSSCRRVTIRTCGITTPTPSFRSVCKKIRLLGAT |
| | SGEQSSRPRSGFSNLSVWLRKALRRLLERRDR |
| 41 | LEPRSPKPTSKSRTTAGVWCFLARRTQRPSSTTRPTTSSCRRVTIRTCGITTPTPSFRSVCKKIRLLGATSGEQSSRPR |
| | SGFSNLSVWLRKALRRLLERRDR |
| 42 | LEPRSPKPTSKSRTTAGVWCFLATSTSDPSTDSTRGSPSTRRTQRPRRVTIRTCGITTPTPSFRSVCKKIRLLGATSG |
| | EQSSRPRSGFSNLSVWLRKALRRLLERRDR |
| 43 | LEPRSPKPTSKSRTTAGVWCFLATSTSDPSTDSTRGSPSTRRTQRPSSTTRPTTSSCRRVVCKKIRLLGATSGEQSSR |
| | PRSGFSNLSVWLRKALRRLLERRDR |
| 44 | LEPRSPKPTSKSRTTAGVWCFLATSTSDPSTDSTRGSPSTRRTQRPSSTTRPTTSSCRRVTIRTCGITTPTPSFRSVCK |
| | KIRLLGARPRSGFSNLSVWLRKALRRLLERRDR |
| 45 | LEPRSPKPTSKSRTTAGVWCFLATSTSDPSTDSTRGSPSTRRTQRPSSTTRPTTSSCRRVTIRTCGITTPTPSFRSVCK |
| | KIRLLGATSGEQSSRPRSGRKALRRLLERRDR |
| 46 | LEPRSPKPTSKSRTTAGVWCFLATSTSDPSTDSTRGSPSTRRTQRPSSTTRPTTSSCRRVTIRTCGITTPTPSFRSVCK |
| | KIRLLGATSGEQSSRPRSGFSNLSVWL |
| 47 | LEPRSPKPTSKSRTTRRTQRPSSTTRPTTSSCRRVTIRTCGITTPTPSFRSVCKKIRLLGATSGEQSSRPRSGFSNLSV |
| | WLRKALRRLLERRDR |
| 48 | LEPRSPKPTSKSRTTRRVTIRTCGITTPTPSFRSVCKKIRLLGATSGEQSSRPRSGFSNLSVWLRKALRRLLERRDR |
| 49 | LEPRSPKPTSKSRTTVCKKIRLLGATSGEQSSRPRSGFSNLSVWLRKALRRLLERRDR |

### REFERENCES

Bär S, et al. (2008) Vesicular egress of non-enveloped lytic parvoviruses depends on gelsolin functioning. PLoS Pathog. 4(8).
Bär S, et al. (2013) Vesicular Transport of Progeny Parvovirus Particles through ER and Golgi Regulates Maturation and Cytolysis. PLoS Pathog. 9(9).
Benskey M J, et al. (2016) Continuous collection of adeno-associated virus from producer cell medium significantly increases total viral yield. Hum Gene Ther Methods. 27(1):32-45.
Buller R M L, et al. (1981) Herpes Simplex Virus Types 1 and 2 Completely Help Adenovirus-Associated Virus Replication. J Virol. 40(1):241-247.
Cole C. et al. (2008) The Jpred 3 secondary structure prediction server. Nucleic Acids Res. 36:W197-201.
Colombo M. et al. (2014) Biogenesis, secretion, and intercellular interactions of exosomes and other extracellular vesicles. Annu Rev Cell Dev Biol. 30:255-289.
Escola J M. et al. (1998) Selective enrichment of tetraspan proteins on the internal vesicles of multivesicular endosomes and on exosomes secreted by human B-lymphocytes. J Biol Chem. 273(32):20121-20127.
Felsenstein J. (1985) Confidence Limits on Phylogenies: An Approach Using the Bootstrap. Evolution (NY). 39(4):783.
György B, et al. (2017) Rescue of Hearing by Gene Delivery to Inner-Ear Hair Cells Using Exosome-Associated AAV. Mol Ther. 25(2):379-391.
György B, et al. (2018) Extracellular vesicles: nature's nanoparticles for improving gene transfer with adeno-associated virus vectors. Wiley Interdiscip Rev Nanomedicine Nanobiotechnology. 10(3):e1488.
Hudry E, et al. (2016 April) Exosome-associated AAV vector as a robust and convenient neuroscience tool. Gene Ther. 23(4):380-392.
Hudry E, et al. (2016) Exosome-associated AAV vector as a robust and convenient neuroscience tool. Gene Ther. 23(4):380-392.
Janik J E, et al. (1981) Locations of adenovirus genes required for the replication of adenovirus-associated virus. Proc Natl Acad Sci USA. 78(31):1925-1929.
Keerthikumar S, et al. (2016) ExoCarta: A Web-Based Compendium of Exosomal Cargo. J Mol Biol. 428(4):688-692.
Kelley L A, et al. (2015) The Phyre2 web portal for protein modeling, prediction and analysis. Nat Protoc. 10(6): 845-858.
Kim D I, et al. (2016) An improved smaller biotin ligase for BioID proximity labeling. Mol Biol Cell. 27(8):1188-1196.
Koles K, et al. (2012) Mechanism of evenness interrupted (Evi)-exosome release at synaptic boutons. J Biol Chem. 287(20):16820-16834.
Kumar S, et al. (2016) MEGA7: Molecular Evolutionary Genetics Analysis Version 7.0 for Bigger Datasets. Mol Biol Evol. 33(7):1870-1874.
Lock M, et al. (2010) Rapid, simple, and versatile manufacturing of recombinant adeno-associated viral vectors at scale. Hum Gene Ther. 21(10):1259-71
Maguire C A, et al. Microvesicle-associated AAV vector as a novel gene delivery system. Mol Ther. 20(5):960-71.
Mathivanan S. et al. (2009) ExoCarta: A compendium of exosomal proteins and RNA. Proteomics. 9(21):4997-5000.
Meier A F, et al. (2020) The interplay between adeno-associated virus and its helper viruses. Viruses. 12(6):662.
Meliani A, et al. (2017) Enhanced liver gene transfer and evasion of preexisting humoral immunity with exosome-enveloped AAV vectors. Blood Adv. 1(23):2019-2031.
Ogden P J, et al. (2009) Comprehensive AAV capsid fitness landscape reveals a viral gene and enables machine-guided design. Science. 366(6469):1139-1143.
Okada T, et al. (2009) Scalable purification of adeno-associated virus serotype 1 (AAV1) and AAV8 vectors, using dual ion-exchange adsorptive membranes. Hum Gene Ther. 20(9):1013-1021.
Okonechnikov K, et al. (2012) Unipro UGENE: A unified bioinformatics toolkit. Bioinformatics. 28(8):1166-1167.
Orefice N S, et al. (2019) Real-Time Monitoring of Exosome Enveloped-AAV Spreading by Endomicroscopy Approach: A New Tool for Gene Delivery in the Brain. Mol Ther—Methods Clin Dev. 14:237-251.
Pegtel D M, et al. (2019) Exosomes. Annu Rev Biochem. 88:487-514.
Piras B A. et al. (2016) Distribution of AAV8 particles in cell lysates and culture media changes with time and is dependent on the recombinant vector. Mol Ther Methods Clin Dev. 3:16015.
Saitou N, et al. (1987) The neighbor-joining method: a new method for reconstructing phylogenetic trees. Mol Biol Evol. 4(4):406-25.
Sapay N, et al. (2006) Prediction of amphipathic in-plane membrane anchors in monotopic proteins using a SVM classifier. BMC Bioinformatics. 7:255.
Savina A, et al. (2002) The exosome pathway in K562 cells is regulated by Rab11. J Cell Sci. 115(12):2505-2515.
Savina A, et al. (2005) Rab11 promotes docking and fusion of multivesicular bodies in a calcium-dependent manner. Traffic. 6(2):131-143.
Schiller L T, et al. (2018) Enhanced Production of Exosome-Associated AAV by Overexpression of the Tetraspanin CD9. Mol Ther Methods Clin Dev. 9:278-287.
Shearer U, et al. (2019) Distribution and Co-localization of endosome markers in cells. Heliyon. 5(9):e02375.
Smith G A, et al. (2002) Break ins and break outs: Viral interactions with the cytoskeleton of mammalian cells. Annu Rev Cell Dev Biol. 18:135-161.
Thompson J D, et al. (1994) CLUSTAL W: Improving the sensitivity of progressive multiple sequence alignment through sequence weighting, position-specific gap penalties and weight matrix choice. Nucleic Acids Res. 22(22):4673-4680.
Uloha A I, et al. (2003) The relationship between insulin-dependent diabetes mellitus and acute infections in children. Endokrynologia, Diabetologia i Choroby Przemiany Materii Wieku Rozwojowego. 9(2):73-76.
Van Niel G, et al. (2018) Shedding light on the cell biology of extracellular vesicles. Nat Rev Mol Cell Biol. 19(4):213-228.
Vandenberghe L H, et al. (2010) Efficient serotype-dependent release of functional vector into the culture medium during adeno-associated virus manufacturing. Hum Gene Ther. 21(10):1251-1257.
Volak A. et al. (2018) Virus vector-mediated genetic modification of brain tumor stromal cells after intravenous delivery. J Neurooncol. 139(2):293-305
Wistuba A, et al. (1997) Subcellular compartmentalization of adeno-associated virus type 2 assembly. J Virol. 71(2):1341-1352.

What is claimed is:

1. A membrane-associated accessory protein (MAAP) derived from an alternate reading frame in the genome sequence of an Adeno-Associated Virus (AAV),
wherein MAAP promotes the formation of extracellular vesicles and/or AAV particles in a mammalian cell; and
wherein MAAP comprises the sequence set forth in any one of SEQ ID NO. 01-SEQ ID NO:15.

2. A membrane-associated accessory protein (MAAP) derived from an alternate reading frame in the genome sequence of an Adeno-Associated Virus (AAV),
wherein MAAP promotes the formation of extracellular vesicles and/or AAV particles in a mammalian cell; and
wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15.

3. A membrane-associated accessory protein (MAAP) derived from an alternate reading frame in the genome sequence of an Adeno-Associated Virus (AAV),
wherein MAAP associates with extracellular vesicles and/or AAV particles secreted from a mammalian cell; and
wherein MAAP comprises the sequence set forth in any one of SEQ ID NO:01-SEQ ID NO:15.

4. A membrane-associated accessory protein (MAAP) derived from an alternate reading frame in the genome sequence of an Adeno-Associated Virus (AAV),
wherein MAAP associates with extracellular vesicles and/or AAV particles secreted from a mammalian cell; and
wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in any one of SEQ ID NO:01-SEQ ID NO:15.

5. A membrane-associated accessory protein (MAAP) derived from an alternate reading frame in the genome sequence of an Adeno-Associated Virus (AAV),
wherein MAAP promotes the formation of extracellular vesicles and/or AAV particles secreted from a mammalian cell; and
wherein MAAP comprises the sequence set forth in any one of SEQ ID NO:36-SEQ ID NO:49.

6. A membrane-associated accessory protein (MAAP) derived from an alternate reading frame in the genome sequence of an Adeno-Associated Virus (AAV),
wherein MAAP associates with extracellular vesicles and/or AAV particles secreted from a mammalian cell; and
wherein MAAP comprises the sequence set forth in any one of SEQ ID NO:36-SEQ ID NO:49.

7. The membrane-associated accessory protein (MAAP) of claim 2 or 4, wherein MAAP comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in SEQ ID NO:08.

8. The membrane-associated accessory protein (MAAP) of any one of claims 1-6, wherein MAAP covalently or non-covalently attaches to one or more of a polypeptide, a glycopeptide, a polysaccharide, a glycolipid, a lipid, a nucleic acid polymer, or a combination thereof.

9. The membrane-associated accessory protein (MAAP) of any one of claims 1-6, wherein MAAP covalently or non-covalently attaches to one or more therapeutic agents.

10. The membrane-associated accessory protein (MAAP) of claim 9, wherein the one or more therapeutic agents comprise an oligonucleotide therapeutic agent.

11. The membrane-associated accessory protein (MAAP) of claim 10, wherein the oligonucleotide therapeutic agent comprises a single-stranded or double-stranded DNA, iRNA, shRNA, siRNA, mRNA, non-coding RNA (ncRNA), an antisense molecule, miRNA, a morpholino, a peptide-nucleic acid (PNA), or an analog or conjugate thereof.

12. The membrane-associated accessory protein (MAAP) of claim 10, wherein the oligonucleotide therapeutic agent comprises a CRISPR-based endonuclease.

13. The membrane-associated accessory protein (MAAP) of any one of claims 1-6, wherein MAAP is encapsulated in one or more extracellular vesicles and/or AAV particles in the cell, wherein the one more or more extracellular vesicles and/or AAV particles are secreted by the cell.

14. The membrane-associated accessory protein (MAAP) of any one of claims 1-6, wherein MAAP is encapsulated in one or more nanoparticles in the cell, wherein the one more or more nanoparticles are secreted by the cell.

15. The membrane-associated accessory protein (MAAP) of any one of claims 1-6, wherein MAAP is covalently attached or non-covalently attached to an AAV capsid.

16. A modified AAV capsid gene sequence, comprising: the sequence set forth in any one of SEQ ID NO:16-SEQ ID NO:30,
wherein the sequence comprises one or more modifications; and
wherein the one or more modifications alters a cell's ability to secrete extracellular vesicles and/or AAV particles.

17. The modified AAV capsid gene sequence of claim 16, wherein the one or more modifications can be at any position of the sequence.

18. The modified AAV capsid gene sequence of claim 16, wherein the cell's altered ability affects the amount of extracellular vesicles and/or AAV particles secreted by the cell.

19. The modified AAV capsid gene sequence of claim 16, wherein the cell's altered ability affects the rate of formation of extracellular vesicles and/or AAV particles in the cell.

20. The modified AAV capsid gene sequence of claim 16, wherein the cell is a mammalian cell.

21. The modified AAV capsid gene sequence of claim 16, wherein the cell is a eukaryotic cell.

22. The modified AAV capsid gene sequence of claim 16, wherein the cell is in culture.

23. The modified AAV capsid gene sequence of claim 16, wherein the cell is a human cell.

24. The modified AAV capsid gene sequence of claim 16, wherein the cell is in a subject.

25. The modified AAV capsid gene sequence of claim 24, wherein the subject is a human.

26. An isolated nucleic acid molecule, comprising: a nucleic acid sequence encoding a polypeptide for promoting the formation of extracellular vesicles and/or AAV particles in cell.

27. An isolated nucleic acid molecule, comprising: a nucleic acid sequence encoding a polypeptide associated with extracellular vesicles and/or AAV particles secreted from a cell.

28. The isolated nucleic acid molecule of claim 26 or 27, wherein the encoded polypeptide modulates formation of extracellular vesicles and/or AAV particles.

29. The isolated nucleic acid molecule of claim 26 or 27, wherein the encoded polypeptide modulates the rate or efficiency of extracellular vesicle and/or AAV particle secretion.

30. The isolated nucleic acid molecule of claim 28 or 29, wherein modulating comprises increasing the rate or efficiency of extracellular vesicle and/or AAV particle secretion.

31. The isolated nucleic acid molecule of claim 28 or 29, wherein modulating comprises decreasing the rate or efficiency of extracellular vesicle and/or AAV particle secretion.

32. The isolated nucleic acid molecule of claim 26 or 27, wherein the encoded polypeptide comprises a membrane-associated accessory protein (MAAP) or a fragment thereof.

33. The isolated nucleic acid molecule of claim 32, wherein MAAP comprises an N-terminal hydrophobic domain linked to cationic, amphipathic C-terminal domain.

34. The isolated nucleic acid molecule of claim 26 or 27, wherein the encoded polypeptide comprises the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO: 12, SEQ ID NO:13, SEQ ID NO:14, SEQ ID NO:15, or a fragment thereof.

35. The isolated nucleic acid molecule of claim 26 or 27, wherein the encoded polypeptide comprises a sequence having at least 30%, at least 40%, at least 50%, at least 60%, at least 70%, at least 80%, or at least 90% identity to the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof.

36. The isolated nucleic acid molecule of claim 26 or 27, wherein the encoded polypeptide comprises a sequence having at least 30%, at least 35%, at least 40%, at least 45%, at least 50%, at least 55%, at least 60% identity, at least 65%, at least 70%, at least 75%, at least 80%, at least 85%, at least 90%, or 95% identity to the sequence set forth in SEQ ID NO:08 or a fragment thereof.

37. The isolated nucleic acid molecule of claim 26 or 27, wherein the encoded polypeptide comprises the sequence set forth in any one of SEQ ID NO:36-SEQ ID NO:49.

38. The isolated nucleic acid molecule of claim 26 or 27, wherein the nucleic acid sequence comprises the sequence set forth in SEQ ID NO: 16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

39. A vector, comprising: the isolated nucleic acid molecule of claim 26 or 27.

40. The vector of claim 39, wherein the vector is an AAV vector.

41. The vector of claim 39, wherein the vector comprises one or more regulatory elements.

42. The vector of claim 41, wherein the vector comprises a promoter operably linked to the isolated nucleic acid molecule, wherein the promoter drives the expression of the encoded polypeptide.

43. The vector of claim 42, wherein the promoter comprises a constitutive promoter, a cell-specific promoter, or a regulatable promoter element.

44. A method of enhancing secretion of extracellular vesicles and/or AAV particles from a cell, comprising:
delivering to a cell an isolated nucleic acid molecule comprising a nucleic acid sequence encoding a polypeptide for promoting the formation of extracellular vesicles and/or AAV particles in a cell or a polypeptide associated with extracellular vesicles and/or AAV particles secreted from a cell; and
expressing the encoded polypeptide.

45. The method of claim 44, wherein the nucleic acid sequence encodes a fusion product, wherein the fusion product comprises at least the encoded polypeptide.

46. The method of claim 44, further comprising encapsulating the encoded polypeptide in extracellular vesicles and/or AAV particles.

47. The method of claim 44, further comprising secreting extracellular vesicles and/or AAV particles from the cell.

48. The method of claim 44, further comprising encapsulating one or more of a polypeptide, a glycopeptide, a polysaccharide, a glycolipid, a lipid, a nucleic acid polymer, or to a combination thereof in extracellular vesicles and/or AAV particles.

49. The method of claim 44, further comprising encapsulating one or more therapeutic agents in extracellular vesicles and/or AAV particles.

50. The method of claim 44, wherein the encoded polypeptide increases the rate or efficiency of extracellular vesicle and/or AAV particle secretion.

51. The method of claim 44, wherein the encoded polypeptide comprises a membrane-associated accessory protein (MAAP) or a fragment thereof.

52. The method of claim 44, wherein the encoded polypeptide comprises the sequence set forth in SEQ ID NO:01, SEQ ID NO:02, SEQ ID NO:03, SEQ ID NO:04, SEQ ID NO:05, SEQ ID NO:06, SEQ ID NO:07, SEQ ID NO:08, SEQ ID NO:09, SEQ ID NO:10, SEQ ID NO:11, SEQ ID NO:12, SEQ ID NO:13, SEQ ID NO:14, SEQ ID NO:15, or a fragment thereof.

53. The method of claim 44, wherein the encoded polypeptide comprises the sequence set forth in SEQ ID NO:36, SEQ ID NO:37, SEQ ID NO:38, SEQ ID NO:39, SEQ ID NO:40, SEQ ID NO:41, SEQ ID NO:42, SEQ ID NO:43, SEQ ID NO:44, SEQ ID NO:45, SEQ ID NO:46, SEQ ID NO:47, SEQ ID NO:48, or SEQ ID NO:49.

54. The method of claim 44, wherein the encoded polypeptide comprises a sequence having at least 30%, at least 35%, at least 40%, at least 45%, at least 50%, at least 55%, at least 60%, at least 65%, at least 70%, at least 75%, at least 80%, at least 85%, at least 90%, or at least 95% identity to the sequence set forth in SEQ ID NO:08 or a fragment thereof.

55. The method of claim 44, wherein the encoded polypeptide comprises a sequence set forth in any one of SEQ ID NO:36-SEQ ID NO:49.

56. The method of claim 44, wherein the nucleic acid sequence for the polypeptide comprises the sequence set forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

57. The method of claim 44, wherein the nucleic acid sequence encoding the polypeptide comprises a sequence having at least 30%, at least 40%, at least 50%, at least 60%, at least 70%, at least 80%, or at least 90% identity forth in SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, or a fragment thereof.

58. The method of claim 44, wherein delivering the isolated nucleic acid molecule comprises using a vector.

59. The method of claim 58, wherein the vector is an AAV vector.

60. The method of claim 44, wherein the cell is a mammalian cell.

61. The method of claim 44, wherein the cell is in culture.

62. The method of claim 61, comprising harvesting the secreted extracellular vesicles and/or AAV particles from conditioned media of the culture.
